SEQUENCE LISTING (5 sequences)

SEQ ID NO: 1 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 2 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIDTLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 3 - DNA, 12 bp, synthetic construct
  source 1..12: mol_type = other DNA
atgcatgcatgc

SEQ ID NO: 4 - DNA, 12 bp, synthetic construct
  source 1..12: mol_type = other DNA
gcatgcatgcat

SEQ ID NO: 5 - DNA, 18 bp, synthetic construct
  source 1..18: mol_type = other DNA
atgcataaaaaagcatgc

(19) United States
(12) Patent Application Publication
(10) Pub. No.: US 2025/0059586 A1
(43) Pub. Date: Feb. 20, 2025

(54) NON-OPAQUE LYTIC BUFFER COMPOSITION FORMULATIONS

(71) Applicant: Becton, Dickinson and Company, Franklin Lakes, NJ (US)

(72) Inventors: James Petisce, Westford, MA (US); Chang Chen, Cary, NC (US); Meghan Elizabeth Wolfgang, Pittsboro, NC (US); Honghua Zhang, San Diego, CA (US); Tanya Ferguson, San Diego, CA (US); James Nealis, Apex, NC (US)

(21) Appl. No.: 18/721,639

(22) PCT Filed: Feb. 3, 2023
(86) PCT No.: PCT/US2023/061980

§ 371 Date: Jun. 18, 2024

Related U.S. Application Data
(60) Provisional application No. 63/307,092, filed on Feb. 5, 2022.

(51) Int. Cl. C12Q 1/6806; C12Q 1/6844

(52) U.S. Cl. CPC C12Q 1/6806; C12Q 1/6844

(57) ABSTRACT

Disclosed herein include methods, compositions, and kits for use in detecting a target nucleic acid sequence in a sample. Provided include storage-stable lysis buffers. In some embodiments, the lysis buffer comprises: one or more surfactants and magnesium sulfate (MgSO₄). In some embodiments, the formation of a precipitate (for example, a complex of Mg⁺² and the one or more surfactants) is substantially inhibited for a period of time under at least one storage condition.

### RELATED APPLICATIONS

This present application is a National Stage application of PCT Application PCT/US2023/061980, filed on Feb. 3, 2023, which claims the benefit under 35 U.S.C. § 119 (e) of U.S. Provisional Patent Application Ser. No. 63/307,092, filed Feb. 5, 2022, the content of this related application is incorporated herein by reference in its entirety for all purposes.

### REFERENCE TO SEQUENCE LISTING

The present application is being filed along with a Sequence Listing in electronic format. The Sequence Listing is provided as a file entitled 68EB-317336-WO, created Feb. 2, 2023, which is 8.0 kilobytes in size. The information in the electronic format of the Sequence Listing is incorporated herein by reference in its entirety.

### BACKGROUND

### Field

The present disclosure relates generally to methods and compositions for amplification (e.g., isothermal amplification) of nucleic acids.

### Description of the Related Art

Nucleic acid-based diagnostics can be useful for rapid detection of infection, disease and/or genetic variations. For example, identification of bacterial or viral nucleic acid in a sample can be useful for diagnosing a particular type of infection. Other examples include identification of single nucleotide polymorphisms for disease management or forensics, and identification of genetic variations indicative of genetically modified food products. Often, nucleic acid-based diagnostic assays require amplification of a specific portion of nucleic acid in a sample. A common technique for nucleic acid amplification is the polymerase chain reaction (PCR). This technique typically requires a cycling of temperatures (i.e., thermocycling) to proceed through the steps of denaturation (e.g., separation of the strands in the double-stranded DNA (dsDNA) complex), annealing of oligonucleotide primers (short strands of complementary DNA sequences), and extension of the primer along a complementary target by a polymerase. Such thermocycling can be a time consuming process that generally requires specialized machinery. Thus, a need exists for quicker nucleic acid amplification methods that can be performed without thermocycling. Such methods may be useful, for example, for on-site testing and point-of-care diagnostics. Moreover, there is a need for such compositions and methods of nucleic acid detection wherein lytic buffers employed to lyse biological entities (e.g., viral particles, bacteria) are stable during storage (e.g., do not become opaque due to precipitates). There is a need for lysis buffers that (i) lyse a sufficient percent of organisms to maintain clinical performance; (ii) are stable for 18 months when stored refrigerated or at room temperature; (iii) do not destroy the nucleic acid within the test sample during lysis; and (iv) are effective in the presence of clinical matrix.

### SUMMARY

Disclosed herein include lysis buffers. In some embodiments, the lysis buffer comprises: one or more surfactants; ammonium sulfate ((NH₄)₂SO₄); and magnesium sulfate (MgSO₄), wherein the lysis buffer does not comprise one or more of sodium dodecyl sulfate (SDS) and cetyl trimethylammonium bromide (CTAB), and wherein formation of a precipitate is substantially inhibited for a period of time under a storage condition. In some embodiments, the appearance of the precipitate in the lysis buffer does not occur for at least about twenty days during the storage condition.

The lysis buffer can comprise: one or more surfactants; (NH₄)₂SO₄ and MgSO₄, wherein formation of a precipitate is substantially inhibited for a period of time under a storage condition, and wherein the appearance of the precipitate in the lysis buffer does not occur, or the precipitate formed in the lysis buffer is not detectable, for at least about twenty days during the storage condition. The lysis buffer can comprise one or more chelators (e.g., EDTA, EGTA). For example, lysis buffers can comprise EDTA, EGTA, or the like as a metal ion chelator, which can, in some embodiments, form stronger complexes with heavy metal ions or calcium than magnesium ion. In some embodiments, the chelator is present at a concentration of about 0.1 mM, 0.2 mM, 0.3 mM, 0.4 mM, 0.5 mM, 0.6 mM, 0.7 mM, 0.8 mM, 0.9 mM, 1 mM, or a range or number between any of these values.

In some embodiments, the lysis buffer does not comprise SDS, CTAB, or both. In some embodiments, the precipitate (the formation of which is substantially inhibited for a period of time under a storage condition in the methods and compositions disclosed herein) is or comprises a complex of Mg⁺² and the one or more surfactants. In some embodiments, the lysis buffer is a precipitation-stable aqueous composition (e.g., an aqueous composition wherein precipitation is absent or suppressed for an extended period of time under a storage condition). In some embodiments, the lysis buffer is a precipitation-free aqueous composition. In some embodiments, the precipitation of complexes consisting of Mg⁺² and the surfactant is not present. In some embodiments, the precipitation of complexes consisting of Mg⁺² and the surfactant is eliminated. In some embodiments, the precipitation of complexes consisting of Mg⁺² and the surfactant is suppressed. In some embodiments, the concentration of soluble Mg⁺² is not reduced more than about 1.1-fold relative to the start of the period of time. In some embodiments, the appearance of the precipitate in the lysis buffer does not occur for at least about thirty days, about sixty days, about ninety days, about six months, about a year, or about two years, during the storage condition.

The storage condition can, for example, comprise transport of the lysis buffer. In some embodiments, the storage condition comprises thermal stress, one or more freeze-thaw cycles, sonication, shear forces, agitation, pressure changes, light irradiation, or any combination thereof. In some embodiments, the storage condition comprises ambient conditions (e.g., in the range from about 20° C. to about 25° C.). In some embodiments, the storage condition comprises refrigeration conditions (e.g., about 4° C.). In some embodiments, the storage condition comprises 14° C. In some embodiments, the period of time is at least about thirty days, about sixty days, about ninety days, about six months, about a year, or about two years.

In some embodiments, the substantial inhibition of formation of the precipitate comprises the lysis buffer having no visible particulates as assessed by visual inspection. In some embodiments, the absence of the precipitate in the lysis buffer comprises the lysis buffer as assessed by visual inspection. In some embodiments, the appearance of the precipitate in the lysis buffer comprises the lysis buffer having visible particulates as assessed by visual inspection. In some embodiments, precipitation is monitored by using light scattering. In some embodiments, precipitation is monitored using a turbidity sensor, turbidimeter, or nephelometer. In some embodiments, precipitation is monitored using a spectrophotometer. In some embodiments, the substantial inhibition of formation of the precipitate comprises the lysis buffer having an absorbance at a wavelength below a threshold absorbance as determined by spectrophotometric analysis. In some embodiments, the appearance of the precipitate in the lysis buffer comprises the lysis buffer having an absorbance at a wavelength above a threshold absorbance as determined by spectrophotometric analysis. In some embodiments, the threshold absorbance is about 0.001 to about 6.0 absorbance units (AU). In some embodiments, the appearance of the precipitate in the lysis buffer comprises absorbance at a wavelength greater than about 1.1-fold the absorbance at said wavelength at the start of the period of time as determined by spectrophotometric analysis. In some embodiments, the substantial inhibition of formation of the precipitate comprises the lysis buffer having an absorbance at a wavelength less than about 1.1-fold the absorbance at said wavelength at the start of the period of time as determined by spectrophotometric analysis. In some embodiments, the spectrophotometric analysis is conducted with a spectrophotometer at a wavelength of about 200 nm to about 900 nm. In some embodiments, the wavelength is the wavelength that maximizes absorbance of the precipitate. In some embodiments, the absorbance value is due to light scattering of the precipitate. The spectrophotometer can be a UV-Vis spectrophotometer, an IR spectrophotometer, a Visible-Near IR spectrophotometer, a Raman spectrophotometer, or a combination thereof.

In some embodiments, the lysis buffer further comprises one or more alcohols. In some embodiments, the one or more alcohols have a carbon chain length of 1, 2, 3, 4, 5, or 6. In some embodiments, the one or more alcohols are selected from the group comprising ethanol, isopropanol, isobutyl alcohol, pentanol, and hexanol. In some embodiments, the one or more alcohols comprises about 0.001% (v/v) to about 20.0% (v/v) of the lysis buffer. In some embodiments, the lysis buffer comprises the one or more alcohols at about 0.001% (v/v) to about 20.0% (v/v), for example about 0.1% (v/v) to about 4.0% (v/v).

In some embodiments, the MgSO₄ is present at a concentration of about 0.1 mM to about 100 mM, for example about 4 mM. In some embodiments, the (NH₄)₂SO₄ is present at a concentration of about 0.1 mM to about 100 mM, for example about 5 mM. In some embodiments, the (NH₄)₂SO₄ is present at a concentration of about 10 mM, and wherein the appearance of the precipitate in the lysis buffer is delayed by at least about ten days (under a storage condition described herein) as compared a comparable lysis buffer wherein the (NH₄)₂SO₄ is present at a concentration of about 5 mM.

The lysis buffer can comprise: an acid. In some embodiments, the acid comprises an organic acid, an inorganic acid, or a mixture thereof. In some embodiments, the inorganic acid is hydrogen chloride (HCl). In some embodiments, the acid is present at a concentration of about 8.8 mM. The lysis buffer can comprise: a pH buffer. In some embodiments, the pH buffer comprises glycine and HCl. In some embodiments, the pH buffer comprises 10.0 mM glycine. In some embodiments, the pH of the lysis buffer is about 1.0 to about 6.0, for example about 2.2.

In some embodiments, the one or more surfactants are capable of lysing biological entities to release sample nucleic acids comprised therein. In some embodiments, the sample nucleic acids comprise sample ribonucleic acids and/or sample deoxyribonucleic acids. In some embodiments, the biological entities comprise a virus, a bacteria, a fungi, a protozoa, portions thereof, or any combination thereof. In some embodiments, the biological entities comprise one or more of prokaryotic cells, eukaryotic cells, viral particles, exosomes, protoplasts, and microvesicles. In some embodiments, the one or more surfactants comprise about 0.001% (w/v) to about 10.0% (w/v) of the lysis buffer. In some embodiments, the one or more surfactants comprise one or more of a cationic surfactant, an anionic surfactant, a non-ionic surfactant, and an amphoteric surfactant. In some embodiments, the lysis buffer further comprises a tween surfactant. In some embodiments, the tween surfactant is selected from the group consisting of Tween 20, Tween 40, Tween 45, Tween 60, Tween 65, Tween 80, Tween 81 and Tween 85. In some embodiments, the tween surfactant comprises about 0.01% to 0.2% (w/v) of the lysis buffer.

In some embodiments, the one or more surfactants comprises CTAB. In some embodiments, the lysis buffer comprises about 0.2% (w/v) CTAB and the (NH₄)₂SO₄ is present at a concentration of about 5 mM. In some embodiments, the one or more surfactants comprises CTAC. In some embodiments, the lysis buffer comprises about 0.2% (w/v) CTAC and the (NH₄)₂SO₄ is present at a concentration of about 5 mM. In some embodiments, the one or more surfactants comprises SDS. In some embodiments, the lysis buffer comprises about 0.4% (w/v) SDS and the (NH₄)₂SO₄ is present at a concentration of about 10 mM. In some embodiments, the lysis buffer further comprises Tween 80. In some embodiments, the one or more surfactants comprises sodium decylsulfate (SDeS). In some embodiments, the lysis buffer comprises about 0.2% (w/v) SDeS and the (NH₄)₂SO₄ is present at a concentration of about 5 mM. In some embodiments, the one or more surfactants comprises SDeS. In some embodiments, the lysis buffer comprises about 0.2% (w/v) SDeS and the (NH₄)₂SO₄ is present at a concentration of about 10 mM. In some embodiments, the one or more surfactants comprises SDeS. In some embodiments, the lysis buffer comprises about 0.4% (w/v) SDeS and the (NH₄)₂SO₄ is present at a concentration of about 10 mM. In some embodiments, the one or more surfactants comprises SDeS. In some embodiments, the lysis buffer comprises about 0.8% (w/v) SDeS and the (NH₄)₂SO₄ is present at a concentration of about 10 mM. In some embodiments, the one or more surfactants comprises sodium decyl sulfate (SDeS). In some embodiments, the lysis buffer comprises about 0.4% (w/v) SDeS and the (NH₄)₂SO₄ is present at a concentration of about 10 mM. In some embodiments, the lysis buffer further comprises Tween 80. In some embodiments, the one or more surfactants comprises CTAB. In some embodiments, the lysis buffer comprises about 0.2% (w/v) CTAB and the (NH₄)₂SO₄ is present at a concentration of about 5 mM. In some embodiments, the one or more surfactants comprises CTAC. In some embodiments, the lysis buffer comprises about 0.2% (w/v) CTAC and the (NH₄)₂SO₄ is present at a concentration of about 5 mM. In some embodiments, the one or more surfactants comprises sodium octyl sulfate (S Octyl S). In some embodiments, the lysis buffer comprises about 0.2% (w/v) S Octyl S and the (NH₄)₂SO₄ is present at a concentration of about 5 mM.

Disclosed herein include methods of processing a sample. In some embodiments, the method comprises: (a) contacting a sample comprising biological entities with a lysis buffer provided herein to generate a treated sample, wherein the lysis buffer is capable of lysing biological entities to release sample nucleic acids comprised therein.

In some embodiments, the sample nucleic acids are suspected of comprising a target nucleic acid sequence, the method further comprising detecting the target nucleic acid sequence in the sample. In some embodiments, detecting the target nucleic acid sequence in the sample comprises: (b) contacting a reagent composition (e.g., a wet composition, a dried composition) with the treated sample to generate an amplification reaction mixture, wherein the reagent composition comprises one or more amplification reagents; (c) amplifying a target nucleic acid sequence in the amplification reaction mixture, thereby generating a nucleic acid amplification product; and (d) detecting the nucleic acid amplification product, wherein the detecting is performed in less than about 20 minutes from the time the reagent composition is contacted with the treated sample.

In some embodiments, the sample nucleic acids comprise sample ribonucleic acids and/or sample deoxyribonucleic acids. In some embodiments, the sample ribonucleic acids comprise a cellular RNA, a mRNA, a microRNA, a bacterial RNA, a viral RNA, or any combination thereof. In some embodiments, the one or more amplification reagents comprise a reverse transcriptase and/or an enzyme having a hyperthermophile polymerase activity. In some embodiments, the enzyme having a hyperthermophile polymerase activity has a reverse transcriptase activity. In some embodiments, contacting the reagent composition with the treated sample comprises dissolving the reagent composition in the treated sample. In some embodiments, the reagent composition comprises one or more of a reverse transcriptase, an enzyme having a hyperthermophile polymerase activity, a first primer, a second primer, and a reverse transcription primer. In some embodiments, the amplifying is performed in an isothermal amplification condition. In some embodiments, detecting the nucleic acid amplification product comprises use of a real-time detection method.

In some embodiments, the reagent composition is lyophilized and/or heat-dried (e.g., a dried composition) and comprises one or more additives. In some embodiments, the one or more additives comprise: an amino acid; a sugar or sugar alcohol (e.g., lactose, trehalose, dextran, erythritol, arabitol, xylitol, sorbitol, mannitol, or any combination thereof); and/or a polymer (e.g., polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof).

In some embodiments, the sample nucleic acids comprise a nucleic acid comprising the target nucleic acid sequence. In some embodiments, the target nucleic acid sequence comprises a first strand and a second strand complementary to each other. In some embodiments, amplifying the target nucleic acid sequence comprises: amplifying a target nucleic acid sequence comprising a first strand and a second strand complementary to each other in an isothermal amplification condition, wherein the amplifying comprises contacting a nucleic acid comprising the target nucleic acid sequence with: i) a first primer and a second primer, wherein the first primer is capable of hybridizing to a sequence of the first strand of the target nucleic acid sequence, and the second primer is capable of hybridizing to a sequence of the second strand of the target nucleic acid sequence; and ii) an enzyme having a hyperthermophile polymerase activity, thereby generating a nucleic acid amplification product, wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long. In some embodiments, the amplifying does not comprise using any enzyme other than the enzyme having a hyperthermophile polymerase activity. In some embodiments, the amplifying does not comprise heat denaturing and/or enzymatic denaturing the nucleic acid. In some embodiments, the method does not comprise contacting the nucleic acid with a single-stranded DNA binding protein prior to or during step (c).

In some embodiments, the nucleic acid is a double-stranded DNA. In some embodiments, the nucleic acid is a product of reverse transcription reaction. In some embodiments, the nucleic acid is a product of reverse transcription reaction generated from sample ribonucleic acids. In some embodiments, step (c) comprises generating the nucleic acid by a reverse transcription reaction. In some embodiments, the sample nucleic acids comprise sample ribonucleic acids, and wherein the method comprises contacting sample ribonucleic acids with a reverse transcriptase and/or a reverse transcription primer to generate a cDNA. In some embodiments, amplifying the target nucleic acid sequence comprises: (c1) contacting sample ribonucleic acids with a reverse transcriptase and/or a reverse transcription primer to generate a cDNA; (c2) contacting the cDNA with an enzyme having a hyperthermophile polymerase activity to generate a double-stranded DNA (dsDNA), wherein the dsDNA comprises a target nucleic acid sequence, and wherein the target nucleic acid sequence comprises a first strand and a second strand complementary to each other; (c3) amplifying the target nucleic acid sequence under an isothermal amplification condition, wherein the amplifying comprises contacting the dsDNA with: (i) a first primer and a second primer, wherein the first primer is capable of hybridizing to a sequence of the first strand of the target nucleic acid sequence, and the second primer is capable of hybridizing to a sequence of the second strand of the target nucleic acid sequence; and (ii) the enzyme having a hyperthermophile polymerase activity, thereby generating a nucleic acid amplification product, wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

In some embodiments, the method does not comprise using any enzymes other than the reverse transcriptase and the enzyme having a hyperthermophile polymerase activity. In some embodiments, step (d) further comprises determining the amount of the dsDNA and/or nucleic acid that comprises the target nucleic acid sequence in the sample. In some embodiments, the enzyme having a hyperthermophile polymerase activity has an amino acid sequence that is at least about 90% or at least about 95% identical to the amino acid sequence of SEQ ID NO: 1 or a functional fragment thereof. In some embodiments, the enzyme having a hyperthermophile polymerase activity is a polymerase comprising the amino acid sequence of SEQ ID NO: 1. In some embodiments, the enzyme having a hyperthermophile polymerase activity has low or no exonuclease activity. In some embodiments, amplifying the target nucleic acid sequence is performed at a constant temperature of about 55° C. to about 75° C., for example about 65° C.

In some embodiments, the first primer, the second primer, and/or the reverse transcription primer is about 8 to 16 bases long. In some embodiments, the first primer, the second primer, and/or the reverse transcription primer comprises one or more of DNA bases, modified DNA bases, or a combination thereof. In some embodiments, the nucleic acid amplification product is about 20 to 40 bases long. In some embodiments, the spacer sequence comprises a portion of the target nucleic acid sequence. In some embodiments, the spacer sequence is 1 to 10 bases long. The method can comprise: contacting the nucleic acid amplification product with a signal-generating oligonucleotide capable of hybridizing to the amplification product, wherein the single-generating oligonucleotide comprises a fluorophore, a quencher, or both. In some embodiments, detecting the nucleic acid amplification product comprises detecting a fluorescent signal. In some embodiments, the fluorescent signal is from a molecular beacon. In some embodiments, the method is performed in a single reaction vessel.

In some embodiments, the biological entities comprise one or more of prokaryotic cells, eukaryotic cells, viral particles, exosomes, protoplasts, and microvesicles. In some embodiments, the biological entities comprise a virus, a bacteria, a fungi, a protozoa, portions thereof, or any combination thereof. In some embodiments, the target nucleic acid sequence is a nucleic acid sequence of a virus, bacteria, fungi, or protozoa. In some embodiments, the sample nucleic acids are derived from a virus, bacteria, fungi, or protozoa. In some embodiments, the virus comprises one or more of SARS-COV-2, Human Immunodeficiency Virus Type 1 (HIV-1), Human T-Cell Lymphotrophic Virus Type 1 (HTLV-1), Hepatitis B Virus (HBV), Hepatitis C Virus (HCV), Herpes Simplex, Herpesvirus 6, Herpesvirus 7, Epstein-Barr Virus, Respiratory Syncytial Virus (RSV), Cytomegalo-virus, Varicella-Zoster Virus, JC Virus, Parvovirus B19, Influenza A, Influenza B, Influenza C, Rotavirus, Human Adenovirus, Rubella Virus, Human Enteroviruses, Genital Human Papillomavirus (HPV), and Hantavirus. In some embodiments, the bacteria comprises one or more of Mycobacteria tuberculosis, Rickettsia rickettsii, Ehrlichia chaffeensis, Borrelia burgdorferi, Yersinia pestis, Treponema pallidum, Chlamydia trachomatis, Chlamydia pneumoniae, Mycoplasma pneumoniae, Mycoplasma sp., Legionella pneumophila, Legionella dumoffii, Mycoplasma fermentans, Ehrlichia sp., Haemophilus influenzae, Neisseria meningitidis, Neisseria gonorrhoeae, Streptococcus pneumonia, S. agalactiae, and Listeria monocytogenes. In some embodiments, the fungi comprises one or more of Cryptococcus neoformans, Pneumocystis carinii, Histoplasma capsulatum, Blastomyces dermatitidis, Coccidioides immitis, and Trichophyton rubrum. In some embodiments, the protozoa is Trypanosoma cruzi, Leishmania sp., Plasmodium, Entamoeba histolytica, Babesia microti, Giardia lamblia, Cyclospora sp., or Eimeria sp.

In some embodiments, the amplifying step comprises multiplex amplification of two or more target nucleic acid sequences, and wherein the detecting step comprises multiplex detection of two or more nucleic acid amplification products derived from said two or more target nucleic acid sequences. In some embodiments, the two or more target nucleic acid sequences are specific to two or more different organisms. In some embodiments, the two or more different organisms comprise one or more of SARS-COV-2, Influenza A, Influenza B, and/or Influenza C.

The amplifying can comprise, or does not comprise, one or more of the following: Archaeal Polymerase Amplification (APA), loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA). In some embodiments, the amplifying does not comprise LAMP.

In some embodiments, the method does not comprise one or more of the following: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of ribonuclease inhibitors to the treated sample; (vii) the addition of ribonuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heating denaturing and/or enzymatic denaturing of the sample nucleic acids prior to and/or during amplification; and (xiii) the addition of ribonuclease H to the treated sample or amplification reaction mixture.

### BRIEF DESCRIPTION OF THE DRAWINGS

FIGS. 1A-1B show a non-limiting exemplary schematic of an isothermal amplification reaction provided herein.

FIG. 2 depicts data related to a BioAssay colorimetric Magnesium assay on fresh DNA assay lysis solution (DALB) at time of production and precipitated DNA assay lysis solution (without precipitate visually present) 7 days later.

FIGS. 3A-3C depict data related to the effect of CTAB substitution in lysis buffer on Group A Strep assay performance. Assays with standard lysis buffer (FIG. 3A) or with lysis solutions with 0.2% CTAB (FIGS. 3B-3C) are shown. Fluorescence versus time (min) is depicted for assays with 50 cp/reaction.

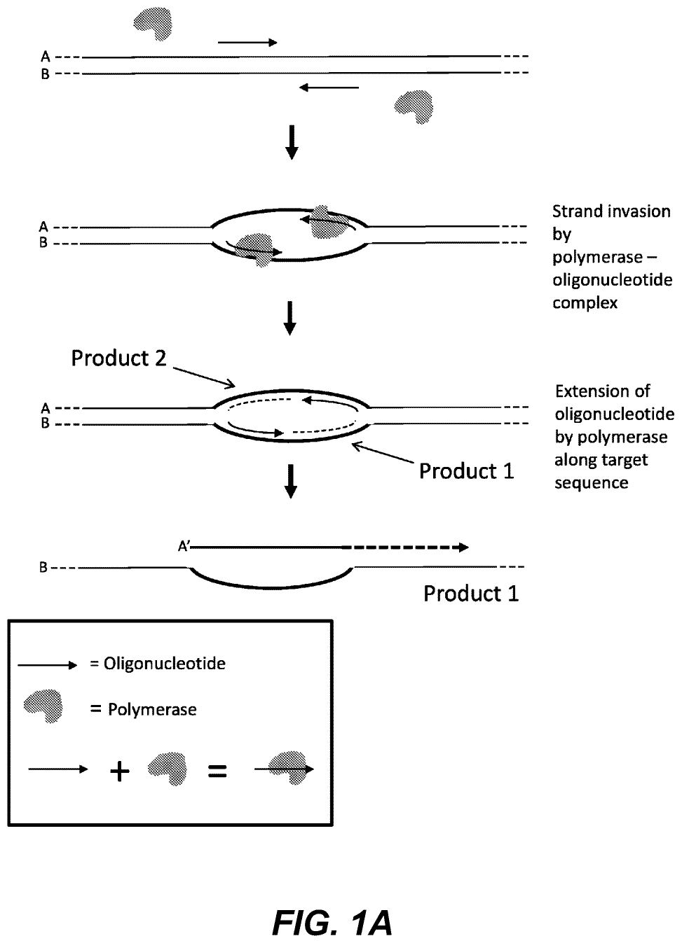

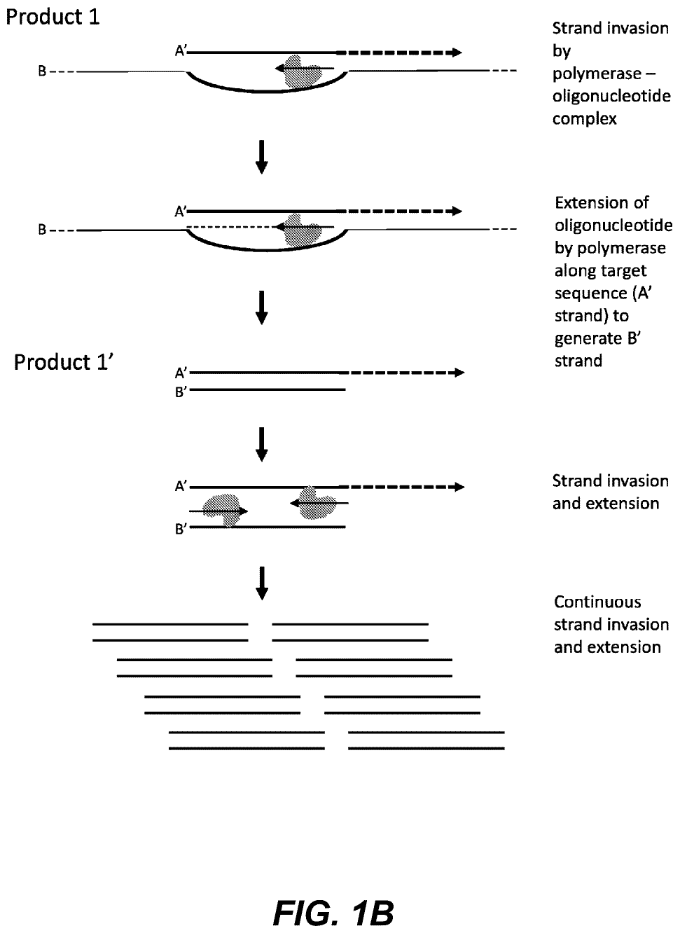

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include lysis buffers, including the lysis buffer comprising: one or more surfactants; (NH₄)₂SO₄; and MgSO₄, wherein the lysis buffer does not comprise one or more of sodium dodecyl sulfate (SDS) and cetyl trimethylammonium bromide (CTAB), and wherein formation of a precipitate is substantially inhibited for a period of time under a storage condition. In some embodiments, the appearance of the precipitate in the lysis buffer does not occur for at least about twenty days during the storage condition.

Disclosed herein include lysis buffers. In some embodiments, the lysis buffer comprises: one or more surfactants; (NH₄)₂SO₄; and MgSO₄, wherein formation of a precipitate is substantially inhibited for a period of time under a storage condition, and wherein the appearance of the precipitate in the lysis buffer does not occur for at least about twenty days during the storage condition.

Disclosed herein include methods of processing a sample. In some embodiments, the method comprises: contacting a sample comprising biological entities with a lysis buffer provided herein to generate a treated sample, wherein the lysis buffer is capable of lysing biological entities to release sample nucleic acids comprised therein.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “precipitate” shall be given its ordinary meaning, and shall also be used to refer to formation of a solid or insoluble particles in a solution. Various forms of precipitate occur and exemplary precipitates are described herein. As used herein, the term “precipitation” shall be given its ordinary meaning, and shall also be used to refer to the formation of a solid (e.g., a precipitate) in a solution. As used herein, the term “solution” shall be given its ordinary meaning, and shall also be used to refer to a substantially homogeneous mixture comprising two or more substances dissolved in a solvent. As used herein, the terms “substantially inhibit precipitation” and “inhibits precipitation” shall be given their ordinary meaning, and shall also be used to describe the inhibition of most or all visible precipitation so as to maintain homogeneity for a period of time ranging from at least 1 month to at least 2 years. As defined herein, the terms “precipitation”, “precipitate” “particulate formation”, “clouding” and “aggregation” may be used interchangeably and can refer to any physical interaction or chemical reaction which results in the “aggregation” of a complex of Mg⁺² and a surfactant.

Provided herein are methods and compositions for amplifying nucleic acid. Traditional nucleic acid amplification methods typically require a thermocycling process, nucleic acid denaturation, proteins (e.g., enzymes) that promote strand unwinding, strand separation and/or strand exchange (e.g., helicases, recombinases), and/or endonuclease agents (e.g., restriction enzymes, nicking enzymes), and often require a minimum reaction time of 20 to 30 minutes. The nucleic acid amplification methods provided herein can be performed without thermocycling, without thermal denaturation and/or enzymatic denaturation of sample nucleic acids, without added proteins (e.g., enzymes) to promote strand unwinding, strand separation and/or strand exchange, without endonuclease agents, and within a reaction time of about 10-15 minutes.

### Non-Opaque Lytic Buffer Formulations

Provided herein include methods and compositions for direct pathogen lysis in clinical samples to enable isothermal amplification using an archaeal polymerase and real-time detection of nucleic acids. Rapid point of care (POC) diagnostics can be developed that do not require sample purification. Viral particles, bacterial cells, or other pathogens still need be lysed so that their DNA and RNA can be released from the cell and can be available for an amplification reaction. Conventional chemical lysis methods (e.g., employing strong bases, ionic detergents, and chaotropic agents) are incompatible with enzyme function, as these agents will also inactivate any DNA polymerase or other enzymes. Therefore, there is significant value in an effective chemical lysis method that is compatible with enzyme function and does not require any nucleic acid purification steps to remove the lysis reagents.

Methods and compositions for rapid DNA/RNA amplification are provided herein, including Archaeal Polymerase Amplification (APA). The workflow can comprise isothermal amplification at ˜68° C., and can comprise no thermocycling. The disclosed methods and compositions can enable quick sample preparation, and can comprise chemical lysis without a purification step. The disclosed methods and compositions can include a unitized reagent-loaded disposable. Compositions and methods provided herein can employ two-color fluorescence detection based on, for example, molecular beacon or dsDNA fluor dye. The methods and compositions provided herein can be employed, for example, on the NATDx platform. The workflow can provide a sample-to-result/time-to-result (TTR) of ten minutes or less, and can comprise: (i) preheating a tube in a reader (3 min); (ii) uncapping and adding sample to 1 mL lysis buffer (1 min); (iii) closing the tube with second cap, which breaks seal and meters about 100 μL lysate into pre-heated lyophilized reagents, with a magnetic motor employed for mixing (1 min); and (iv) real-time detection at about 68° C. (5 min), with the goal of calling negative in 5 min and positive in <5 min. Lysis can take about 2 minutes at about 75-80° C. for DNA assays and about 65-70° C. for RNA assays in some embodiments.

The rapid RNA/DNA assays disclosed herein can utilize a buffer solution that chemically lyses a clinical sample or a biological sample (e.g., a harvested human sample) to enable sample analysis in no more than ten minutes. The RNA assay and the DNA assay can each have a unique lytic buffer formulation to be effective with these different test samples. There are four fundamental performance qualities for each lytic buffer solution. Lysis buffers provided herein have one or more of the following attributes (i) the solution lyses a sufficient percent of organisms to maintain clinical performance; (ii) the solution is stable for 18 months when stored refrigerated or at room temperature (with room temperature storage preferred in some embodiments for ease of user use); (iii) the solution does not destroy the nucleic acid within the test sample during lysis; and (iv) the solution is effective in the presence of clinical matrix. In some embodiments, all four properties are required for the methods and compositions provided herein. With regards to (ii), in some embodiments, room temperature storage is required, while in some embodiments, fridge storage stability is not required.

As described in Example 1, a DNA lytic buffer formulation can become opaque (with precipitates) within only two to three days after preparation when stored at either room temperature or 14° C. This performance is unacceptable because it does not meet the second fundamental performance quality described above (i.e., the solution is stable for 18 months when stored refrigerated or at room temperature). Additionally, a lytic buffer solution with an opaque appearance could be perceived by an end-user as being either damaged, non-homogeneous or unstable. There is a need for methods and compositions that eliminate the formation of the observed opacity (precipitate). There are provided, in some embodiments, approaches and compositions that mitigate formation of precipitate in lysis buffers. Disclosed herein, in some embodiments, are chemical compositions that provide a non-opaque lytic buffer formulation. Without being bound by any particular theory, numerous technical principles are related to the non-opaque formulations described herein, including salt effect, Le Chatelier's Principle, solvent polarity, common ion effect, hydrophilic-lypophilic balance and colligative properties, for example. There are provided, in some embodiments, instructions for use of the lytic buffers provided herein, including include the composition of the lytic buffer. In some embodiments provided herein, lysis buffers demonstrate stability during accelerating aging approaches. As described herein, a number of solution pathways were initially considered and evaluated for the DNA lytic buffer (DALB) reformulation. As described in the Examples, numerous solution pathways were examined to address the challenge of developing storage-stable lysis buffers.

Disclosed herein include compositions and methods for mitigating precipitate formation which include or exclude one or more of the following approaches: (i) replacing an anionic surfactant with a non-ionic surfactant; (ii) supplementing the lysis buffer with additional metal ion(s); (iii) removing MgSO₄ from the lysis buffer; (iv) supplementing the lysis buffer with PEG or glycerol; (v) increasing glycine in the lysis buffer; (vi) employing an alternative counter ion (e.g., swap MgSO₄ for MgX); (vii) employing an alternative anionic surfactant; (viii) increasing (NH₄)₂SO₄ concentration; (ix) employing solvents (e.g., DMSO, alcohols); and (x) employing alternative surfactants to SDS (e.g., sodium decyl sulfate, sodium octyl sulfate).

In some embodiments, the methods and compositions provided herein do not comprise replacing an anionic surfactant with a non-ionic surfactant. Without being bound by any particular theory, while this approach could avoid charged interaction between Mg cation and SDS, it is unlikely that a non-ionic surfactant will have sufficient lytic power and matrix tolerance.

In some embodiments, the methods and compositions provided herein do not comprise supplementing the lysis buffer with additional metal ion. Without being bound by any particular theory, while this approach could provide SDS with alternative to Mg ion by selecting a metal whose complex with SDS is less prone to precipitation (such as cobalt (II) sulfate), based on data suggesting a 4-fold reduction in SDS is needed to mitigate precipitation, it is expected a significant amount of supplemental ion required, which carries with it a risk of assay impact.

In some embodiments, the methods and compositions provided herein do not comprise removing MgSO₄ from the lysis buffer. Without being bound by any particular theory, while this approach could thereby prevent the Mg-SDS interaction, there is a high risk to lyophilizing MgSO₄ with enzyme.

In some embodiments, the methods and compositions provided herein do not comprise adding EGTA or EDTA to chelate Mg ions. In some embodiments, the lysis buffer further comprises a metal chelator (e.g., EDTA or EGTA) to chelate divalent and trivalent cations such as zinc, manganese, nickel, copper, cobalt ions etc, which are cofactors of many enzymes including nucleases and proteases. Without being bound by any particular theory, while Mg ions must be available to enzyme during reaction, other divalent and trivalent cations can have deleterious effects on APA, so chelation must be very well balanced at function of pH and the functionality of Mg ion for subsequent amplification.

In some embodiments, the methods and compositions provided herein do not comprise supplementing the lysis buffer with PEG or glycerol. Without being bound by any particular theory, while the solvents may help SDS solubility, PEG presents viscosity concerns and glycerol presents hydrophobicity concerns.

In some embodiments, the methods and compositions provided herein do not comprise increasing glycine in the lysis buffer. Without being bound by any particular theory, this approach could employ the fact that glycine is weak chelator for Mg at low pH.

Compositions, kits, and methods for nucleic acid detection wherein lytic agents employed to lyse biological entities (e.g., viral particles, bacteria) are prevented from inactivating amplification reagents (e.g., polymerases) and wherein the deleterious activity of ribonucleases is inhibited at some or all stages are described in PCT Patent Application Publication No. WO2022198086A1, the content of which is incorporated herein by reference in its entirety. In some embodiments, the lysis buffers provided herein comprise one or more reducing agents (e.g., dithiothreitol (DTT)) as described therein and/or the reagent compositions provided herein comprise one or more protectants (e.g., a cyclodextrin compound) as described therein. In some embodiments, the disclosed methods and compositions enable isothermal amplification and real-time detection of nucleic acids without a need for sample separation or purification for point-of-care molecular diagnostics for direct pathogen lysis in clinical samples. There is provided, in some embodiments, a lysis buffer containing a potent ionic detergent that can be used to lyse pathogens in clinical samples. The amplification reagents can comprise a protectant against the lysis reagent, and can be dried (e.g., lyophilized, heat dried) and used for the amplification of the released nucleic acids in point-of-care settings.

The methods and compositions provided herein can be applied to other amplification methods for sample preparation without purification or separation, for example, PCR, RT-PCR, or other isothermal amplification methods. The methods and compositions provided herein can also be applied to genome sequencing methods or any nucleic acids (DNA or RNA) amplification or detection methods that require a sample preparation step. The methods and compositions provided herein can also find use in genotyping, diagnostics and forensics. The disclosed methods and compositions are not limited to isothermal amplification methods, but rather can be applied to other amplification/detection methods, for example, RT-PCR, WGS sequencing, and can also be applied to RNA purification/extraction without separation.

There are provided, in some embodiments, storage-stable lysis buffers. The term “storage-stable” as used herein shall be given its ordinary meaning, and shall also be used to describe a lysis buffer having a shelf-life acceptable for a product in the distribution chain of commerce, for instance, at least 12 months at a given temperature, and preferably, at least 24 months at a given temperature. The lysis buffer can be a precipitation-stable aqueous composition. Sufficient stability includes stability in storage such that after extended periods of time (e.g. 6 months, 12 months, 18 months, and 24 months) the lysis buffer can still be used to in a DNA and/or RNA assay described herein without a meaningful decrease in signal compared to an assay wherein the same lysis buffer is freshly prepared. In some embodiments, the lysis buffer comprises: one or more surfactants; (NH₄)₂SO₄; and MgSO₄, wherein the lysis buffer does not comprise SDS, CTAB, or both, and wherein formation of a precipitate is substantially inhibited for a period of time under a storage condition. In some embodiments, the appearance of the precipitate in the lysis buffer does not occur for at least about twenty days during the storage condition. In some embodiments, the lysis buffer comprises: one or more surfactants; (NH₄)₂SO₄; and MgSO₄, wherein formation of a precipitate is substantially inhibited for a period of time under a storage condition, and wherein the appearance of the precipitate in the lysis buffer does not occur for at least about twenty days during the storage condition. In some embodiments, the lysis buffer does not comprise one or more of SDS and CTAB.

In some embodiments, precipitate can be or can comprise a complex of Mg⁺² and the one or more surfactants. The lysis buffer can be a precipitation-stable aqueous composition. The precipitation of complexes consisting of Mg⁺² and the surfactant can be suppressed. The precipitation of complexes consisting of Mg⁺² and the surfactant can be eliminated. In some embodiments, appearance of the precipitate in the lysis buffer does not occur for at least about 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 22 months, 28 months, 32 months, 36 months, or a number or a range between any two of these values, during the storage condition.

The storage condition can comprise transport of the lysis buffer. The storage condition can comprise thermal stress, one or more freeze-thaw cycles, agitation, pressure changes, light irradiation, or any combination thereof. The storage condition can comprise ambient conditions (e.g., in the range from about 20° C. to about 25° C.). The storage condition can comprise refrigeration conditions (e.g., about 4° C.). The storage condition can comprise 14° C. The period of time can be at least about 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 22 months, 28 months, 32 months, 36 months, or a number or a range between any two of these values.

The MgSO₄ can be present in the lysis buffer at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, or a number or a range between any two of these values. The MgSO₄ can be present at a concentration of about 4 mM. In some embodiments, the concentration of soluble Mg⁺² is not reduced more than about 1.1-fold, 1.2-fold, 1.3-fold, 1.4-fold, 1.5-fold, 1.6-fold, 1.7-fold, 1.8-fold, 1.8-fold, 1.9-fold, 2-fold, 3-fold, 4-fold, 5-fold, 6-fold, 7-fold, 8-fold, 9-fold, 10-fold, 20-fold, or a number or a range between any two of these values, relative to the start of the period of time. Methods of measuring soluble Mg⁺² concentration are known to those skilled in the art, and include the those described in Example 1.

The amount of precipitation or homogeneity of the lysis buffer can be measured using various methods. In some embodiments, precipitation is monitored by using light scattering. In some embodiments, precipitation is monitored using a turbidity sensor, turbidimeter or nephelometer. In some embodiments, precipitation is monitored using a spectrophotometer. For example, it can be measured quantitatively using light scattering by illuminating the lysis buffer with a spectrophotometer. Or alternatively, the homogeneity can be measured qualitatively by observing the visual clarity of the solution with the eye. The substantial inhibition of formation of the precipitate can comprise the lysis buffer having no visible particulates as assessed by visual inspection. The appearance of the precipitate in the lysis buffer can comprise the lysis buffer having visible particulates as assessed by visual inspection.

The substantial inhibition of formation of the precipitate can comprise the lysis buffer having an absorbance at a wavelength below a threshold absorbance as determined by spectrophotometric analysis. The appearance of the precipitate in the lysis buffer can comprise the lysis buffer having an absorbance at a wavelength above a threshold absorbance as determined by spectrophotometric analysis. The threshold absorbance can be about 0.001, about 0.005, about 0.01, about 0.02, about 0.03, about 0.04, about 0.05, about 0.06, about 0.07, about 0.08, about 0.09, about 0.1, about 0.2, about 0.4, about 0.6, about 0.8, about 1.0, about 2.0, about 4.0, about 6.0, or a number or a range between any two of these values, absorbance units (AU). The appearance of the precipitate in the lysis buffer can comprise absorbance at a wavelength greater than about 1.1-fold, 1.2-fold, 1.3-fold, 1.4-fold, 1.5-fold, 1.6-fold, 1.7-fold, 1.8-fold, 1.8-fold, 1.9-fold, 2-fold, 3-fold, 4-fold, 5-fold, 6-fold, 7-fold, 8-fold, 9-fold, 10-fold, 20-fold, or a number or a range between any two of these values, the absorbance at said wavelength at the start of the period of time as determined by spectrophotometric analysis. The substantial inhibition of formation of the precipitate can comprise the lysis buffer having an absorbance at a wavelength less than about 1.1-fold, 1.2-fold, 1.3-fold, 1.4-fold, 1.5-fold, 1.6-fold, 1.7-fold, 1.8-fold, 1.8-fold, 1.9-fold, 2-fold, 3-fold, 4-fold, 5-fold, 6-fold, 7-fold, 8-fold, 9-fold, 10-fold, 20-fold, or a number or a range between any two of these values, the absorbance at said wavelength at the start of the period of time as determined by spectrophotometric analysis. The spectrophotometric analysis can be conducted with a spectrophotometer at a wavelength (or a wavelength range) of about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550 nm, about 560 nm, about 570 nm, about 580 nm, about 590 nm, about 600 nm, about 610 nm, about 620 nm, about 630 nm, about 640 nm, about 650 nm, about 660 nm, about 670 nm, about 680 nm, about 690 nm, about 700 nm, about 710 nm, about 720 nm, about 730 nm, about 740 nm, about 750 nm, about 760 nm, about 770 nm, about 780 nm, about 790 nm, about 800 nm, about 810 nm, about 820 nm, about 830 nm, about 840 nm, about 850 nm, about 860 nm, about 870 nm, about 880 nm, about 890 nm, about 900 nm, or a number or a range between any two of these values. The wavelength can be the wavelength that maximizes absorbance of the precipitate. In some embodiments, the spectrophotometric analysis is conducted at a wavelength range that exhibits the greatest absorbance for the precipitate. The absorbance value can be due to light scattering of the precipitate. The spectrophotometer can be selected from the group consisting of a UV-Vis spectrophotometer, an IR spectrophotometer, a Visible-Near IR spectrophotometer and a Raman spectrophotometer

The lysis buffer further can comprise one or more alcohols. The alcohol(s) present in the lysis buffers provided herein can vary depending on the embodiment. For example, the carbon chain length of the alcohol can vary. For example, in some embodiments, the alcohol is at least, or is at most, 1, 2, 3, 4, 5, or 6, carbons in length. The alcohols are not particularly limited, and can be cyclic or acyclic, or saturated or unsaturated. The one or more alcohols can increase surfactant solubility. The one or more alcohols can be selected from the group comprising ethanol, isopropanol, isobutyl alcohol, pentanol, and hexanol. Alcohols contemplated for use with the lysis buffers provided herein include, but are not limited to, methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, 1-heptanol, 2-heptanol, 3-heptanol, 1-octanol, 2-octanol, 2-ethyl-1-hexanol, 1-nonanol, 1-decanol, 1-undecanol, 1-dodecanol, allyl alcohol, propargyl alcohol, benzyl alcohol, cyclohexanol, 1-methylcyclohexanol, 2-methylcyclohexanol, 3-methylcyclohexanol, 4-methylcyclohexanol, 1,2-ethanediol, 1,2-propandiol, 1,3-propandiol, 1,2-butanediol, 1,3-butanediol, 1,4-butanediol, 2,3-butanediol, 1,5-pentanediol, and glycerol. The one or more alcohols can comprise about 0.001%, 0.01%, 0.1%, 1%, 2%, 3%, 4%, 5%, or a number or a range between any two of these values, (v/v) of the lysis buffer. The lysis buffer can further comprise a reducing agent. The reducing agent can present at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 60 mM, 70 mM, 80 mM, 90 mM, 100 mM, or a number or a range between any two of these values. In some embodiments, the reducing agent is or comprises cysteine.

The (NH₄)₂SO₄ can be present at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, or a number or a range between any two of these values. The (NH₄)₂SO₄ can be present at a concentration of about 5 mM or about 10 mM, and the appearance of the precipitate in the lysis buffer is delayed by at least about 2 days, 1 week, 2 weeks, 3 weeks, 1 month, 3 months, 6 months, 1 year, 2 years, or a number or a range between any two of these values, as compared a comparable lysis buffer wherein the (NH₄)₂SO₄ is present at a concentration of about 5 mM.

The lysis buffer can comprise one or more acids. The acid(s) present in the lysis buffers provided herein can vary depending on the embodiment. The acid can comprise an organic acid, an inorganic acid, or a mixture thereof. The inorganic acid can be hydrogen chloride (HCl). The inorganic acid can comprise one or more of hydrochloric acid, nitric acid, nitrous acid, phosphoric acid, phosphinic acid, phosphonic acid, sulfonic acid, sulfuric acid, sulfurous acid, and boric acid. The organic acid can comprise one or more of acetic acid, C₂H₅COOH, C₃H—COOH, C₄H₉COOH, (COOH)₂, CH₂ (COOH)₂, C₂H₄ (COOH)₂, C₃H₆ (COOH)₂, C₄H₅(COOH)₂, C₅H₁₀(COOH)₂, fumaric acid, maleic acid, malonic acid, lactic acid, citric acid, tartaric acid, oxalic acid, ascorbic acid, benzoic acid, salicylic acid, phthalic acid, pyruvic acid, L-aspartic acid, D-aspartic acid, carbonic acid, formic acid, methanesulfonic acid, ethanesulfonic acid, benzenesulfonic acid, glucosamine sulphate, L-threonic acid, camphoric acid, gluconic acid, L-glutamic acid, D-glutamic acid, trifluoroacetic acid or ranelic acid. The acid can be present in the lysis buffer at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, or a number or a range between any two of these values.

The lysis buffer can comprise: a pH buffer (e.g., buffering agent). The pH buffer can be present in the lysis buffer at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, or a number or a range between any two of these values. The buffering agent(s) present in the lysis buffers provided herein can vary depending on the embodiment, and include citrate buffer, maleate, phosphate, glycine, glycylglycine, malate, succinate, carbonate, ethanolamine, DIPSO, ADA, imidazole, hydrazine, HEPBS, MES, MOBS, PIPES, EPPS, TAPS, TABS, borate, taurine, N-(2-Acetamido)-aminoethanesulfonic acid (ACES), Salt of acetic acid (Acetate), N-(2-Acetamido)-iminodiacetic acid (ADA), 2-Aminoethanesulfonic acid, Taurine (AES), Ammonia, 2-Amino-2-methyl-1-propanol (AMP), 2-Amino-2-methyl-1,3-propanediol, (Ammediol or AMPD), N-(1,1-Dimethyl-2-hydroxyethyl)-3-amino-2-hydroxypropanesulfonic acid (AMPSO), N,N-Bis-(2-hydroxyethyl)-2-aminoethanesulfonic acid (BES), Sodium Bicarbonate, N,N′-Bis(2-hydroxyethyl)-glycine (Bicine), [Bis-(2-hydroxyethyl)-imino]-tris-(hydroxymethylmethane) (BIS-Tris), 1,3-Bis [tris(hydroxymethyl)-methylamino]propane) (BIS-Tris-Propane), Boric acid, Dimethylarsinic acid (Cacodylate), 3-(Cyclohexylamino)-propanesulfonic acid (CAPS), 3-(Cyclohexylamino)-2-hydroxy-1-propanesulfonic acid (CAPSO), Sodium carbonate, Cyclohexylaminoethanesulfonic acid (CHES), Salt of acid citric (Citrate), 3-[N-Bis(hydroxyethyl)amino]-2-hydroxypropanesulfonic acid (DIPSO), Formate Salt of formic acid, Glycine, Glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), Imidazole, Salt of malic acid (Malate), Maleate Salt of maleic acid, 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), Salt of phosphoric acid (Phosphate), Piperazine-N,N′-bis(2-ethanesulfonic acid) (PIPES), Piperazine-N,N′-bis(2-hydroxypropanesulfonic acid) (POPSO), Pyridine, Salt of succinic acid (Succinate), 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), Triethanolamine (TEA), 2-[Tris(hydroxymethyl)-methylamino]-ethanesulfonic acid (TES), N-[Tris(hydroxymethyl)-methyl]-glycine (Tricine), and Tris(hydroxymethyl)-aminomethane (Tris), or any combination thereof. The pH buffer can comprise glycine. The pH buffer can comprise glycine-HCl. The pH buffer can comprise glycine at about 0.1 mM to 50 mM. The pH buffer can comprise 10.0 mM glycine. The pH buffer can comprise HCl at 0 mM to about 20 mM. The pH of the lysis buffer can be about 1.0 to about 6.0. The pH of the lysis buffer can be at least about 1, 2, 3, 4, 5, 6, 7, 8, 9, or 10 or more. The pH of the lysis buffer can be at most about 1, 2, 3, 4, or 5. The pH of the lysis buffer can be about 2.2. The concentration of glycine can determine the buffer capacity of glycine-HCL and the ratio of glycine/HCl can determine the buffer pH.

In some embodiments, the percentages of lysis buffer components disclosed herein are provided as % w/w, % m/v, % v/v, % m/w, % w/v, or variations thereof. In some embodiments, the percentage (% w/w, % m/v, % v/v, % m/w, % w/v, or variations thereof) of the lysis buffer components disclosed herein (e.g., one or more surfactants, (NH₄)₂SO₄, MgSO₄, acid(s), alcohols, pH buffer(s), tween surfactant(s)) within the lysis buffer can be, or be about, 0.000000001%, 0.00000001%, 0.0000001%, 0.000001%, 0.00001%, 0.0001%, 0.001%, 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.0%, 1.01%, 1.02%, 1.03%, 1.04%, 1.05%, 1.06%, 1.07%, 1.08%, 1.09%, 1.10%, 1.11%, 1.12%, 1.13%, 1.14%, 1.15%, 1.16%, 1.17%, 1.18%, 1.19%, 1.20%, 1.21%, 1.22%, 1.23%, 1.24%, 1.25%, 1.26%, 1.27%, 1.28%, 1.29%, 1.30%, 1.31%, 1.32%, 1.33%, 1.34%, 1.35%, 1.36%, 1.37%, 1.38%, 1.39%, 1.40%, 1.41%, 1.42%, 1.43%, 1.44%, 1.45%, 1.46%, 1.47%, 1.48%, 1.49%, 1.50%, 1.51%, 1.52%, 1.53%, 1.54%, 1.55%, 1.56%, 1.57%, 1.58%, 1.59%, 1.60%, 1.61%, 1.62%, 1.63%, 1.64%, 1.65%, 1.66%, 1.67%, 1.68%, 1.69%, 1.70%, 1.71%, 1.72%, 1.73%, 1.74%, 1.75%, 1.76%, 1.77%, 1.78%, 1.79%, 1.80%, 1.81%, 1.82%, 1.83%, 1.84%, 1.85%, 1.86%, 1.87%, 1.88%, 1.89%, 1.90%, 1.91%, 1.92%, 1.93%, 1.94%, 1.95%, 1.96%, 1.97%, 1.98%, 1.99%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values In some embodiments, the percentages of lysis buffer components disclosed herein (e.g., one or more surfactants, (NH₄)₂SO₄, MgSO₄, acid(s), alcohols, pH buffer(s), tween surfactant(s)) are provided as % w/w, % m/v, % v/v, % m/w, % w/v, or variations thereof. In some embodiments, the percentages of lysis buffer components disclosed herein are described with regards to their final concentration once the lysis buffer is contacted with a sample comprising biological entities. Additionally, while in some embodiments, the lysis buffer components disclosed herein are described in with regards to working concentrations (e.g., 1×) of the lysis buffer, the disclosure also contemplates concentrated versions of the disclosed lysis buffers (e.g., a 2× lysis buffer).

The one or more surfactants can comprise about 0.001% (w/v) to about 2.0% (w/v) of the lysis buffer. The one or more surfactants can be capable of lysing biological entities to release sample nucleic acids comprised therein. The sample nucleic acids can comprise sample ribonucleic acids and/or sample deoxyribonucleic acids. The biological entities can comprise a virus, a bacteria, a fungi, a protozoa, portions thereof, or any combination thereof. The biological entities can comprise one or more of prokaryotic cells, eukaryotic cells, viral particles, exosomes, protoplasts, and microvesicles. The one or more surfactants can comprise about 0.001%, 0.01%, 0.1%, 1%, 2%, 3%, 4%, 5%, 6%, 7%, 8%, 9%, 10%, or a number or a range between any two of these values, (w/v) of the lysis buffer. The one or more surfactants disclosed herein can comprise one or more of a cationic surfactant, an anionic surfactant, a non-ionic surfactant, and an amphoteric surfactant. The lysis buffer further can comprise a tween surfactant. The tween surfactant can be selected from the group consisting of Tween 20, Tween 40, Tween 45, Tween 60, Tween 65, Tween 80, Tween 81 and Tween 85. The tween surfactant can comprise about 0.01% (w/v) to about 1.0% (w/v) of the lysis buffer.

The one or more surfactants can comprise CTAB. In some embodiments, the lysis buffer comprises about 0.2% (w/v) CTAB and the (NH₄)₂SO₄ is present at a concentration of about 5 mM. The one or more surfactants can comprise CTAC. In some embodiments, the lysis buffer comprises about 0.2% (w/v) CTAC and the (NH₄)₂SO₄ is present at a concentration of about 5 mM. The one or more surfactants can comprise SDS. In some embodiments, the lysis buffer comprises about 0.4% (w/v) SDS and the (NH₄)₂SO₄ is present at a concentration of about 10 mM. In some embodiments, the lysis buffer further comprises Tween 80. The one or more surfactants can comprise SDeS. In some embodiments, the lysis buffer comprises about 0.2% (w/v) SDeS and the (NH₄)₂SO₄ is present at a concentration of about 5 mM. The one or more surfactants can comprise SDeS. In some embodiments, the lysis buffer comprises about 0.2% (w/v) SDeS and the (NH₄)₂SO₄ is present at a concentration of about 10 mM. In some embodiments, the lysis buffer comprises about 0.4% (w/v) SDeS and the (NH₄)₂SO₄ is present at a concentration of about 10 mM. The one or more surfactants can comprise SDeS. The lysis buffer can, for example, comprise about 0.8% (w/v) SDeS and the (NH₄)₂SO₄ is present at a concentration of about 10 mM, or about 0.4% (w/v) SDeS and the (NH₄)₂SO₄ is present at a concentration of about 10 mM. In some embodiments, the lysis buffer further comprises Tween 80. In some embodiments, the lysis buffer comprises about 0.2% (w/v) CTAB and the (NH₄)₂SO₄ is present at a concentration of about 5 mM. In some embodiments, the lysis buffer comprises about 0.2% (w/v) CTAC and the (NH₄)₂SO₄ is present at a concentration of about 5 mM. The one or more surfactants can comprise sodium octyl sulfate (S Octyl S). In some embodiments, the lysis buffer comprises about 0.2% (w/v) S Octyl S and the (NH₄)₂SO₄ is present at a concentration of about 5 mM.

Disclosed herein include methods for detecting a target nucleic acid sequence in a sample. In some embodiments, the method comprises: (a) contacting a sample comprising biological entities with a lysis buffer provided herein to generate a treated sample, wherein the lysis buffer is capable of lysing biological entities to release sample nucleic acids comprised therein, wherein the sample nucleic acids are suspected of comprising a target nucleic acid sequence. The method can comprise: (b) contacting a reagent composition (e.g., a wet composition, a dried composition) with the treated sample to generate an amplification reaction mixture, wherein the reagent composition comprises one or more amplification reagents. The method can comprise: (c) amplifying a target nucleic acid sequence in the amplification reaction mixture, thereby generating a nucleic acid amplification product. The method can comprise: (d) detecting the nucleic acid amplification product, wherein the detecting is performed in less than about 20 minutes (e.g., about 20, 19, 18, 17, 16, 15, 14, 13, 12, 11, 10, 9, 8, 7, 6, 5, 4, 3, 2, 1 minute(s), or a number or a range between any two of these values) from the time the reagent composition is contacted with the treated sample. In some embodiments, steps (b) and (c) are performed concurrently (e.g., the amplification begins once the contacting of the reagent composition and the treated sample has occurred). In some embodiments, the reagent composition comprises two or more dried compositions (comprising the same or different components) or two or more wet compositions (comprising the same or different components). In some embodiments, the lysis buffer comprises two or more lysis buffers (comprising the same or different components).

The sample nucleic acids can comprise sample ribonucleic acids and/or sample deoxyribonucleic acids. The sample ribonucleic acids can comprise a cellular RNA, a mRNA, a microRNA, a bacterial RNA, a viral RNA, or any combination thereof. The one or more amplification reagents can comprise a reverse transcriptase and/or an enzyme having a hyperthermophile polymerase activity. In some embodiments, the enzyme having a hyperthermophile polymerase activity has a reverse transcriptase activity. Contacting the reagent composition with the treated sample can comprise dissolving the reagent composition in the treated sample. The reagent composition can comprise one or more of a reverse transcriptase, an enzyme having a hyperthermophile polymerase activity, a first primer, a second primer, and a reverse transcription primer. The amplifying can be performed in an isothermal amplification condition. Detecting the nucleic acid amplification product can comprise use of a real-time detection method.

The one or more lytic reagents (e.g., one or more surfactants) can comprise about 0.001% (w/v) to about 1.0% (w/v) (e.g., about 0.2% (w/v)) of the treated sample. The sample nucleic acids can comprise a nucleic acid comprising the target nucleic acid sequence. The target nucleic acid sequence can comprise a first strand and a second strand complementary to each other.

Amplifying the target nucleic acid sequence can comprise: amplifying a target nucleic acid sequence comprising a first strand and a second strand complementary to each other in an isothermal amplification condition, wherein the amplifying comprises contacting a nucleic acid comprising the target nucleic acid sequence with: i) a first primer and a second primer, wherein the first primer is capable of hybridizing to a sequence of the first strand of the target nucleic acid sequence, and the second primer is capable of hybridizing to a sequence of the second strand of the target nucleic acid sequence; and ii) an enzyme having a hyperthermophile polymerase activity, thereby generating a nucleic acid amplification product, wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

In some embodiments, the amplifying does not comprise using any enzyme other than the enzyme having a hyperthermophile polymerase activity, and the amplifying step does not comprise denaturing the nucleic acid. In some embodiments, the method does not comprise contacting the nucleic acid with a single-stranded DNA binding protein prior to or during step (c). In some embodiments, the method does not comprise thermal or enzymatic denaturation of the sample nucleic acid.

The nucleic acid can be a double-stranded DNA. The nucleic acid can be a product of reverse transcription reaction. The nucleic acid can be a product of reverse transcription reaction generated from sample ribonucleic acids. Step (c) can comprise generating the nucleic acid by a reverse transcription reaction.

The sample nucleic acids can comprise sample ribonucleic acids. The method can comprise contacting sample ribonucleic acids with a reverse transcriptase and/or a reverse transcription primer to generate a cDNA. Amplifying the target nucleic acid sequence can comprise: (c1) contacting sample ribonucleic acids with a reverse transcriptase and/or a reverse transcription primer to generate a cDNA; (c2) contacting the cDNA with an enzyme having a hyperthermophile polymerase activity to generate a double-stranded DNA (dsDNA), wherein the dsDNA comprises a target nucleic acid sequence, and wherein the target nucleic acid sequence comprises a first strand and a second strand complementary to each other; (c3) amplifying the target nucleic acid sequence under an isothermal amplification condition, wherein the amplifying comprises contacting the dsDNA with: (i) a first primer and a second primer, wherein the first primer is capable of hybridizing to a sequence of the first strand of the target nucleic acid sequence, and the second primer is capable of hybridizing to a sequence of the second strand of the target nucleic acid sequence; and (ii) the enzyme having a hyperthermophile polymerase activity, thereby generating a nucleic acid amplification product, wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

In some embodiments, the method does not comprise using any enzymes other than the reverse transcriptase and the enzyme having a hyperthermophile polymerase activity. Step (d) further can comprise determining the amount of the dsDNA and/or nucleic acid that comprises the target nucleic acid sequence in the sample. In some embodiments, the enzyme having a hyperthermophile polymerase activity has an amino acid sequence that is at least about 90% or 95% identical to the amino acid sequence of SEQ ID NO: 1 or a functional fragment thereof. The enzyme having a hyperthermophile polymerase activity can be a polymerase comprising the amino acid sequence of SEQ ID NO: 1. In some embodiments, the enzyme having a hyperthermophile polymerase activity has low or no exonuclease activity.

Amplifying the target nucleic acid sequence can be performed at a constant temperature of about 55° C. to about 75° C., for example about 65° C. The first primer, the second primer, or both can be about 8 to 16 bases long. The first primer, the second primer, or both can comprise one or more of DNA bases, modified DNA bases, or a combination thereof. The nucleic acid amplification product can be about 20 to 40 bases long. The spacer sequence can comprise a portion of the target nucleic acid sequence. The spacer sequence can be 1 to 10 bases long.

In some embodiments, the method comprises: contacting the nucleic acid amplification product with a signal-generating oligonucleotide capable of hybridizing to the amplification product. The signal-generating oligonucleotide can comprise a fluorophore, a quencher, or both. Detecting the nucleic acid amplification product can comprise detecting a fluorescent signal. The fluorescent signal can be from a molecular beacon. The method can be performed in a single reaction vessel. The sample ribonucleic acids can be contacted with the reverse transcriptase and the enzyme having a hyperthermophile polymerase activity simultaneously. The sample ribonucleic acids can be contacted with the reverse transcriptase, the enzyme having a hyperthermophile polymerase activity, and the first and second primers simultaneously. In some embodiments, the sample ribonucleic acids are contacted with the reverse transcriptase, the enzyme having a hyperthermophile polymerase activity, the first primer, the second primer, and the reverse transcription primer simultaneously. Reverse transcription of the sample ribonucleic acids can occur by the addition of a reverse transcription primer. In some embodiments, the reverse transcription primer is an oligo (dT) primer, random hexanucleotide primer, or a target-specific oligonucleotide primer. In some embodiments, oligo (dT) primers are 12-18 nucleotides in length and bind to the endogenous poly(A)+ tail at the 3′ end of mRNA. Random hexanucleotide primers can bind to sample ribonucleic acids at a variety of complementary sites. Target-specific oligonucleotide primers typically selectively prime the sample ribonucleic acids of interest. In some embodiments, the first primer and/or second primer is a reverse transcription primer.

The amplifying step can comprise multiplex amplification of two or more target nucleic acid sequences. The detecting step can comprise multiplex detection of two or more nucleic acid amplification products derived from said two or more target nucleic acid sequences. The two or more target nucleic acid sequences can be specific to two or more different organisms. The two or more different organisms can comprise one or more of SARS-COV-2, Influenza A, Influenza B, and/or Influenza C.

The lysis buffers provided herein can be employed upstream of a variety of amplification reactions, such as, for example, isothermal amplification reactions. In some embodiments, the amplification comprises one or more of the following amplification methods: APA, LAMP, HDA, RPA, SDA, NASBA, TMA, NEAR, RCA, MDA, RAM, cHDA, SPIA, SMART, 3SR, GEAR and IMDA. In some embodiments, the amplifying does not comprise one or more of the following amplification methods: APA, LAMP, HDA, RPA, SDA, NASBA, TMA, NEAR, RCA, MDA, RAM, CHDA, SPIA, SMART, 3SR, GEAR and IMDA. In some embodiments, the amplifying does not comprise LAMP.

In some embodiments, the method does not comprise one or more, or any, of the following: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of ribonuclease inhibitors to the treated sample; (vii) the addition of ribonuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heat and/or enzymatic denaturing the sample nucleic acids prior to or during amplification; and (xiii) the addition of ribonuclease H to the treated sample or amplification reaction mixture. In some embodiments, the sample is held at an amplification temperature (e.g., 67° C.). In some embodiments, the sample (e.g. a sample comprising RNA) is held at temperature between room temperature and reaction temperature for 1-2 minutes before the amplification reaction to facilitate reverse transcription reaction.

In some embodiments, step (a), step (b), step (c), and/or step (d) is performed for a period of about 20 minutes, about 15 minutes, about 10 minutes, about 5 minutes, about 2.5 minutes, or about 1 minute. In some embodiments, step (a), step (b), step (c), and/or step (d) comprises sonication, osmotic shock, chemical treatment, heating, or any combination thereof.

The term “isothermal amplification reaction” shall be given its ordinary meaning and also include reactions wherein the temperature does not significantly change during the reaction. In some embodiments, the temperature of the isothermal amplification reaction does not deviate by more than 10° C., for example by not more than 5° C. and by not more than 2° C. during the main enzymatic reaction step where amplification takes place. Depending on the method of isothermal amplification of nucleic acids, different enzymes can be used for amplification. Isothermal amplification compositions and methods are described in WO2017176404, the content of which is incorporated herein by reference in its entirety.

Disclosed herein include methods for amplifying nucleic acids. In some embodiments, the method comprises: contacting sample nucleic acid under isothermal amplification conditions with components comprising a) at least one oligonucleotide, which at least one oligonucleotide comprises a polynucleotide complementary to a target sequence in the sample nucleic acid, and b) at least one component providing hyperthermophile polymerase activity, thereby generating a nucleic acid amplification product. In some embodiments, the method comprises: contacting sample nucleic acid under isothermal amplification conditions with a) non-enzymatic components comprising at least one oligonucleotide, which at least one oligonucleotide comprises a polynucleotide complementary to a target sequence in the sample nucleic acid, and b) an enzymatic component consisting of a hyperthermophile polymerase or a polymerase comprising an amino acid sequence that is at least about 90% identical to a hyperthermophile polymerase, thereby generating a nucleic acid amplification product. In some embodiments, the method comprises: contacting sample nucleic acid under isothermal amplification conditions with a) non-enzymatic components comprising at least one oligonucleotide, which at least one oligonucleotide comprises a polynucleotide complementary to a target sequence in the sample nucleic acid, and b) enzymatic activity consisting of i) hyperthermophile polymerase activity and, optionally, ii) reverse transcriptase activity, thereby generating a nucleic acid amplification product.

Disclosed herein include methods for processing nucleic acids. In some embodiments, the method comprises: amplifying nucleic acid, wherein the amplifying consists essentially of contacting sample nucleic acid under isothermal amplification conditions with a) at least one oligonucleotide, which at least one oligonucleotide comprises a polynucleotide complementary to a target sequence in the sample nucleic acid, and b) at least one component providing hyperthermophile polymerase activity, thereby generating a nucleic acid amplification product. In some embodiments, the method for processing nucleic acids comprises: amplifying nucleic acid, wherein the amplifying consists essentially of contacting sample nucleic acid under isothermal amplification conditions with a) non-enzymatic components comprising at least one oligonucleotide, which at least one oligonucleotide comprises a polynucleotide complementary to a target sequence in the sample nucleic acid, and b) an enzymatic component consisting of a hyperthermophile polymerase or a polymerase comprising an amino acid sequence that is at least about 90% identical to a hyperthermophile polymerase, thereby generating a nucleic acid amplification product. In some embodiments, the method for processing nucleic acids comprises: amplifying nucleic acid, wherein the amplifying consists essentially of contacting sample nucleic acid under isothermal amplification conditions with a) non-enzymatic components comprising at least one oligonucleotide, which at least one oligonucleotide comprises a polynucleotide complementary to a target sequence in the sample nucleic acid, and b) enzymatic activity consisting of i) hyperthermophile polymerase activity and, optionally, ii) reverse transcriptase activity, thereby generating a nucleic acid amplification product. In some embodiments, the enzymatic activity consists of i) hyperthermophile polymerase activity, and ii) reverse transcriptase activity. In some embodiments, the method comprises: amplifying nucleic acid, wherein the amplifying consists of contacting sample nucleic acid under isothermal amplification conditions with a) at least one oligonucleotide, which at least one oligonucleotide comprises a polynucleotide complementary to a target sequence in the sample nucleic acid, and b) at least one component providing hyperthermophile polymerase activity, thereby generating a nucleic acid amplification product. In some embodiments, the method for processing nucleic acids comprises: amplifying nucleic acid, wherein the amplifying consists of contacting sample nucleic acid under isothermal amplification conditions with a) non-enzymatic components comprising at least one oligonucleotide, which at least one oligonucleotide comprises a polynucleotide complementary to a target sequence in the sample nucleic acid, and b) an enzymatic component consisting of a hyperthermophile polymerase or a polymerase comprising an amino acid sequence that is at least about 90% identical to a hyperthermophile polymerase, thereby generating a nucleic acid amplification product. In some embodiments, the method for processing nucleic acids comprises: amplifying nucleic acid, wherein the amplifying consists of contacting sample nucleic acid under isothermal amplification conditions with a) non-enzymatic components comprising at least one oligonucleotide, which at least one oligonucleotide comprises a polynucleotide complementary to a target sequence in the sample nucleic acid, and b) enzymatic activity consisting of i) hyperthermophile polymerase activity and, optionally, ii) reverse transcriptase activity, thereby generating a nucleic acid amplification product.

Disclosed herein include methods for determining the presence, absence or amount of a target sequence in sample nucleic acid. In some embodiments, the method comprises: a) amplifying a target sequence in the sample nucleic acid, wherein: the target sequence comprises a first strand and a second strand, the first strand and second strand are complementary to each other, and the amplifying comprises contacting sample nucleic acid under helicase-free isothermal amplification conditions with: i) a first oligonucleotide and a second oligonucleotide, wherein the first oligonucleotide comprises or consists of a first polynucleotide continuously complementary to a sequence in the first strand, and the second oligonucleotide comprises or consists of a second polynucleotide continuously complementary to a sequence in the second strand; and ii) at least one component providing a hyperthermophile polymerase activity, thereby generating a nucleic acid amplification product, wherein the nucleic acid amplification product comprises or consists of 1) a first nucleotide sequence that is continuously complementary to or substantially identical to the first polynucleotide of the first oligonucleotide, 2) a second nucleotide sequence that is continuously complementary to or substantially identical to the second polynucleotide of the second oligonucleotide, and 3) a spacer sequence comprising 1 to 10 bases, and the spacer sequence is flanked by the first nucleotide sequence and the second nucleotide sequence; and b) detecting the nucleic acid amplification product, wherein detecting the nucleic acid amplification product comprises use of a real-time detection method and is performed in 10 minutes or less from the time the sample nucleic acid is contacted with (a) (i) and (a) (ii), whereby the presence, absence or amount of a target sequence in sample nucleic acid is determined.

Disclosed herein include kits for determining the presence, absence or amount of a target sequence in sample nucleic acid. In some embodiments, the kit comprises: a) components for amplifying a target sequence in the sample nucleic acid under helicase-free isothermal amplification conditions, which components comprise: i) a first oligonucleotide and a second oligonucleotide, wherein the first oligonucleotide comprises or consists of a first polynucleotide continuously complementary to a sequence in a first strand of the target sequence, and the second oligonucleotide comprises or consists of a second polynucleotide continuously complementary to a sequence in a second strand of the target sequence, which first strand and second strand of the target sequence are complementary to each other; and ii) at least one component providing a hyperthermophile polymerase activity; and b) at least one component providing real-time detection activity for a nucleic acid amplification product.

The enzymatic activity can comprise, or consist of, one or more of the following: i) hyperthermophile polymerase activity, ii) reverse transcriptase activity, and iii) 3′ to 5′ exonuclease activity. In some embodiments, the method does not comprise enzymatic denaturation and/or heat denaturation of the sample nucleic acid prior to or during amplification. In some embodiments, the sample nucleic acid is not contacted with an endonuclease prior to or during amplification. In some embodiments, the sample nucleic acid is not contacted with an unwinding agent prior to or during amplification. In some embodiments, the sample nucleic acid is not contacted with a helicase prior to or during amplification. In some embodiments, the sample nucleic acid is not contacted with a recombinase prior to or during amplification. In some embodiments, the sample nucleic acid is not contacted with a single-stranded DNA binding protein prior to or during amplification. In some embodiments, the sample nucleic acid is unmodified prior to amplification. In some embodiments, the unmodified sample nucleic acid is from disrupted cells. In some embodiments, the sample nucleic acid comprises DNA. In some embodiments, the sample nucleic acid comprises genomic DNA. In some embodiments, the sample nucleic acid comprises RNA. In some embodiments, the sample nucleic acid comprises viral RNA. In some embodiments, the sample nucleic acid comprises bacterial RNA. The sample nucleic acid can comprise single-stranded nucleic acid, double-stranded nucleic acid, or both. For example, the double-stranded nucleic acid can comprise a first strand and a second strand. In some embodiments, the at least one oligonucleotide comprises a first oligonucleotide and a second oligonucleotide. In some embodiments, the at least one oligonucleotide consists of a first oligonucleotide and a second oligonucleotide. In some embodiments, the first oligonucleotide and the second oligonucleotide each comprise 8 to 16 bases. In some embodiments, the first oligonucleotide comprises a first polynucleotide complementary to a target sequence in the first strand of the sample nucleic acid, and the second oligonucleotide comprises a second polynucleotide complementary to a target sequence in the second strand of the sample nucleic acid. In some embodiments, the first oligonucleotide comprises a first polynucleotide continuously complementary to a target sequence in the first strand of the sample nucleic acid, and the second oligonucleotide comprises a second polynucleotide continuously complementary to a target sequence in the second strand of the sample nucleic acid. In some embodiments, the first oligonucleotide consists of a first polynucleotide continuously complementary to a target sequence in the first strand of the sample nucleic acid, and the second oligonucleotide consists of a second polynucleotide continuously complementary to a target sequence in the second strand of the sample nucleic acid. In some embodiments, sample nucleic acid is obtained from a subject prior to amplification. In some embodiments, unpurified sample nucleic acid is amplified. In some embodiments, purified sample nucleic acid is amplified. In some embodiments, the method further comprises purifying sample nucleic acid prior to amplification.

In some embodiments, the hyperthermophile polymerase activity is provided by a hyperthermophile polymerase or functional fragment thereof, or a polymerase comprising an amino acid sequence that is at least about 90% identical to a hyperthermophile polymerase or functional fragment thereof. In some embodiments, the hyperthermophile polymerase activity is provided by an Archaea hyperthermophile polymerase or functional fragment thereof. In some embodiments, the hyperthermophile polymerase activity is provided by a polymerase comprising an amino acid sequence of SEQ ID NO: 1 or functional fragment thereof. In some embodiments, the hyperthermophile polymerase activity is provided by a polymerase comprising an amino acid sequence that is at least about 90% identical to the amino acid sequence of SEQ ID NO: 1 or functional fragment thereof. In some embodiments, the hyperthermophile polymerase activity is provided by a polymerase having low exonuclease activity. In some embodiments, the hyperthermophile polymerase activity is provided by a polymerase having no exonuclease activity. In some embodiments, the amplification is performed at a constant temperature of about 55° C. to about 75° C., for example, a constant temperature of about 55° C. to about 65° C. or a constant temperature of about 65° C. or at a constant temperature of about 60° C.

In some embodiments, the nucleic acid amplification product is detectable in 10 minutes or less. In some embodiments, the nucleic acid amplification product comprises a polynucleotide that is continuously complementary to or substantially identical to a target sequence in the sample nucleic acid. In some embodiments, the nucleic acid amplification product consists of a polynucleotide that is continuously complementary to or substantially identical to a target sequence in the sample nucleic acid. In some embodiments, the nucleic acid amplification product is about 20 to 40 bases long. In some embodiments, the nucleic acid amplification product comprises i) a first nucleotide sequence that is continuously complementary to or substantially identical to the first polynucleotide of the first oligonucleotide, ii) a second nucleotide sequence that is continuously complementary to or substantially identical to the second polynucleotide of the second oligonucleotide, and iii) a spacer sequence, wherein the spacer sequence is flanked by the first nucleotide sequence and the second nucleotide sequence. In some embodiments, the nucleic acid amplification product consists of i) a first nucleotide sequence that is continuously complementary to or substantially identical to the first polynucleotide of the first oligonucleotide, ii) a second nucleotide sequence that is continuously complementary to or substantially identical to the second polynucleotide of the second oligonucleotide, and iii) a spacer sequence, wherein the spacer sequence is flanked by the first nucleotide sequence and the second nucleotide sequence. In some embodiments, the spacer sequence comprises 1 to 10 bases. In some embodiments, the spacer sequence comprises 1 to 5 bases. In some embodiments, the spacer sequence is not complementary to or identical to the first polynucleotide of the first oligonucleotide and is not complementary to or identical to the second polynucleotide of the second oligonucleotide. In some embodiments, the spacer sequence is continuously complementary to or substantially identical to a portion of a target sequence in the sample nucleic acid.

In some embodiments, the method further comprises detecting the nucleic acid amplification product. In some embodiments, detecting the nucleic acid amplification product is performed in 10 minutes or less from the time the sample nucleic acid is contacted with the component providing the hyperthermophile polymerase activity and the at least one oligonucleotide. In some embodiments, detecting the nucleic acid amplification product comprises use of a real-time detection method. In some embodiments, detecting the nucleic acid amplification product comprises detection of a fluorescent signal. In some embodiments, the fluorescent signal is from a molecular beacon. In some embodiments, the method (e.g., the detecting step) comprises contacting the nucleic acid amplification product with a signal generating oligonucleotide that comprises i) a polynucleotide complementary to a sequence in the amplification product, and ii) a fluorophore and a quencher. In some embodiments, one or more of the at least one oligonucleotide comprise a polynucleotide not complementary to a sequence in the sample nucleic acid that hybridizes to a signal generating oligonucleotide, and wherein the method further comprises contacting the amplification product with the signal generating oligonucleotide that comprises a fluorophore and a quencher. In some embodiments, the method is performed in a single reaction volume. In some embodiments, the method is performed in a single reaction vessel. In some embodiments, the method comprises multiplex amplification.

In some embodiments, the enzymatic activity consists of i) hyperthermophile polymerase activity, and ii) reverse transcriptase activity. In some embodiments, the first oligonucleotide comprises a first polynucleotide complementary to a target sequence in the first strand of the sample nucleic acid, and the second oligonucleotide comprises a second polynucleotide complementary to a target sequence in the second strand of the sample nucleic acid. In some embodiments, the first oligonucleotide comprises a first polynucleotide continuously complementary to a target sequence in the first strand of the sample nucleic acid, and the second oligonucleotide comprises a second polynucleotide continuously complementary to a target sequence in the second strand of the sample nucleic acid. In some embodiments, the first oligonucleotide consists of a first polynucleotide continuously complementary to a target sequence in the first strand of the sample nucleic acid, and the second oligonucleotide consists of a second polynucleotide continuously complementary to a target sequence in the second strand of the sample nucleic acid.

In some embodiments, the first oligonucleotide consists essentially of a first polynucleotide continuously complementary to a sequence in the first strand, and the second oligonucleotide consists essentially of a second polynucleotide continuously complementary to a sequence in the second strand; and/or the nucleic acid amplification product consists essentially of 1) a first nucleotide sequence that is continuously complementary to or substantially identical to the first polynucleotide of the first oligonucleotide, 2) a second nucleotide sequence that is continuously complementary to or substantially identical to the second polynucleotide of the second oligonucleotide, and 3) a spacer sequence comprising 1 to 10 bases.

In some embodiments, the first oligonucleotide consists of a first polynucleotide continuously complementary to a sequence in the first strand, and the second oligonucleotide consists of a second polynucleotide continuously complementary to a sequence in the second strand; and/or the nucleic acid amplification product consists of 1) a first nucleotide sequence that is continuously complementary to or substantially identical to the first polynucleotide of the first oligonucleotide, 2) a second nucleotide sequence that is continuously complementary to or substantially identical to the second polynucleotide of the second oligonucleotide, and 3) a spacer sequence comprising 1 to 10 bases.

In some embodiments, the amplifying comprises contacting sample nucleic acid under helicase-free and recombinase-free isothermal amplification conditions. In some embodiments, the at least one component providing a hyperthermophile polymerase activity comprises a hyperthermophile polymerase or functional fragment thereof, or a polymerase comprising an amino acid sequence that is at least about 90% identical to a hyperthermophile polymerase or functional fragment thereof. In some embodiments, the at least one component providing a hyperthermophile polymerase activity consists of a hyperthermophile polymerase or functional fragment thereof, or a polymerase comprising an amino acid sequence that is at least about 90% identical to a hyperthermophile polymerase or functional fragment thereof. In some embodiments, part (a) (ii) further comprises at least one component providing a reverse transcriptase activity. In some embodiments, the at least one component providing hyperthermophile polymerase activity further provides a reverse transcriptase activity.

In some embodiments, the first oligonucleotide consists essentially of a first polynucleotide continuously complementary to a sequence in a first strand of the target sequence, and the second oligonucleotide consists essentially of a second polynucleotide continuously complementary to a sequence in a second strand of the target sequence. In some embodiments, the first oligonucleotide consists of a first polynucleotide continuously complementary to a sequence in a first strand of the target sequence, and the second oligonucleotide consists of a second polynucleotide continuously complementary to a sequence in a second strand of the target sequence.

In some embodiments, the sample nucleic acid is amplified under helicase-free and recombinase-free isothermal amplification conditions. In some embodiments, the real-time detection activity is provided by a molecular beacon. In some embodiments, kit further comprises instructions for carrying out a method provided herein for determining the presence, absence or amount of a target sequence in sample nucleic acid.

Some embodiments of the methods and compositions provided herein do not comprise agents and/or conditions that denature nucleic acids (e.g., promote strand separation and/or promote unwinding) other than acids and/or low pH conditions. Compositions, kits, and methods for nucleic acid detection wherein nucleic acid strands are dissociated under low pH conditions (e.g., via contact with an acidic lysis buffer) to facilitate subsequent rapid amplification and detection are described in the U.S. Provisional Patent Application No. 63/307,085 entitled “METHOD FOR SEPARATING GENOMIC DNA FOR AMPLIFICATION OF SHORT NUCLEIC ACID TARGETS”, filed on Feb. 5, 2022, the content of which is incorporated herein by reference in its entirety.

### Nucleic Acid, Subjects, Samples and Nucleic Acid Processing

Provided herein are methods and compositions for amplifying nucleic acid. The terms “nucleic acid” and “nucleic acid molecule” may be used interchangeably herein. The terms refer to nucleic acids of any composition, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid can be, or can be from, a plasmid, phage, autonomously replicating sequence (ARS), centromere, artificial chromosome, chromosome, or other nucleic acid able to replicate or be replicated in vitro or in a host cell, a cell, a cell nucleus, a mitochondria, or cytoplasm of a cell. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid may be used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame, “forward” strand or “reverse” strand) and double-stranded polynucleotides. The term “gene” means the segment of DNA involved in producing a polypeptide chain; and generally includes regions preceding and following the coding region (leader and trailer) involved in the transcription/translation of the gene product and the regulation of the transcription/translation, as well as intervening sequences (introns) between individual coding segments (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

In some embodiments of the methods provided herein, one or more nucleic acid targets are amplified. Target nucleic acids may be referred to as target sequences, target polynucleotides, and/or target polynucleotide sequences, and may include double-stranded and single-stranded nucleic acid molecules. Target nucleic acid may be, for example, DNA or RNA. Where the target nucleic acid is an RNA molecule, the molecule may be, for example, double-stranded, single-stranded, or the RNA molecule may comprise a target sequence that is single-stranded. Where the target nucleic acid is double stranded, the target nucleic acid generally includes a first strand and a second strand. A first strand and a second strand may be referred to as a forward strand and a reverse strand and generally are complementary to each other. Where the target nucleic acid is single stranded, a complementary strand may be generated, for example by polymerization and/or reverse transcription, rendering the target nucleic acid double stranded and having a first/forward strand and a second/reverse strand.

A target sequence can refer to either the sense or antisense strand of a nucleic acid sequence, and also can refer to sequences as they exist on target nucleic acids, amplified copies, or amplification products, of the original target sequence. A target sequence can be a subsequence within a larger polynucleotide. The larger polynucleotide can be at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, 100000-fold, 1000000-fold, 1000000-fold, 10000000-fold, 100000000-fold, or a number or a range between any of these values) greater in length than the target sequence. For example, a target sequence can be a short sequence (e.g., about 30 bases) within a nucleic acid fragment, a chromosome, a plasmid, that is targeted for amplification. In some embodiments, a target sequence can refer to a sequence in a target nucleic acid that is complementary to an oligonucleotide (e.g., primer) used for amplifying a nucleic acid. Thus, a target sequence can refer to the entire sequence targeted for amplification or can refer to a subsequence in the target nucleic acid where an oligonucleotide binds. An amplification product can be a larger molecule that comprises the target sequence, as well as at least one other sequence, or other nucleotides. In some embodiments, an amplification product is about the same length as the target sequence, or is exactly the same length as the target sequence. In some embodiments, an amplification product comprises the target sequence. In some embodiments, an amplification product consists of the target sequence.

Target nucleic acid can include, for example, genomic nucleic acid, plasmid nucleic acid, mitochondrial nucleic acid, cellular nucleic acid, extracellular nucleic acid, bacterial nucleic acid and viral nucleic acid. In some embodiments, target nucleic acid can include genomic DNA, chromosomal DNA, plasmid DNA, mitochondrial DNA, a gene, any type of cellular RNA, messenger RNA, bacterial RNA, viral RNA or a synthetic oligonucleotide. A target sequence can, for example, comprise one or more repetitive elements (e.g., multiple repeat sequences, inverted repeat sequences, palindromic sequences, tandem repeats, microsatellites, minisatellites, and the like). Target nucleic acids can include microRNAs, miRNAs, short interfering RNAs (siRNAs), and small temporal RNAs (stRNAs). In some embodiments, genomic target nucleic acid can be within a particular genomic locus or a plurality of genomic loci. A genomic locus can include any or a combination of open reading frame DNA, non-transcribed DNA, intronic sequences, extronic sequences, promoter sequences, enhancer sequences, flanking sequences, or any sequences considered associated with a given genomic locus.

Nucleic acids utilized in methods described herein can be obtained from any suitable biological specimen or sample, and often is isolated from a sample obtained from a subject. A subject can be any living or non-living organism, including but not limited to a human, a non-human animal, a plant, a bacterium, a fungus, a virus, or a protist. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject can be a male or female, and can be any age (e.g., an embryo, a fetus, infant, child, adult).

The term “sample” as used herein shall be given its ordinary meaning, and shall also include both biological and environmental samples that include nucleic acids. The environmental sample can be obtained from a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof. A sample can be any specimen that is isolated or obtained from a subject or part thereof (or a culture thereof). Non-limiting examples of specimens include fluid or tissue from a subject, including, without limitation, blood or a blood product (e.g., serum, plasma, or the like), umbilical cord blood, bone marrow, chorionic villi, amniotic fluid, cerebrospinal fluid, spinal fluid, lavage fluid (e.g., bronchoalveolar, gastric, peritoneal, ductal, ear, arthroscopic), biopsy sample, celocentesis sample, cells (e.g., blood cells) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, hard tissues (e.g., liver, spleen, kidney, lung, or ovary), the like or combinations thereof. The term blood encompasses whole blood, blood product or any fraction of blood, such as serum, plasma, buffy coat, or the like as conventionally defined. The term sample also includes samples that have been manipulated in any way after their procurement, such as by treatment with reagents, culturing, wash(es), and/or enrichment for certain cell populations (such as cancer cells). A sample can include any sample that includes RNA and/or DNA (e.g., in order to determine whether a target DNA and/or target RNA is present among a population of RNAs and/or DNAs). The sample can be derived from any source (e.g., the sample can be a synthetic combination of purified DNAs and/or RNAs). In some embodiments, the sample is a cell-free liquid sample. In some embodiments, the sample is a liquid sample that can comprise cells. The sample can be from a patient (e.g., for the purpose of diagnosis). The sample can be from permeabilized cells, crosslinked cells, and/or tissue section(s). The sample can be from tissues prepared by crosslinking followed by de-lipidation and adjustment to make a uniform refractive index.

A sample can include samples containing spores, viruses, cells, nucleic acids from prokaryotes or eukaryotes, and/or any free nucleic acid. For example, a method described herein can be used for detecting nucleic acid on the outside of spores (e.g., without the need for lysis). A sample can be isolated from any material suspected of containing a target sequence, such as from a subject described above. In some embodiments, a target sequence is present in air, plant, soil, or other materials suspected of containing biological organisms.

Nucleic acids can be derived (e.g., isolated, extracted, purified) from one or more samples by methods known in the art. Any suitable method can be used for isolating, extracting and/or purifying nucleic acid from a biological sample. Nucleic acid can be provided for conducting methods described herein without processing of the sample(s) containing the nucleic acid. For example, in some embodiments, nucleic acid is provided for conducting amplification methods described herein without prior nucleic acid purification. In some embodiments, a target sequence is amplified directly from a sample (e.g., without performing any nucleic acid extraction, isolation, purification and/or partial purification steps). In some embodiments, nucleic acid is provided for conducting methods described herein after processing of the sample(s) containing the nucleic acid. For example, a nucleic acid can be extracted, isolated, purified, or partially purified from the sample(s). The term “isolated” generally refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” can refer to a nucleic acid removed from a subject (e.g., a human subject). An isolated nucleic acid can be provided with fewer non-nucleic acid components (e.g., protein, lipid, carbohydrate) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” generally refers to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid can be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components.

A method of the present disclosure for detecting a target nucleic acid sequence (e.g., gDNA, dsDNA, dsRNA, and the like) in a sample can detect a target nucleic acid sequence (e.g., DNA or RNA) with a high degree of sensitivity. In some embodiments, a method of the present disclosure can be used to detect a target DNA/RNA present in a sample comprising a plurality of RNAs/DNAs (including the target RNA/DNA and a plurality of non-target RNAs/DNAs), wherein the target RNA/DNA is present at one or more copies per 10, 20, 25, 50, 100, 500, 10³, 5×10³, 10⁴, 5×10⁴, 10⁵, 5×10⁵, 10⁶, or 10⁷, non-target DNAs/RNAs. As used herein, the terms “RNA/DNA” and “RNAs/DNAs” shall be given their ordinary meaning, and shall also refer to DNA, or RNA, or a combination of DNA and RNA.

In some embodiments, the threshold of detection, for a subject method of detecting a target RNA/DNA in a sample, is 10 nM or less. The terms “threshold of detection” or “limit of detection” shall be given their ordinary meanings, and shall also describe the minimal amount of target RNA/DNA that must be present in a sample in order for detection to occur. Thus, as an illustrative example, when a threshold of detection is 10 nM, then a signal can be detected when a target RNA/DNA is present in the sample at a concentration of 10 nM or more. In some embodiments, the method has a threshold of detection of no more than, or no less than, 5 nM, 1 nM s, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 500 aM (attomolar), 250 aM, 100 aM, 50 aM, 10 aM, or 1 aM. In some embodiments, a disclosed composition or method exhibits an attamolar (aM), femtomolar (fM), picomolar (pM), and/or nanomolar (nM), sensitivity of detection.

A disclosed sample includes sample nucleic acids (e.g., a plurality of sample nucleic acids). The term “plurality” is used herein to mean two or more. Thus, in some embodiments, a sample includes two or more (e.g., 3, 5, 10, 20, 50, 100, 500, 1000, 5000, or more) sample nucleic acids (e.g., DNAs/RNAs). A disclosed method can be used as a very sensitive way to detect a target nucleic acid present in a sample (e.g., in a complex mixture of nucleic acids such as DNAs/RNAs). In some embodiments the sample includes 5, 10, 20, 25, 50, 100, 500, 10³, 5×10³, 10⁴, 5×10⁴, 10⁵, 5×10⁵, 10⁶, or 10⁷, 50, or more, DNAs/RNAs that differ from one another in sequence. A “sample” can include a target nucleic acid (e.g., target DNA/RNA) and a plurality of non-target DNAs/RNAs. In some embodiments, the target DNA/RNA is present in the sample at one copy per 10, 20, 25, 50, 100, 500, 10³, 5×10³, 10⁴, 5×10⁴, 10⁵, 5×10⁵, 10⁶, or 10⁷, non-target DNAs/RNAs.

The source of the sample can be a (or is suspected of being a) diseased cell, fluid, tissue, or organ; or a normal (non-diseased) cell, fluid, tissue, or organ. In some embodiments, the source of the sample is a (or is suspected of being a) pathogen-infected cell, tissue, or organ. For example, the source of a sample can be an individual who may or may not be infected- and the sample can be any biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual. The sample can be a cell-free liquid sample or a liquid sample that comprise cells. Pathogens can be viruses, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include, e.g., immunodeficiency virus (e.g., HIV); influenza virus; dengue; West Nile virus; herpes virus; yellow fever virus; Hepatitis C virus; Hepatitis A virus; Hepatitis B virus; papillomavirus; and the like. Pathogenic viruses can include DNA viruses such as: a papovavirus (e.g., HPV, polyomavirus); a hepadnavirus; a herpesvirus (e.g., HSV (e.g., HSV I, HSV II), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. Non-limiting examples of pathogens include Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, human serum parvo-like virus, respiratory syncytial virus, measles virus, adenovirus, human T-cell leukemia viruses, murine leukemia virus, mumps virus, vesicular stomatitis virus, Sindbis virus, lymphocytic choriomeningitis virus, wart virus, blue tongue virus, Sendai virus, feline leukemia virus, reovirus, polio virus, simian virus 40, mouse mammary tumor virus, dengue virus, rubella virus, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae.

### Amplification

Provided herein are methods for amplifying nucleic acid. In some embodiments, nucleic acids are amplified using a suitable amplification process. Nucleic acid amplification typically involves enzymatic synthesis of nucleic acid amplicons (copies), which contain a sequence complementary to a nucleotide sequence being amplified. In some embodiments, an amplification method is performed in a single vessel, a single chamber, and/or a single volume (i.e., contiguous volume). In some embodiments, an amplification method and a detection method (e.g., a detection method described herein) are performed in a single vessel, a single chamber, and/or a single volume (i.e., contiguous volume).

The terms “amplify”, “amplification”, “amplification reaction”, or “amplifying” refer to any in vitro process for multiplying the copies of a target nucleic acid. Amplification sometimes refers to an “exponential” increase in target nucleic acid. “Amplifying” can also refer to linear increases in the numbers of a target nucleic acid, but is different than a one-time, single primer extension step. In some embodiments a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may limit inaccuracies associated with depleted reactants in certain amplification reactions, and also may reduce amplification biases due to nucleotide sequence or species abundance of the target. In some embodiments a one-time primer extension may be performed as a prelude to linear or exponential amplification.

A generalized description of an amplification process is presented herein. Primers (e.g., oligonucleotides described herein) and target nucleic acid are contacted, and complementary sequences anneal or hybridize to one another, for example. Primers can anneal to a target nucleic acid, at or near (e.g., adjacent to, abutting, and the like) a sequence of interest. A primer annealed to a target may be referred to as a primer-target hybrid, hybridized primer-target, or a primer-target duplex. The terms near or adjacent to when referring to a nucleotide sequence of interest refer to a distance (e.g., number of bases) or region between the end of the primer and the nucleotide or nucleotides (e.g., nucleotide sequence) of a target. Generally, adjacent is in the range of about 1 nucleotide to about 50 nucleotides (e.g., about 1, 2, 3, 4, 5, 6, 7, 8, 9, 10, 20, 30, 40, or 50 nucleotide(s)) away from a nucleotide or nucleotide sequence of interest. In some embodiments, primers in a set (e.g., a pair of primers, a forward and a reverse primer, a first oligonucleotide and a second oligonucleotide) anneal within about 1 to 20 nucleotides from a nucleotide or nucleotide sequence of interest and produce amplified products. In some embodiments, primers anneal within a nucleotide or a nucleotide sequence of interest. After annealing, each primer is extended along the target (i.e., template strand) by a polymerase to generate a complementary strand. Several cycles of primer annealing and extension can be carried out, for example, until a detectable amount of amplification product is generated. In some embodiments, where a target nucleic acid is RNA, a DNA copy (cDNA) of the target RNA is synthesized prior to or during the amplification step by reverse transcription.

Components of an amplification reaction (e.g., the one or more amplification reagents) can include, for example, one or more primers (e.g., individual primers, primer pairs, primer sets, oligonucleotides, multiple primer sets for multiplex amplification, and the like), nucleic acid target(s) (e.g., target nucleic acid from a sample), one or more polymerases, nucleotides (e.g., dNTPs and the like), and a suitable buffer (e.g., a buffer comprising a detergent, a reducing agent, monovalent ions, and divalent ions). An amplification reaction can further include one or more of: a reverse transcriptase, a reverse transcription primer, and one or more detection agents.

Nucleic acid amplification can be conducted in the presence of native nucleotides, for example, dideoxyribonucleoside triphosphates (dNTPs), and/or derivatized nucleotides. A native nucleotide generally refers to adenylic acid, guanylic acid, cytidylic acid, thymidylic acid, or uridylic acid. A derivatized nucleotide generally is a nucleotide other than a native nucleotide. A ribonucleoside triphosphate is referred to as NTP or rNTP, where N can be A, G, C, U. A deoxynucleoside triphosphate substrates is referred to as dNTP, where N can be A, G, C, T, or U. Monomeric nucleotide subunits may be denoted as A, G, C, T, or U herein with no particular reference to DNA or RNA. In some embodiments, non-naturally occurring nucleotides or nucleotide analogs, such as analogs containing a detectable label (e.g., fluorescent or colorimetric label), may be used. For example, nucleic acid amplification can be carried out in the presence of labeled dNTPs, for example, radiolabels such as 32p, 33p, 1251, or 35S; enzyme labels such as alkaline phosphatase; fluorescent labels such as fluorescein isothiocyanate (FITC); or other labels such as biotin, avidin, digoxigenin, antigens, haptens, or fluorochromes. In some embodiments, nucleic acid amplification may be carried out in the presence of modified dNTPs, for example, heat activated dNTPs (e.g., CleanAmp™ dNTPs from TriLink).

The one or more amplification reagents can include non-enzymatic components and enzymatic components. Non-enzymatic components can include, for example, primers, nucleotides, buffers, salts, reducing agents, detergents, and ions. In some embodiments, the Non-enzymatic components do not include proteins (e.g., nucleic acid binding proteins), enzymes, or proteins having enzymatic activity, for example, polymerases, reverse transcriptases, helicases, topoisomerases, ligases, exonucleases, endonucleases, restriction enzymes, nicking enzymes, recombinases and the like. In some embodiments, an enzymatic component consists of a polymerase or consists of a polymerase and a reverse transcriptase. Accordingly, such enzymatic components would exclude other proteins (e.g., nucleic acid binding proteins and/or proteins having enzymatic activity), for example, helicases, topoisomerases, ligases, exonucleases, endonucleases, restriction enzymes, nicking enzymes, recombinases, and the like.

In some embodiments, amplification conditions comprise an enzymatic activity (e.g., an enzymatic activity provided by a polymerase or provided by a polymerase and a reverse transcriptase). In some embodiments, the enzymatic activity does not include enzymatic activity provided by enzymes other than the polymerase and/or the reverse transcriptase, for example, helicases, topoisomerases, ligases, exonucleases, endonucleases, restriction enzymes, nicking enzymes, recombinases, and the like. A polymerase activity and a reverse transcriptase activity can be provided by separate enzymes or separate enzyme types (e.g., polymerase(s) and reverse transcriptase(s)), or provided by a single enzyme or enzyme type (e.g., polymerase(s)).

Amplification of nucleic acid can comprise a non-thermocycling type of PCR. In some embodiments, amplification of nucleic acid comprises an isothermal amplification process, for example an isothermal polymerase chain reaction (iPCR). Isothermal amplification generally is an amplification process performed at a constant temperature. Terms such as isothermal conditions, isothermally and constant temperature generally refer to reaction conditions where the temperature of the reaction is kept essentially constant during the course of the amplification reaction. Isothermal amplification conditions generally do not include a thermocycling (i.e., cycling between an upper temperature and a lower temperature) component in the amplification process. When amplifying under isothermal conditions, the reaction can be kept at an essentially constant temperature, which means the temperature may not be maintained at precisely one temperature. For example, small fluctuations in temperature (e.g., ±1 to 5° C.) may occur in an isothermal amplification process due to, for example, environmental or equipment-based variables. Often, the entire reaction volume is kept at an essentially constant temperature, and isothermal reactions herein generally do not include amplification conditions that rely on a temperature gradient generated within a reaction vessel and/or convective-flow based temperature cycling.

Isothermal amplification reactions herein can be conducted at an essentially constant temperature. In some embodiments, isothermal amplification reactions herein are conducted at a temperature of about 55° C. to a temperature of about 75° C., for example at a temperature of, or a temperature of about, 55, 56, 57, 58, 59, 60, 61, 62, 63, 64, 65, 66, 67, 68, 69, 70, 71, 72, 73, 74, or about 75° C., or a number or a range between any two of these values. In some embodiments, a temperature element (e.g., heat source) is kept at an essentially constant temperature, for example an essentially constant temperature at or below about 75° C., at or below about 70 degrees Celsius, at or below about 65° C., or at or below about 60° C.

An amplification process herein can be conducted over a certain length of time, for example until a detectable nucleic acid amplification product is generated. A nucleic acid amplification product may be detected by any suitable detection process and/or a detection process described herein. The amplification process can be conducted over a length of time within about 20 minutes or less, or about 10 minutes or less. For example, an amplification process can be conducted within about 1, 2, 3, 4, 5, 6, 7, 8, 9, 10, 11, 12, 13, 14, 15, 16, 17, 18, 19 or 20 minutes, or a number or a range between any two of these values.

Nucleic acid targets can be amplified without exposure to agents or conditions that denature nucleic acid, in some embodiments. Nucleic acid targets can be amplified without exposure to agents or conditions that promote strand separation during the amplification step (and/or other steps) in some embodiments. Nucleic acid targets can be amplified without exposure to agents or conditions that promote unwinding during the amplification step (and/or other steps) in some embodiments. Agents or conditions that denature nucleic acid and/or promote strand separation and/or promote unwinding may include, for example, thermal conditions (e.g., high temperatures), pH conditions (e.g., high or low pH), chemical agents, proteins (e.g., enzymatic agents), and the like.

In some embodiments, the methods disclosed herein does not comprise thermal denaturation (e.g., heating a solution containing a nucleic acid to an elevated temperature, such as, for example a temperature above 75° C., 80° C., 90° C., or 95° C., or higher) or protein-based (e.g., enzymatic) denaturation of a nucleic acid. Protein-based (e.g., enzymatic) denaturation can comprise contacting a nucleic acid with one or more of a helicase, a topoisomerase, a ligase, an exonuclease, an endonuclease, a restriction enzyme, a nicking enzyme, a recombinase, a RNA replicase, and a nucleic acid binding protein (e.g., single-stranded binding protein). In some embodiments, the compositions provided herein do not comprise a helicase, a topoisomerase, a ligase, an exonuclease, an endonuclease, a restriction enzyme, a nicking enzyme, a recombinase, a RNA replicase, and/or a nucleic acid binding protein (e.g., single-stranded binding protein). In some embodiments, the compositions and methods provided herein do not comprise intercalators, alkylating agents, and/or chemicals such as formamide, glycerol, urea, dimethyl sulfoxide (DMSO), or N,N,N-trimethylglycine (betaine). In some embodiments, the disclosed methods do not comprise contacting a nucleic acid with denaturing agents (e.g., formamide). In some embodiments, the amplifying step does not comprise agents and/or conditions that denature nucleic acids (e.g., promote strand separation and/or promote unwinding). In some embodiments, the amplifying step (e.g., step (c)) does not comprise agents and/or conditions that denature nucleic acids (e.g., promote strand separation and/or promote unwinding) other than a polymerase (e.g., a hyperthermophile polymerase). In some embodiments, the methods and compositions provided herein not comprise agents and/or conditions that denature nucleic acids (e.g., promote strand separation and/or promote unwinding) other than a polymerase (e.g., a hyperthermophile polymerase) and/or low pH conditions (e.g., contact with acid(s)).

Nucleic acid targets can be amplified without exposure to agents or conditions that promote strand separation and/or unwinding, for example a helicase, a topoisomerase, a ligase, an exonuclease, an endonuclease, a restriction enzyme, a nicking enzyme, a recombinase, a RNA replicase, a nucleic acid binding protein (e.g., single-stranded binding protein), or any combination thereof. For example, nucleic acid targets can be amplified without exposure to a helicase, including but not limited to DNA helicases and RNA helicases. Amplification conditions that do not include use of a helicase are helicase-free amplification conditions.

Nucleic acid targets can be amplified without exposure to a recombinase, including but not limited to, Cre recombinase, Hin recombinase, Tre recombinase, FLP recombinase, RecA, RAD51, RadA, T4 uvsX. In some embodiments, nucleic acid targets are amplified without exposure to a recombinase accessory protein, for example, a recombinase loading factor (e.g., T4 uvsY). Nucleic acid targets can be amplified without exposure to a nucleic acid binding protein (e.g., single-stranded binding protein or single-strand DNA-binding protein (SSB)), for example, T4 gp32. In some embodiments, nucleic acid targets are amplified without exposure to a topoisomerase. Nucleic acid targets can be amplified with or without exposure to agents or conditions that destabilize nucleic acid. As used herein, the term “destabilization” shall be given its ordinary meaning, and shall also refer to a disruption in the overall organization and geometric orientation of a nucleic acid molecule (e.g., double helical structure) by one or more of tilt, roll, twist, slip, and flip effects (e.g., as described in Lenglet et al., (2010) Journal of Nucleic Acids Volume 2010, Article ID 290935, 17 pages). Destabilization generally does not refer to melting or separation of nucleic acid strands (e.g., denaturation). Nucleic acid destabilization can be achieved, for example, by exposure to agents such as intercalators or alkylating agents, and/or chemicals such as formamide, urea, dimethyl sulfoxide (DMSO), or N,N,N-trimethylglycine (betaine). In some embodiments, methods provided herein include use of one or more destabilizing agents. In some embodiments, methods provided herein exclude use of destabilizing agents. In some embodiments, nucleic acid targets are amplified without exposure to a ligase and/or an RNA replicase.

Nucleic acid targets can be amplified without cleavage or digestion, in some embodiments. For example, nucleic acid targets can be amplified without prior exposure to one or more cleavage agents, and intact nucleic acid is amplified. In some embodiments, nucleic acid targets are amplified without exposure to one or more cleavage agents during amplification. In some embodiments, nucleic acid targets are amplified without exposure to one or more cleavage agents after amplification. Amplification conditions that do not include use of a cleavage agent may be referred to herein as cleavage agent-free amplification conditions. The term “cleavage agent” generally refers to an agent, sometimes a chemical or an enzyme that can cleave a nucleic acid at one or more specific or non-specific sites. Specific cleavage agents often cleave specifically according to a particular nucleotide sequence at a particular site. Cleavage agents can include endonucleases (e.g., restriction enzymes, nicking enzymes, and the like); exonucleases (DNAses, RNAses (e.g., RNAseH), 5′ to 3′ exonucleases (e.g. exonuclease II), 3′ to 5′ exonucleases (e.g. exonuclease I), and poly(A)-specific 3′ to 5′ exonucleases); and chemical cleavage agents.

Nucleic acid targets can be amplified without use of restriction enzymes and/or nicking enzymes. In some embodiments, nucleic acid is amplified without prior exposure to restriction enzymes and/or nicking enzymes. In some embodiments, nucleic acid is amplified without exposure to restriction enzymes and/or nicking enzymes during amplification. In some embodiments, nucleic acid is amplified without exposure to restriction enzymes and/or nicking enzymes after amplification. Nucleic acid targets can be amplified without exonuclease treatment. Exonucleases include, for example, DNAses, RNAses (e.g., RNAseH), 5′ to 3′ exonucleases (e.g. exonuclease II), 3′ to 5′ exonucleases (e.g. exonuclease I), and poly(A)-specific 3′ to 5′ exonucleases. In some embodiments, nucleic acid is amplified without exonuclease treatment prior to, during, and/or after amplification. Amplification conditions that do not include use of an exonuclease are exonuclease-free amplification conditions. In some embodiments, nucleic acid is amplified without DNAse treatment and/or RNAse treatment. In some embodiments, nucleic acid is amplified without RNAseH treatment.

An amplified nucleic acid may be referred to herein as a nucleic acid amplification product or amplicon. In some embodiments, the amplification product includes naturally occurring nucleotides, non-naturally occurring nucleotides, nucleotide analogs and the like and combinations of the foregoing. An amplification product typically has a nucleotide sequence that is identical to or substantially identical to a sequence in a sample nucleic acid (e.g., target sequence) or complement thereof. A “substantially identical” nucleotide sequence in an amplification product will generally have a high degree of sequence identity to the nucleotide sequence being amplified or complement thereof (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% sequence identity), and variations sometimes are a result of polymerase infidelity or other variables.

In some embodiments, a nucleic acid amplification product comprises a polynucleotide that is continuously complementary to or substantially identical to a target sequence in sample nucleic acid. Continuously complementary generally refers to a nucleotide sequence in a first strand, for example, where each base in order (e.g., read 5′ to 3′) pairs with a correspondingly ordered base in a second strand, and there are no gaps, additional sequences or unpaired bases within the sequence considered as continuously complementary. Stated another way, continuously complementary generally refers to all contiguous bases of a nucleotide sequence in a first stand being complementary to corresponding contiguous bases of a nucleotide sequence in a second strand. For example, a first strand having a sequence 5′-ATGCATGCATGC-3′ (SEQ ID NO: 3) would be considered as continuously complementary to a second strand having a sequence 5′-GCATGCATGCAT-3′ (SEQ ID NO: 4), where all contiguous bases in the first strand are complementary to all corresponding contiguous bases in the second strand. However, a first strand having a sequence 5′-ATGCATAAAAAAGCATGC-3′ (SEQ ID NO: 5) would not be considered as continuously complementary to a second strand having a sequence 5′-GCATGCATGCAT-3′ (SEQ ID NO: 4), because the sequence of six adenines (6 As) in the middle of the first strand would not pair with bases in the second strand. A continuously complementary sequence sometimes is about 5 to about 25 contiguous bases in length, e.g., about 5, 6, 7, 8, 9, 10, 11, 12, 13, 14, 15, 16, 17, 18, 19, 20, 21, 22, 23, 24, 25, or a range between any two of these values, contiguous bases in length. In some embodiments, a nucleic acid amplification product consists of a polynucleotide that is continuously complementary to or substantially identical to a target sequence in sample nucleic acid. Accordingly, in some embodiments, a nucleic acid amplification product does not include any additional sequences (e.g., at the 5′ and/or 3′ end, or within the product) that are not continuously complementary to or substantially identical to a target sequence, for example, additional sequences incorporated into an amplification product by way of tailed primers or ligation, and/or additional sequences providing cleavage agent recognition sites (e.g., nicking enzyme recognition sites). Generally, unless a target sequence comprises tandem repeats, an amplification product does not include product in the form of tandem repeats.

Nucleic acid amplification products can comprise sequences complementary to or substantially identical to one or more primers used in an amplification reaction. In some embodiments, a nucleic acid amplification product comprises a first nucleotide sequence that is continuously complementary to or identical to a first primer sequence, and a second nucleotide sequence that is continuously complementary to or identical to a second primer sequence.

Nucleic acid amplification products can comprise a spacer sequence. As described herein, a spacer sequence in an amplification product is a sequence (1 or more bases) continuously complementary to or substantially identical to a portion of a target sequence in the sample nucleic acid, and is flanked by sequences in the amplification product that are complementary to or substantially identical to one or more primers used in an amplification reaction. A spacer sequence flanked by sequences in the amplification product generally lies between a first sequence (complementary to or substantially identical to a first primer) and a second sequence (complementary to or substantially identical to a second primer). Thus, an amplification product typically includes a first sequence followed by a spacer sequences followed by a second sequence. A spacer sequence generally is not complementary to or substantially identical to a sequence in the primer(s). A spacer sequence can be, or can comprise, about 1 to 10 bases, including 1, 2, 3, 4, 5, 6, 7, 8, 9, or 10 bases. In some embodiments, a nucleic acid amplification product consists of, or consists essentially of, a first nucleotide sequence that is continuously complementary to or identical to a first primer sequence, a second nucleotide sequence that is continuously complementary to or identical to a second primer sequence, and a spacer sequence. In some embodiments, a nucleic acid amplification product does not include any additional sequences (e.g., at the 5′ and/or 3′ end; or within the product) that are not continuously complementary to or identical to a first primer sequence and a second primer sequence, and are not part of a spacer sequence, for example, additional sequences incorporated into an amplification product by way of tailed or looped primers, ligation or other mechanism. In some embodiments, a nucleic acid amplification product generally does not include additional sequences (e.g., at the 5′ and/or 3′ end; or within the product) that are not continuously complementary to or identical to a first primer sequence and a second primer sequence, and are not part of a spacer sequence, for example, additional sequences incorporated into an amplification product by way of tailed or looped primers, ligation or other mechanism. However, in such embodiments, a nucleic acid amplification product may include, for example, some mismatched (i.e., non-complementary) bases or one more extra bases (e.g., at the 5′ and/or 3′ end; or within the product) introduced into the product by way of error or promiscuity in the amplification process.

Nucleic acid amplification products can be up to 50 bases in length, including 10, 15, 20, 25, 30, 35, 40, 45, 50, or a number or a range between any two of these values, bases long. In some embodiments, nucleic acid amplification products for a given target sequence have the same length or substantially the same length (e.g., within 1 to 10 bases). Accordingly, nucleic acid amplification products for a given target sequence may produce a single signal (e.g., band on an electrophoresis gel) and generally do not produce multiple signals indicative of multiple lengths (e.g., a ladder or smear on an electrophoresis gel). For multiplex reactions, nucleic acid amplification products for different target sequences may have different lengths.

The methods and components described herein can be used for multiplex amplification which generally refers to the amplification of more than one nucleic acid of interest (e.g., amplification or more than one target sequence). For example, multiplex amplification can refer to amplification of multiple sequences from the same sample or amplification of one of several sequences in a sample. Multiplex amplification also can refer to amplification of one or more sequences present in multiple samples either simultaneously or in step-wise fashion. For example, a multiplex amplification can be used for amplifying least two target sequences that are capable of being amplified (e.g., the amplification reaction comprises the appropriate primers and enzymes to amplify at least two target sequences). In some embodiments, an amplification reaction is prepared to detect at least two target sequences, but only one of the target sequences is present in the sample being tested, such that both sequences are capable of being amplified, but only one sequence is amplified. In some embodiments, where two target sequences are present, an amplification reaction results in the amplification of both target sequences. A multiplex amplification reaction can result in the amplification of one, some, or all of the target sequences for which it comprises the appropriate primers and enzymes. In some embodiments, an amplification reaction is prepared to detect two sequences with one pair of primers, where one sequence is a target sequence and one sequence is a control sequence (e.g., a synthetic sequence capable of being amplified by the same primers as the target sequence and having a different spacer base or sequence than the target). In some embodiments, an amplification reaction is prepared to detect multiple sets of sequences with corresponding primer pairs, where each set includes a target sequence and a control sequence.

### Primers

Nucleic acid amplification generally is conducted in the presence of one or more primers. A primer is generally characterized as an oligonucleotide that includes a nucleotide sequence capable of hybridizing or annealing to a target nucleic acid, at or near (e.g., adjacent to) a specific region of interest (i.e., target sequence). Primers can allow for specific determination of a target nucleic acid nucleotide sequence or detection of the target nucleic acid (e.g., presence or absence of a sequence), or feature thereof, for example. A primer can be naturally occurring or synthetic. The term specific, or specificity, generally refers to the binding or hybridization of one molecule to another molecule, such as a primer for a target polynucleotide. That is, specific or specificity refers to the recognition, contact, and formation of a stable complex between two molecules, as compared to substantially less recognition, contact, or complex formation of either of those two molecules with other molecules. The term anneal or hybridize generally refers to the formation of a stable complex between two molecules. The terms primer, oligo, or oligonucleotide may be used interchangeably herein, when referring to primers.

A primer can be designed and synthesized using suitable processes, and can be of any length suitable for hybridizing to a target sequence and performing an amplification process described herein. Primers often are designed according to a sequence in a target nucleic acid. A primer in some embodiments may be about 5 to about 30 bases in length, for example 5, 6, 7, 8, 9, 10, 11, 12, 13, 14, 15, 16, 17, 18, 19, 20, 21, 22, 23, 24, 25, 26, 27, 28, 29 or 30 bases in length. A primer may be composed of naturally occurring and/or non-naturally occurring nucleotides (e.g., modified nucleotides, labeled nucleotides), or a mixture thereof. Modifications and modified bases may include, for example, phosphorylation, (e.g., 3′ phosphorylation, 5′ phosphorylation); attachment chemistry or linkers modifications (e.g., Acrydite™, adenylation, azide (NHS ester), digoxigenin (NHS ester), cholesteryl-TEG, I-Linker™, amino modifiers (e.g., amino modifier C6, amino modifier C12, amino modifier C6 dT, Uni-Link™ amino modifier), alkynes (e.g., 5′ hexynyl, 5-octadiynyl dU), biotinylation (e.g., biotin, biotin (azide), biotin dT, biotin-TEG, dual biotin, PC biotin, desthiobiotin-TEG), thiol modifications (e.g., thiol modifier C3 S—S, dithiol, thiol modifier C6 S—S)); fluorophores (e.g., Freedom™ Dyes, Alexa Fluor® Dyes, LI-COR IRDyes®, ATTO™ Dyes, Rhodamine Dyes, WellRED Dyes, 6-FAM (azide), Texas Red®-X (NHS ester), Lightcycler® 640 (NHS ester), Dy 750 (NHS ester)); Iowa Black® dark quenchers modifications (e.g., Iowa Black® FQ, Iowa Black® RQ); dark quenchers modifications (e.g., Black Hole Quencher®-1, Black Hole Quencher®-2, Dabcyl); spacers (C3 spacer, PC spacer, hexanediol, spacer 9, spacer 18, 1′,2′-dideoxyribose (dSpacer); modified bases (e.g., 2-aminopurine, 2,6-diaminopurine (2-amino-dA), 5-bromo dU, deoxyUridine, inverted dT, inverted dideoxy-T, dideoxy-C, 5-methyl dC, deoxyInosine, Super T®, Super GR, locked nucleic acids (LNA's), 5-nitroindole, 2′-O-methyl RNA bases, hydroxmethyl dC, UNA unlocked nucleic acid (e.g., UNA-A, UNA-U, UNA-C, UNA-G), Iso-dC, Iso-dG, Fluoro C, Fluoro U, Fluoro A, Fluoro G); phosphorothioate (PS) bonds modifications (e.g., phosphorothioated DNA bases, phosphorothioated RNA bases, phosphorothioated 2′ O-methyl bases, phosphorothioated LNA bases); and click chemistry modifications. In some embodiments, modifications and modified bases include uracil bases, ribonucleotide bases, O-methyl RNA bases, PS linkages, 3′ phosphate groups, spacer bases (such as C3 spacer or other spacer bases). For example, a primer may comprise one or more O-methyl RNA bases (e.g., 2′-O-methyl RNA bases). 2′-O-methyl RNA generally is a post-transcriptional modification of RNA found in tRNA and other small RNAs. Primers can be directly synthesized that include 2′-O-methyl RNA bases. This modification can, for example, increase Tm of RNA:RNA duplexes and provide stability in the presence of single-stranded ribonucleases and DNases. 2′-O-methyl RNA bases may be included in primers, for example, to increase stability and binding affinity to a target sequence. In some embodiments, a primer may comprise one or more phosphorothioate (PS) linkages (e.g., PS bond modifications). A PS bond substitutes a sulfur atom for a non-bridging oxygen in the phosphate backbone of a primer. This modification typically renders the internucleotide linkage resistant to nuclease degradation. PS bonds can be introduced between about the last 3 to 5 nucleotides at the 5′-end or the 3′-end of a primer to inhibit exonuclease degradation, for example. PS bonds included throughout an entire primer can help reduce attack by endonucleases, in some embodiments. A primer can, for example, comprise a 3′ phosphate group. 3′ phosphorylation can inhibit degradation by certain 3′-exonucleases and can be used to block extension by DNA polymerases, in certain instances. In some embodiments, a primer comprises one or more spacer bases (e.g., one or more C3 spacers). A C3 spacer phosphoramidite can be incorporated internally or at the 5′-end of a primer. Multiple C3 spacers can be added at either end of a primer to introduce a long hydrophilic spacer arm for the attachment of fluorophores or other pendent groups, for example.

A primer can comprise DNA bases, RNA bases, or both, where one or more of the DNA bases and RNA bases is modified or unmodified. For example, a primer can be a mixture of DNA bases and RNA bases. The primer can consist of DNA bases (e.g., modified DNA bases and/or unmodified DNA bases). In some embodiments, the primer consists of unmodified DNA bases. In some embodiments, the primer consists of modified DNA bases. The primer can consist of RNA bases (e.g., modified RNA bases and/or unmodified RNA bases). In some embodiments, the primer consists of unmodified RNA bases. In some embodiments, the primer consists of modified RNA bases. In some embodiments, a primer comprises no RNA bases. In some embodiments, a primer comprises no DNA bases. In some embodiments, the primer comprises no cleavage agent recognition sites (e.g., no nicking enzyme recognition sites). In some embodiments, a primer comprises no tail (e.g., no tail comprising a nicking enzyme recognition site).

All or a portion of a primer sequence can be complementary or substantially complementary to a target nucleic acid, in some embodiments. Substantially complementary with respect to sequences generally refers to nucleotide sequences that will hybridize with each other. The stringency of the hybridization conditions can be altered to tolerate varying amounts of sequence mismatch. The target and primer sequences can be, for example, at least 75% complementary to each other, including 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to each other. Primers that are substantially complimentary to a target nucleic acid sequence typically are also substantially identical to the complement of the target nucleic acid sequence (i.e., the sequence of the anti-sense strand of the target nucleic acid). The primer and the anti-sense strand of the target nucleic acid can be at least 75% identical in sequence, for example 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each other.

In some embodiments, primers comprise a pair of primers. A pair of primers may include a forward primer and a reverse primer (e.g., primers that bind to the sense and antisense strands of a target nucleic acid). In some embodiments, primers consist of a pair of primers (i.e. a forward primer and a reverse primer). Accordingly, in some embodiments, amplification of a target sequence is performed using a pair of primers and no additional primers or oligonucleotides are included in the amplification of the target sequence (e.g., the amplification reaction components comprise no additional primer pairs for a given target sequence, no nested primers, no bumper primers, no oligonucleotides other than the primers, no probes, and the like). In some embodiments, primers consist of a pair of primers. In some embodiments, an amplification reaction can include additional primer pairs for amplifying different target sequences, such as in a multiplex amplification. In some embodiments, primers consist of a pair of primers, however, in some embodiments, an amplification reaction can include additional primers, oligonucleotides or probes for a detection process that are not considered part of amplification. In some embodiments, primers are used in sets. An amplification primer set can include a pair of forward and reverse primers for a given target sequence. For multiplex amplification, primers that amplify a first target sequence are considered a primer set, and primers that amplify a second target sequence are considered a different primer set.

Amplification reaction components can comprise, or consist of, a first primer (first oligonucleotide) complementary to a target sequence in a first strand (e.g., sense strand, forward strand) of a sample nucleic acid, and a second primer (second oligonucleotide) complementary to a target sequence in a second strand (e.g., antisense strand, reverse strand) of a sample nucleic acid. In some embodiments, a first primer (first oligonucleotide) comprises a first polynucleotide continuously complementary to a target sequence in a first strand of sample nucleic acid, and a second primer (second oligonucleotide) comprises a second polynucleotide continuously complementary to a target sequence in a second strand of sample nucleic acid. Continuously complementary for a primer-target generally refers to a nucleotide sequence in a primer, where each base in order pairs with a correspondingly ordered base in a target sequence, and there are no gaps, additional sequences or unpaired bases within the sequence considered as continuously complementary. In some embodiments, a primer does not include any additional sequences (e.g., at the 5′ and/or 3′ end, or within the primer) that are not continuously complementary to a target sequence, for example, additional sequences present in tailed primers or looped primers, and/or additional sequences providing cleavage agent recognition sites (e.g., nicking enzyme recognition sites). In some embodiments, amplification reaction components do not comprise primers comprising additional sequences (i.e., sequences other than the sequence that is continuously complementary to a target sequence), for example, tailed primers, looped primers, primers capable of forming step-loop structures, hairpin structures, and/or additional sequences providing cleavage agent recognition sites (e.g., nicking enzyme recognition sites), and the like.

The primer, in some embodiments, can contain a modification such as one or more inosines, abasic sites, locked nucleic acids, minor groove binders, duplex stabilizers (e.g., acridine, spermidine), Tm modifiers or any modifier that changes the binding properties of the primer. The primer, in some embodiments, can contain a detectable molecule or entity (e.g., a fluorophore, radioisotope, colorimetric agent, particle, enzyme and the like).

### Polymerase

Amplification reaction components (e.g., one or more amplification reagents) can comprise one or more polymerases. Polymerases are proteins capable of catalyzing the specific incorporation of nucleotides to extend a 3′ hydroxyl terminus of a primer molecule, for example, an amplification primer described herein, against a nucleic acid target sequence (e.g., to which a primer is annealed). Non-limiting examples of polymerases include thermophilic or hyperthermophilic polymerases that can have activity at an elevated reaction temperature (e.g., above 55, 60, 65, 70, 75, 80, 85, 90, 95, or 100° C.). A hyperthermophilic polymerase may be referred to as a hyperthermophile polymerase. A polymerase may or may not have strand displacement capabilities. In some embodiments, a polymerase can incorporate about 1 to about 50 nucleotides in a single synthesis, for example about 1, 5, 10, 15, 20, 25, 30, 35, 40, 45 or 50 nucleotides, or a number or a range between any two of these values, in a single synthesis.

The amplification reaction components can comprise one or more DNA polymerases selected from: 9°N DNA polymerase; 9°Nm™ DNA polymerase; Therminator™ DNA Polymerase; Therminator™ II DNA Polymerase; Therminator™ III DNA Polymerase; Therminator™ y DNA Polymerase; Bst DNA polymerase; Bst DNA polymerase (large fragment); Phi29 DNA polymerase, DNA polymerase I (E. coli), DNA polymerase I, large (Klenow) fragment; Klenow fragment (3′-5′ exo-); T4 DNA polymerase; T7 DNA polymerase; Deep VentR™ (exo-) DNA Polymerase; Deep VentR™ DNA Polymerase; DyNAzyme™ EXT DNA; DyNAzyme™ II Hot Start DNA Polymerase; Phusion™ High-Fidelity DNA Polymerase; VentR DNA Polymerase; VentR® (exo-) DNA Polymerase; RepliPHI™ Phi29 DNA Polymerase; rBst DNA Polymerase, large fragment (IsoTherm™ DNA Polymerase); MasterAmp™ AmpliTherm™ DNA Polymerase; Tag DNA polymerase; Tth DNA polymerase; Tfl DNA polymerase; Tgo DNA polymerase; SP6 DNA polymerase; Tbr DNA polymerase; DNA polymerase Beta; and ThermoPhi DNA polymerase.

In some embodiments, the amplification reaction components comprise one or more hyperthermophile DNA polymerases (e.g., hyperthermophile DNA polymerases that are thermostable at high temperatures). The hyperthermophile DNA polymerase can have a half-life of about 5 to 10 hours at 95° C. and a half-life of about 1 to 3 hours at 100° C. For example, the amplification reaction components can comprise one or more hyperthermophile DNA polymerases from Archaea (e.g., hyperthermophile DNA polymerases from Thermococcus, or hyperthermophile DNA polymerases from Thermococcaceaen archaean). In some embodiments, amplification reaction components comprise one or more hyperthermophile DNA polymerases from Pyrococcus, Methanococcaceae, Methanococcus, or Thermus. In some embodiments, amplification reaction components comprise one or more hyperthermophile DNA polymerases from Thermus thermophiles.

In some embodiments, amplification reaction components comprise a hyperthermophile DNA polymerase or functional fragment thereof. A functional fragment generally retains one or more functions of a full-length polymerase, for example, the capability to polymerize DNA (e.g., in an amplification reaction). In some instances, a functional fragment performs a function (e.g., polymerization of DNA in an amplification reaction) at a level that is at least about 50%, at least about 75%, at least about 90%, at least about 95% the level of function for a full length polymerase. Levels of polymerase activity can be assessed, for example, using a detectable nucleic acid amplification method, such as a detectable nucleic acid amplification method described herein. In some embodiments, amplification reaction components comprise a hyperthermophile DNA polymerase comprising an amino acid sequence of SEQ ID NO: 1 or SEQ ID NO: 2, or a functional fragment of SEQ ID NO: 1 or SEQ ID NO: 2.

In some embodiments, amplification reaction components (e.g., one or more amplification reagents) comprise a polymerase comprising an amino acid sequence that is at least about 90% identical to a hyperthermophile polymerase or a functional fragment thereof. In some embodiments, amplification reaction components comprise a polymerase comprising an amino acid sequence that is at least about 90%, 95%, or 99% identical to the amino acid sequence of SEQ ID NO: 1 or SEQ ID NO: 2, or a functional fragment thereof.

The polymerase can possess reverse transcription capabilities. In such embodiments, the amplification reaction can amplify RNA targets, for example, in a single step without the use of a separate reverse transcriptase. Non-limiting examples of polymerases that possess reverse transcriptase capabilities include Bst (large fragment), 9°N DNA polymerase, 9°Nm™ DNA polymerase, Therminator™, Therminator™ II, and the like). In some embodiments, amplification reaction components comprise one or more separate reverse transcriptases. In some embodiments, more than one polymerase is included in in an amplification reaction. For example, an amplification reaction may comprise a polymerase having reverse transcriptase activity and a second polymerase having no reverse transcriptase activity.

In some embodiments, one or more polymerases having exonuclease activity are used during amplification. In some embodiments, one or more polymerases having no or low exonuclease activity are used during amplification. In some embodiments, a polymerase having no or low exonuclease activity comprises one or more modifications (e.g., amino acid substitutions) that reduce or eliminate the exonuclease activity of the polymerase. For example, a modified polymerase having low exonuclease activity can have 10% or less exonuclease activity compared to an unmodified polymerase, for example less than about 10%, 9%, 8%, 7%, 6%, 5%, 4%, 3%, 2%, or 1% exonuclease activity compared to an unmodified polymerase. In some embodiments, a polymerase has no or low 5′ to 3′ exonuclease activity, and/or no or low 3′ to 5′ exonuclease activity. In some embodiments, a polymerase has no or low single strand dependent exonuclease activity, and/or no or low double strand dependent exonuclease activity. Non limiting examples of the modifications that can reduce or eliminate exonuclease activity for a polymerase include one or more amino acid substitutions at position 141 and/or 143 and/or 458 of SEQ ID NO: 1 (e.g., D141A, E143A, E143D and A485L), or at a position corresponding to position 141 and/or 143 and/or 458 of SEQ ID NO: 1.

### Detection and Quantification

The methods described herein can comprise detecting and/or quantifying a nucleic acid amplification product. An amplification product can be detected and/or quantified by any suitable detection and/or quantification method including, for example, any detection method or quantification method described herein. Non-limiting examples of detection and/or quantification methods include molecular beacon (e.g., real-time, endpoint), lateral flow, fluorescence resonance energy transfer (FRET), fluorescence polarization (FP), surface capture, 5′ to 3′ exonuclease hydrolysis probes (e.g., TAQMAN), intercalating/binding dyes, absorbance methods (e.g., colorimetric, turbidity), electrophoresis (e.g., gel electrophoresis, capillary electrophoresis), mass spectrometry, nucleic acid sequencing, digital amplification, a primer extension method (e.g., iPLEX™), Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, AlphaScreen, SNPstream, genetic bit analysis (GBA), Multiplex minisequencing, SNAPshot, GOOD assay, Microarray miniseq, arrayed primer extension (APEX), Microarray primer extension, Tag arrays, Coded microspheres, Template-directed incorporation (TDI), colorimetric oligonucleotide ligation assay (OLA), sequence-coded OLA, microarray ligation, ligase chain reaction, padlock probes, invader assay, hybridization using at least one probe, hybridization using at least one fluorescently labeled probe, cloning and sequencing, the use of hybridization probes and quantitative real time polymerase chain reaction (QRT-PCR), nanopore sequencing, chips and combinations thereof. In some embodiments, detecting a nucleic acid amplification product comprises use of a real-time detection method (i.e., product is detected and/or continuously monitored during an amplification process). In some embodiments, detecting a nucleic acid amplification product comprises use of an endpoint detection method (i.e., product is detected after completing or stopping an amplification process). Nucleic acid detection methods can also employ the use of labeled nucleotides incorporated directly into a target sequence or into probes containing complementary sequences to a target. Such labels can be radioactive and/or fluorescent in nature and can be resolved in any of the manners discussed herein. In some embodiments, quantification of a nucleic acid amplification product can be achieved using one or more detection methods described below. In some embodiments, the detection method can be used in conjunction with a measurement of signal intensity, and/or generation of (or reference to) a standard curve and/or look-up table for quantification of a nucleic acid amplification product.

Detecting a nucleic acid amplification product can comprise use of molecular beacon technology. The term molecular beacon generally refers to a detectable molecule, where the detectable property of the molecule is detectable under certain conditions, thereby enabling the molecule to function as a specific and informative signal. Non-limiting examples of detectable properties include optical properties (e.g., fluorescence), electrical properties, magnetic properties, chemical properties and time or speed through an opening of known size. Molecular beacons for detecting nucleic acid molecules can be, for example, hair-pin shaped oligonucleotides containing a fluorophore on one end and a quenching dye on the opposite end. The loop of the hair-pin can contain a probe sequence that is complementary to a target sequence and the stem is formed by annealing of complementary arm sequences located on either side of the probe sequence. A fluorophore and a quenching molecule can be covalently linked at opposite ends of each arm. Under conditions that prevent the oligonucleotides from hybridizing to its complementary target or when the molecular beacon is free in solution, the fluorescent and quenching molecules are proximal to one another preventing fluorescence resonance energy transfer (FRET). When the molecular beacon encounters a target molecule (e.g., a nucleic acid amplification product), hybridization can occur, and the loop structure is converted to a stable more rigid conformation causing separation of the fluorophore and quencher molecules leading to fluorescence. Due to the specificity of the probe, the generation of fluorescence generally is exclusively due to the synthesis of the intended amplified product. In some instances, a molecular beacon probe sequence hybridizes to a sequence in an amplification product that is identical to or complementary to a sequence in a target nucleic acid. In some instances, a molecular beacon probe sequence hybridizes to a sequence in an amplification product that is not identical to or complementary to a sequence in a target nucleic acid (e.g., hybridizes to a sequence added to an amplification product by way of a tailed amplification primer or ligation).

Molecular beacons are highly specific and can discern a single nucleotide polymorphism. Molecular beacons also can be synthesized with different colored fluorophores and different target sequences, enabling simultaneous detection of several products in the same reaction (e.g., in a multiplex reaction). For quantitative amplification processes, molecular beacons can specifically bind to the amplified target following each cycle of amplification, and because non-hybridized molecular beacons are dark, it is not necessary to isolate the probe-target hybrids to quantitatively determine the amount of amplified product. The resulting signal is proportional to the amount of amplified product. Detection using molecular beacons can be done in real time or as an end-point detection method.

Detecting a nucleic acid amplification product can comprise use of (FRET, the use of surface capture, the use of 5′ to 3′ exonuclease hydrolysis probes (e.g., TAQMAN), the use of intercalating and/or binding dyes, and/or the use of absorbance methods (e.g., colorimetric, turbidity). In some embodiments, detecting a nucleic acid amplification product comprises use of a colorimetric detection method. Any suitable colorimetric detection can be used, and non-limiting examples include assays that use nanoparticles (e.g., metallic nanoparticles, modified nanoparticles, unmodified nanoparticles) and/or peptide nucleic acid (PNA) probes. FRET can be useful for quantifying molecular dynamics, for example, in DNA-DNA interactions as described for molecular beacons. For monitoring the production of a specific product, a probe can be labeled with a donor molecule on one end and an acceptor molecule on the other. Probe-target hybridization brings a change in the distance or orientation of the donor and acceptor and FRET change is observed. Detecting a nucleic acid amplification product via the use of surface capture can be accomplished by the immobilization of specific oligonucleotides to a surface producing a biosensor that is both highly sensitive and selective. Example surfaces that can be used include gold and carbon, and a surface capture method can use a number of covalent or noncovalent coupling methods to attach a probe to the surface. The subsequent detection of a target nucleic acid can be monitored by a variety of methods. In some embodiments, detecting a nucleic acid amplification product comprises use of dyes that specifically stain nucleic acid. For example, intercalating dyes exhibit enhanced fluorescence upon binding to DNA or RNA. Dyes can include DNA or RNA intercalating fluorophores and can include for example, SYTO® 82, acridine orange, ethidium bromide, Hoechst dyes, PicoGreen®, propidium iodide, SYBR® I (an asymmetrical cyanine dye), SYBR® II, TOTO (a thiaxole orange dimer) and YOYO (an oxazole yellow dimer). Dyes provide an opportunity for increasing the sensitivity of nucleic acid detection when used in conjunction with various detection methods.

In some embodiments, detecting a nucleic acid amplification product comprises use of electrophoresis (e.g., gel electrophoresis, capillary electrophoresis), the use of use of mass spectrometry, the use of nucleic acid sequencing, and/or the use of digital amplification (e.g., digital PCR). Mass spectrometry methods include, for example, MALDI, MALDI-TOF, or Electrospray ionization (ESI-MS). These methods can be combined with gas chromatography (GC/MS) and liquid chromatography (LC/MS). Mass spectrometry (e.g., matrix-assisted laser desorption/ionization mass spectrometry (MALDI MS)) can be high throughput due to high-speed signal acquisition and automated analysis off solid surfaces. The entire sequence or a partial sequence of an amplification product can be determined, and the determined nucleotide sequence can be referred to as a read. For example, linear amplification products can be analyzed directly without further amplification in some embodiments (e.g., by using single-molecule sequencing methodology). In some embodiments, linear amplification products are subject to further amplification and then analyzed (e.g., using sequencing by ligation or pyrosequencing methodology). Any suitable sequencing method can be utilized to detect, and in some instances determine the amount of, detectable products generated by the amplification methods described herein. Capillary-gel electrophoresis (CGE) is a combination of traditional gel electrophoresis and liquid chromatography that employs a medium such as polyacrylamide in a narrow bore capillary to generate fast, high-efficient separations of nucleic acid molecules with up to single base resolution. CGE can be combined with laser induced fluorescence (LIF) detection where as few as six molecules of stained DNA can be detected. CGE/LIF detection generally involves the use of fluorescent DNA intercalating dyes including ethidium bromide, YOYO and SYBR® Green 1, and also can involve the use of fluorescent DNA derivatives where fluorescent dye is covalently bound to DNA. Simultaneous identification of several different target sequences (e.g., products from a multiplex reaction) can be made using this method.

### Lysis Buffers

### Lytic Agents

The lysis buffers provided herein can comprise one or more lytic agents (e.g., surfactants, detergents) such as a cationic surfactant, an anionic surfactant, a non-ionic surfactant, and an amphoteric surfactant. The anionic surfactant can comprise NH₄⁺, K⁺, Na⁺, or Li⁺ as a counter ion. The cationic surfactant can comprise I⁻, Br⁻, Cl⁻, or SO₄⁻² as a counter ion. The lysis buffers can further comprise EDTA, EGTA, or the like as a metal ion chelator that forms stronger complexes with heavy metal ions or calcium ion than magnesium ion.

The anionic surfactant can be selected from potassium laurate, triethanolamine stearate, ammonium lauryl sulfate, lithium dodecyl sulfate, sodium lauryl sulfate, sodium alkyl sulfate (C8-16), SDS, alkyl polyoxyethylene sulfate, sodium alginate, dioctyl sodium sulfosuccinate, phosphatidyl glycerol, phosphatidyl inositol, phosphatidylserine, phosphatidic acid and salts thereof, glyceryl ester, sodium carboxymethylcellulose, bile acid and salts thereof, cholic acid, deoxycholic acid, glycocholic acid, taurocholic acid, glycodeoxycholic acid, alkyl sulfonate, aryl sulfonate, alkyl phosphate, alkyl sulfonate, stearic acid and salts thereof, calcium stearate, phosphate, sodium carboxymethyl cellulose, dioctyl sulfosuccinate, dialkyl ester of sodium sulfosuccinic acid, phospholipid and calcium carboxymethyl cellulose.

The cationic surfactant can be, for example, quaternary ammonium compounds, benzalkonium chloride, cetyl trimethyl ammonium bromide, chitonic acid, lauryl dimethyl benzyl ammonium chloride, acyl carnitine hydrochloride, alkyl pyridinium halide, cetylpyridinium chloride, cationic lipids, polymethylmethacrylate trimethyl ammonium bromide, sulfonium compounds, polyvinylpyrrolidone-2-dimethylaminoethyl methacrylate dimethyl sulfate, hexadecyl trimethyl ammonium bromide, phosphonium compounds, quaternary ammonium compounds, benzyl-di(2-chloroethyl)ethyl ammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅-dimethyl hydroxyethyl ammonium chloride, C₁₂₋₁₅-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl(ethenoxy)+ammonium chloride, lauryl dimethyl(ethenoxy) 4 ammonium bromide, N-alkyl (C₁₂₋₁₈)dimethylbenzyl ammonium chloride, N-alkyl (C₁₄₋₁₈)dimethyl-benzyl ammonium chloride, N-tetradecyldimethylbenzyl ammonium chloride monohydrate, dimethyl didecyl ammonium chloride, N-alkyl (C₁₂₋₁₄)dimethyl 1-naphthylmethyl ammonium chloride, trimethylammonium halide alkyl-trimethylammonium salts, dialkyl-dimethylammonium salts, lauryl trimethyl ammonium chloride, ethoxylated alkyamidoalkyldialkylammonium salts, ethoxylated trialkyl ammonium salts, dialkylbenzene dialkylammonium chloride, N-didecyldimethyl ammonium chloride, N-tetradecyldimethylbenzyl ammonium chloride monohydrate, N-alkyl (C₁₂₋₁₄)dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₈₋₁₆ trimethyl ammonium bromide, C₈₋₁₆ trimethyl ammonium chloride, C₁₅ trimethyl ammonium bromide, C₁₇ trimethyl ammonium bromide, dodecylbenzyl triethyl ammonium chloride, polydiallyldimethylammonium chloride, dimethyl ammonium chloride, alkyldimethylammonium halogenide, tricetyl methyl ammonium chloride, decyltrimethylammonium bromide, dodecyltriethylammonium bromide, tetradecyltrimethylammonium bromide, methyl trioctylammonium chloride, POLYQUAT 10, tetrabutylammonium bromide, benzyl trimethylammonium bromide, choline ester, benzalkonium chloride, stearalkonium chloride, cetyl pyridinium bromide, cetyl pyridinium chloride, halide salts of quaternized polyoxyethylalkylamines, MIRAPOL Alkaquat, alkyl pyridinium salts, amine, amine salts, imide azolinium salts, protonated quaternary acrylamides, methylated quaternary polymers, cationic gua gum, benzalkonium chloride, dodecyl trimethyl ammonium bromide, triethanolamine, or poloxamine.

The non-ionic surfactant can be, for example, polyoxyethylene fatty alcohol ether, polyoxyethylene sorbitan fatty acid ester, polyoxyethylene fatty acid ester, polyoxyethylene alkyl ether, polyoxyethylene castor oil derivatives, sorbitan ester, glyceryl ester, glycerol monostearate, polyethylene glycol, polypropylene glycol, polypropylene glycol ester, cetyl alcohol, cetostearyl alcohol, stearyl alcohol, aryl alkyl polyether alcohol, polyoxyethylene polyoxypropylene copolymers, poloxamer, poloxamine, methylcellulose, hydroxycellulose, hydroxymethylcellulose, hydroxyethylcellulose, hydroxypropylcellulose, hydroxypropylmethylcellulose, hydroxypropylmethylcellulose phthalate, noncrystalline cellulose, polysaccharides, starch, starch derivatives, hydroxyethyl starch, polyvinyl alcohol, triethanolamine stearate, amine oxide, dextran, glycerol, gum acacia, cholesterol, tragacanth, or polyvinylpyrrolidone.

The non-ionic surfactant can be, for example, alkyl sulfates, alkyl sulfonates, fatty acid soaps, salts of hydrox-, hydroperoxy-, polyhydroxy-, epoxy-fatty acids, salts of mono- and polycarboxylic acids, prostanoic acid and prostaglandins, leukotrienes and lipoxines, alkyl phosphates, alkyl phosphonates, sodium-dialkyl sufosuccinate, n-alkyl ethoxylated sulfates, cholate and desoxycholate of bile salts, perfluorocarboxylic acids, fluoroacliphatic phosphonates, or fluoroaliphatic sulphates.

The lytic agents provided herein can be capable of acting as a denaturing agent. “Denaturing agent” or “denaturant,” as used herein, shall be given its ordinary meaning and include any compound or material which will cause a reversible unfolding of a protein. The strength of a denaturing agent or denaturant will be determined both by the properties and the concentration of the particular denaturing agent or denaturant. Suitable denaturing agents or denaturants may be chaotropes, detergents, organic solvents, water miscible solvents, phospholipids, or a combination of two or more such agents. Suitable chaotropes include, but are not limited to, urea, guanidine, and sodium thiocyanate. Useful detergents may include, but are not limited to, strong detergents such SDS, or polyoxyethylene ethers (e.g. Tween or Triton detergents), Sarkosyl, mild non-ionic detergents (e.g., digitonin), mild cationic detergents such as N->2,3-(Dioleyoxy)-propyl-N,N,N-trimethylammonium, mild ionic detergents (e.g. sodium cholate or sodium deoxycholate) or zwitterionic detergents including, but not limited to, sulfobetaines (Zwittergent), 3-(3-chlolamidopropyl)dimethylammonio-1-propane sulfate (CHAPS), and 3-(3-chlolamidopropyl)dimethylammonio-2-hydroxy-1-propane sulfonate (CHAPSO). Organic, water miscible solvents such as acetonitrile, lower alkanols (especially C₂-C₄ alkanols such as ethanol or isopropanol), or lower alkandiols (especially C₂-C₄ alkandiols such as ethylene-glycol) may be used as denaturants. Phospholipids can be naturally occurring phospholipids such as phosphatidylethanolamine, phosphatidylcholine, phosphatidylserine, and phosphatidylinositol or synthetic phospholipid derivatives or variants such as dihexanoylphosphatidylcholine or diheptanoylphosphatidylcholine.

Suitable surfactant levels can be from about 0.1% to about 25%, from about 0.25% to about 10%, or from about 0.5% to about 5% by weight of the total composition. In some embodiments, the surfactants are anionic surfactants, amphoteric surfactants, nonionic surfactants, zwitterionic surfactants, cationic surfactants, and mixtures thereof. In some embodiments, it can be advantageous to use anionic, amphoteric, nonionic and zwitterionic surfactants (and mixtures thereof).

Useful anionic surfactants herein include the water-soluble salts of alkyl sulphates and alkyl ether sulphates having from 10 to 18 carbon atoms in the alkyl radical and the water-soluble salts of sulphonated monoglycerides of fatty acids having from 10 to 18 carbon atoms. Sodium lauryl sulphate and sodium coconut monoglyceride sulphonates are examples of anionic surfactants of this type.

Suitable cationic surfactants can be broadly defined as derivatives of aliphatic quaternary ammonium compounds having one long alkyl chain containing from about 8 to 18 carbon atoms such as lauryl trimethylammonium chloride; cetyl pyridinium chloride; benzalkonium chloride; CTAB; di-isobutylphenoxyethyl-dimethylbenzylammonium chloride; coconut alkyltrimethyl-ammonium nitrite; cetyl pyridinium fluoride; etc. Certain cationic surfactants can also act as germicides in the compositions disclosed herein.

Suitable nonionic surfactants that can be used in the compositions, methods and kits of the present disclosure can be broadly defined as compounds produced by the condensation of alkylene oxide groups (hydrophilic in nature) with an organic hydrophobic compound which may be aliphatic and/or aromatic in nature. Examples of suitable nonionic surfactants include the poloxamers; sorbitan derivatives, such as sorbitan di-isostearate; ethylene oxide condensates of hydrogenated castor oil, such as PEG-30 hydrogenated castor oil; ethylene oxide condensates of aliphatic alcohols or alkyl phenols; products derived from the condensation of ethylene oxide with the reaction product of propylene oxide and ethylene diamine; long chain tertiary amine oxides; long chain tertiary phosphine oxides; long chain dialkyl sulphoxides and mixtures of such materials. These materials are useful for stabilizing foams without contributing to excess viscosity build for the consumer product composition.

Zwitterionic surfactants can be broadly described as derivatives of aliphatic quaternary ammonium, phosphonium, and sulphonium compounds, in which the aliphatic radicals can be straight chain or branched, and wherein one of the aliphatic substituents contains from about 8 to 18 carbon atoms and one contains an anionic water-solubilizing group, e.g., carboxy, sulphonate, sulphate, phosphate or phosphonate.

Exemplary anionic, single-chain surface active agents include alkyl sulfates, alkyl sulfonates, alkyl benzene sulfonates, and saturated or unsaturated fatty acids and their salts. Moieties comprising the polar head group in the cationic surfactant can include, for example, quaternary ammonium, pyridinium, sulfonium, and/or phosphonium groups. For example, the polar head group can include trimethylammonium. Exemplary cationic, single-chain surface active agents include alkyl trimethylammonium halides, alkyl trimethylammonium tosylates, and N-alkyl pyridinium halides.

Alkyl sulfates can include sodium octyl sulfate, sodium decylsulfate (SDeS), SDS, and sodium tetra-decyl sulfate. Alkyl sulfonates can include sodium octyl sulfonate, sodium decyl sulfonate, and sodium dodecyl sulfonate. Alkyl benzene sulfonates can include sodium octyl benzene sulfonate, sodium decyl benzene sulfonate, and sodium dodecyl benzene sulfonate. Fatty acid salts can include sodium octanoate, sodium decanoate, sodium dodecanoate, and the sodium salt of oleic acid.

Alkyl trimethylammonium halides can include octyl trimethylammonium bromide, decyl trimethylammonium bromide, dodecyl trimethylammonium bromide, myristyl trimethylammonium bromide, and CTAB. Alkyl trimethylammonium tosylates can include octyl trimethylammonium tosylate, decyl trimethylammonium tosylate, dodecyl trimethylammonium tosylate, myristyl trimethylammonium tosylate, and cetyl trimethylammonium tosylate. For example, N-alkyl pyridinium halides can include decyl pyridinium chloride, dodecyl pyridinium chloride, cetyl pyridinium chloride, decyl pyridinium bromide, dodecyl pyridinium bromide, cetyl pyridinium bromide, decyl pyridinium iodide, dodecyl pyridinium iodide, cetyl pyridinium iodide.

The cationic surfactant can comprise at least one compound selected from dodecyltrimethylammonium bromide, tetradecyltrimethylammonium bromide, cetyltrimethylammonium bromide, cetyldimethylethylammonium bromide, (C1 to C30 alkyl)-trimethylammonium bromide, a (C1 to C30 alkyl)amine, a (C1 to C30 alkyl) imidazoline, ethoxylated amine, a quaternary compound, a quaternary ester, a (C1 to C30 alkyl)amine oxide, lauramine oxide, dicetyldimonium chloride, cetrimonium chloride, a primary polyethoxylated fatty amine salt, a secondary polyethoxylated fatty amine salt, a tertiary polyethoxylated fatty amine salt, a quaternary ammonium salt, a tetra(C1 to C30 alkyl) ammonium halide, a (C1 to C30 alkyl)amide-(C1 to C30-alkyl) ammonium halide, a tri (C1 to C30 alkyl)benzylammonium halide, a tri (C1 to C30 alkyl) hydroxy-(C1 to C30 alkyl) ammonium halide, a (C1 to C30 alkyl)pyridinium chloride, a (C1 to C30 alkyl)pyridinium bromide, and a amine oxide.

The anionic surfactant can comprise at least one compound selected from SDS, a (C6 to C30 alkyl)benzene sulfonate, a C6 to C30 alpha olefin sulfonate, a paraffin sulfonate, a (C6 to C30 alkyl) ester sulfonate, a (C6 to C30 alkyl) sulfate, a (C6 to C30 alkyl alkoxy) sulfate, a (C6 to C30 alkyl) sulfonate, a (C6 to C30 alkyl alkoxy) carboxylate, a (C6 to C30 alkyl alkoxylated) sulfate, a mono(C1 to C30 alkyl) (ether)phosphate, a di(C6 to C30 alkyl) (ether)phosphate, a (C6 to C30 alkyl) sarcosinate, a sulfosuccinate, sodium bis(2-ethylhexyl) sulfosuccinate, ethoxylate 4-nonylphenyl ether glycolic acid, a (C1 to C30 alkyl) isethionate, taurate, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl phosphate, sodium tridecyl phosphate, sodium behenyl phosphate, sodium laureth-2 phosphate, sodium ceteth-3 phosphate, sodium trideceth-4 phosphate, sodium dilauryl phosphate, sodium ditridecyl phosphate, sodium ditrideceth-6 phosphate, sodium lauroyl sarcosinate, lauroyl sarcosine, cocoyl sarcosine, ammonium cocoyl sulfate, sodium cocoyl sulfate, sodium trideceth sulfate, sodium tridecyl sulfate, ammonium trideceth sulfate, ammonium tridecyl sulfate, sodium cocoyl isethionate, disodium laureth sulfosuccinate, sodium methyl oleoyl taurate, sodium laureth carboxylate, sodium trideceth carboxylate, sodium lauryl sulfate, potassium cocoyl sulfate, potassium lauryl sulfate, monoethanolamine cocoyl sulfate, sodium tridecyl benzene sulfonate, sodium dodecyl benzene sulfonate, and SDS.

The non-ionic surfactant can comprise, for example, a C6 to C18 alkyl alcohol, a (C6 to C18 alkyl) phenol, a (C6 to C18 alkyl) ethoxylate, a (C6 to C18 alkyl) phenol (C1 to C3 alkoxylate), a block oxy (C1 to C3 alkylene) condensate of a C6 to C18 alkyl phenol, an oxy (C1 to C3 alkylene) condensate of alkanol, an oxyethylene/oxypropylene block copolymer, an amine oxide, a phosphine oxide, an alkylamine oxide having 8 to 50 carbon atoms, a mono or di(C8 to C30)alkyl alkanolamide, a (C6 to C30 alkyl) polysaccharide, a sorbitan fatty acid ester, a polyoxyethylene sorbitan fatty acid ester, a polyoxyethylene sorbitol ester, a polyoxyethylene nonylphenyl ether, a polyoxyethylenic acid, a polyoxyethylene alcohol, a coco monoethanolamide, a coco diethanolamide, a coco diglycoside, a (C8 to C30 alkyl) polyglycoside, cocamidopropyl, lauramine oxide, polyoxyethylene (20) sorbitan monolaurate, an ethoxylated linear C8 to C30 alcohol, cetearyl alcohol, lanolin alcohol, stearic acid, glyceryl stearate, polyethylene glycol 100 stearate, 4-(1,1,3,3-tetramethylbutyl)phenyl polyethylene glycol, polyoxyethylene (10) cetyl ether, eicosaethylene glycol octadecyl ether, and HO(CH₂CH₂O)₂₀(CH₂CH(CH₃)O)₇₀(CH₂CH₂O)₂₀H.

### Reagent Composition

The reagent compositions described herein (e.g., dried composition) can be provided in a “dry form,” or in a form not suspended in liquid medium. The “dry form” of the compositions can include dry powders, lyophilized compositions, spray-dried, or precipitated compositions. The “dry form” compositions can include one or more lyoprotectants, such as sugars and their corresponding sugar alcohols, such as sucrose, lactose, trehalose, dextran, erythritol, arabitol, xylitol, sorbitol, and mannitol; amino acids, such as arginine or histidine; lyotropic salts, such as MgSO₄; polyols, such as propylene glycol, glycerol, poly(ethylene glycol), or polypropylene glycol); and combinations thereof. Additional exemplary lyoprotectants include gelatin, dextrins, modified starch, and carboxymethyl cellulose. As used herein, the terms “lyophilization,” “lyophilized,” and “freeze-dried” refer to a process by which the material to be dried is first frozen and then the ice or frozen solvent is removed by sublimation in a vacuum environment. “Lyophilisate” refers to a lyphophilized substance. As disclosed herein, the dried composition can comprise one or more additives and one or more amplification reagents. The compositions described herein (e.g., wet composition) can be provided in a “wet form,” or in a form suspended in liquid medium.

The dried composition can be frozen or lyophilized or spray dried. The dried composition can be heat dried. The dried composition can comprise one or more additives (e.g., a polymer, a sugar or sugar alcohol). The sugar or sugar alcohol can comprise sucrose, lactose, trehalose, dextran, erythritol, arabitol, xylitol, sorbitol, mannitol, or any combination thereof. The polymer can comprise polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof. The one or more additives can comprise one or more amino acids. The one or more additives can comprise Tween 80, Tween 20 and/or Triton X-100. In some embodiments, the one or more additives help lyophilization of the reaction compositions and/or the dissolution of dried pellets. The one or more additives can comprise a nonionic detergent at a concentration of about 0.01% in the dried composition (e.g., dried pellet).

The frozen or lyophilized or spray dried or heat dried composition or the aqueous composition for preparing the frozen or lyophilized or spray dried composition may comprise one or more of the following: (i) Non-aqueous solvents such as ethylene glycol, glycerol, dimethylsulphoxide, and dimethylformamide. (ii) Surfactants such as Tween 80, Brij 35, Brij 30, Lubrol-px, Triton X-10; Pluronic F127 (polyoxyethylene-polyoxypropylene copolymer) also known as poloxamer, poloxamine, and SDS. (iii) Dissacharides such as trehalose, sucrose, lactose, and maltose. (iv) Polymers (which may have different MWs) such as polyethylene glycol, dextran, polyvinyl alcohol), hydroxypropyl methylcellulose, gelatin, polyvinylpyrrolidone, hydroxyethyl cellulose, Ficoll, and albumin. (v) Amino acids such as glycine, proline, 4-hydroxyproline, L-serine, glutamate, alanine, lysine, sarcosine, and gamma-aminobutyric acid.

### Kits

Disclosed herein include kits for detecting a target nucleic acid sequence in a sample. In some embodiments, the kit comprises: a lysis buffer comprising one or more lytic agents (e.g., one or more surfactants), wherein the one or more lytic agents are capable of lysing biological entities to release sample nucleic acids comprised therein, wherein the sample nucleic acids are suspected of comprising a target nucleic acid sequence. The lysis buffer can comprise one or more surfactants, (NH₄)₂SO₄, MgSO₄, a chelator, acid(s), alcohols, pH buffer(s), and/or tween surfactant(s). The kit can comprise a reagent composition (e.g., a wet composition, a dried composition) comprising one or more amplification reagents, wherein the one or more amplification reagents comprise one or more components for amplifying a target nucleic acid sequence under isothermal amplification conditions. In some embodiments, said components comprise: (i) a first primer and a second primer, wherein the first primer is capable of hybridizing to a sequence of a first strand of the target nucleic acid sequence, and the second primer is capable of hybridizing to a sequence of a second strand of the target nucleic acid sequence; and (ii) an enzyme having a hyperthermophile polymerase activity capable of generating a nucleic acid amplification product.

The kit can comprise: at least one component providing real-time detection activity for a nucleic acid amplification product. The real-time detection activity can be provided by a molecular beacon. The reagent composition can comprise a reverse transcriptase and/or a reverse transcription primer.

Kits can comprise, for example, one or more polymerases and one or more primers, and optionally one or more reverse transcriptases and/or reverse transcription primers, as described herein. Where one target is amplified, a pair of primers (forward and reverse) can be included in the kit. Where multiple target sequences are amplified, a plurality of primer pairs can be included in the kit. A kit can include a control polynucleotide, and where multiple target sequences are amplified, a plurality of control polynucleotides can be included in the kit. In some embodiments, the enzyme having a hyperthermophile polymerase activity has an amino acid sequence that is at least about 90% or 95% identical to the amino acid sequence of SEQ ID NO: 1 or a functional fragment thereof. The enzyme having a hyperthermophile polymerase activity can be a polymerase comprising the amino acid sequence of SEQ ID NO: 1.

The nucleic acid amplification product can be about 20 to 40 bases long. The nucleic acid amplification product can comprise: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

The biological entities can comprise one or more of prokaryotic cells, eukaryotic cells, viral particles, exosomes, protoplasts, and microvesicles. The biological entities can comprise a virus, a bacteria, a fungi, a protozoa, portions thereof, or any combination thereof. The target nucleic acid sequence can be a nucleic acid sequence of a virus, bacteria, fungi, or protozoa. The sample nucleic acids can be derived from a virus, bacteria, fungi, or protozoa.

Kits can also comprise one or more of the components in any number of separate vessels, chambers, containers, packets, tubes, vials, microtiter plates and the like, or the components can be combined in various combinations in such containers. Components of the kit can, for example, be present in one or more containers. In some embodiments, all of the components are provided in one container. In some embodiments, the enzymes (e.g., polymerase(s) and/or reverse transcriptase(s)) can be provided in a separate container from the primers. The components can, for example, be lyophilized, heat dried, freeze dried, or in a stable buffer. In some embodiments, polymerase(s) and/or reverse transcriptase(s) are in lyophilized form or heat dried form in a single container, and the primers are either lyophilized, heat dried, freeze dried, or in buffer, in a different container. In some embodiments, polymerase(s) and/or reverse transcriptase(s), and the primers are, in lyophilized form or heat dried form, in a single container.

Kits can further comprise, for example, dNTPs used in the reaction, or modified nucleotides, vessels, cuvettes or other containers used for the reaction, or a vial of water or buffer for re-hydrating lyophilized or heat-dried components. The buffer used can, for example, be appropriate for both polymerase and primer annealing activity.

Kits can also comprise instructions for performing one or more methods described herein and/or a description of one or more components described herein. Instructions and/or descriptions can be in printed form and can be included in a kit insert. A kit also can include a written description of an internet location that provides such instructions or descriptions.

Kits can further comprise reagents used for detection methods, for example, reagents used for FRET, lateral flow devices, dipsticks, fluorescent dye, colloidal gold particles, latex particles, a molecular beacon, or polystyrene beads.

### EXAMPLES

Some aspects of the embodiments discussed above are disclosed in further detail in the following examples, which are not in any way intended to limit the scope of the present disclosure.

### Example 1

### Lysis Buffer Stability Testing

This example provides the results of stability testing of a lysis buffer for use with the disclosed nucleic acid detection methods. The composition of an initial DNA assay lysis buffer (DALB; Table 2) and RNA assay lysis buffer (RALB; Table 3) are shown below.

TABLE 2
DNA Assay Lysis Solution - DALB

| Buffer component | [1X] | Units | Purpose |
|---|---|---|---|
| dH₂O | — | — | |
| (NH₄)₂SO₄ | 5.0 | mM | Salt for primer specificity |
| MgSO₄ | 4.0 | mM | Enzyme cofactor (Mg⁺²) |
| glycine | 10.0 | mM | Buffer pH |
| HCl | 8.8 | mM | pH 2.2 |
| SDS | 0.20 | % | Ionic surfactant for cell lysis |

TABLE 3
RNA Assay Lysis Solution - RALB

| Buffer component | [1X] | Units | Purpose |
|---|---|---|---|
| dH₂O | — | — | |
| (NH₄)₂SO₄ | 5.0 | mM | Salt for primer specificity |
| MgSO₄ | 4 | mM | Enzyme cofactor (Mg⁺²) |
| CTAB | 0.20 | % | Ionic surfactant for cell lysis |

Stability testing and opacity (precipitate) screening was performed at several temperatures. Besides testing at room temperature (RT), in order to accelerate screening of candidate formulations, formulations were also aged at 4° C. (an accelerated temperature) in addition to 14° C. (an intermediate accelerated temperature). A visual examination for opacity/precipitation found that the initial batch of DNA assay lysis buffer experienced rapid precipitation (over several days) when stored and RT and 4° C.

A series of test mixtures were prepared and evaluated after mixing to identify the root cause for the observed opacity (precipitation). Without being bound by theory, the possibility that the precipitate is complex of Mg⁺² and anionic SDS was explored. Based on the results shown in Table 4, it was determined that the opacity (precipitate) is caused by the interaction of the MgSO₄ with the SDS. These results confirm that the precipitate is complex of Mg⁺² and SDS and that precipitation occurs regardless of pH.

TABLE 4
Precipitate Determination

| | | | Observation after |
|---|---|---|---|
| Solution | Components | Notes | 3 days at RT |
| 1 | SDS, glycine-HCL | Does low pH affect | No precipitate |
| | | SDS solubility | |
| 2 | MgSO₄, | Does low pH affect | No precipitate |
| | glycine-HCL | Mg solubility | |
| 3 | MgSO₄, | Is precipitant result | Precipitate |
| | SDS | of Mg/SDS interaction | |
| 4 | MgSO₄, SDS, | Does low pH affect | Precipitate |
| | glycine-HCL | Mg/SDS | |

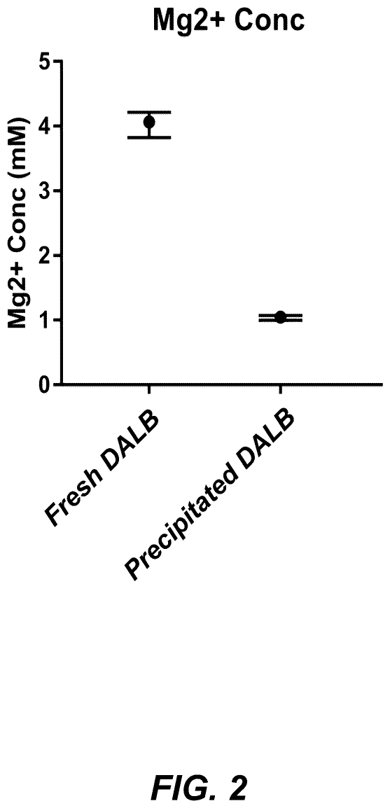

FIG. 2 depicts data related to a BioAssay colorimetric Magnesium assay on fresh DNA assay lysis solution (DALB) at time of production and precipitated DNA assay lysis solution (without disturbing precipitant) 7 days later. A steep decline in the concentration of enzyme cofactor Mg⁺² was found in the precipitated DNA assay lysis solution after only seven days. The DNA assay is sensitive to MgSO₄ as this is the cofactor for the polymerase, and this component is not considered an adjustable parameter for stability. As seen in Table 5, decreasing SDS 4-fold did not improve stability (all other components at nominal levels). It was therefore reasoned that no ‘small’ concentration change is likely to yield significant improvement.

TABLE 5
Effect of SDS Concentration on Precipitation

| SDS, % | 4° C. | 14° C. | RT (18 Month Target) |
|---|---|---|---|
| 0.2 | 1 | * | 1-4 |
| 0.1 | 2 | 1 | 3-4 |
| 0.05 | 2 | 1 | 2 |

Precipitate Observed after # Days
*Not Tested

Without being bound by any particular theory, the relationship between Mg and SDS concentrations with precipitation was explored. Dilutions of lysis buffer show that more than 4-fold dilution is needed to maintain opacity (Table 6). It is noted this data differs the experiment of Table 5 in that the full buffer diluted (not just SDS). This implies a signification decrease in concentration of Mg/SDS is required for a non-opaque lytic buffer formulation.

TABLE 6
Precipitation Observation Results at 4° C. (Visual)

| DALB | Day 1 | Day 3 | Day 7 |
|---|---|---|---|
| 1x | + | + | + |
| 1/2x | + | + | + |
| 1/4x | − | + | + |
| 1/10x | − | − | − |
| 1/100x | − | − | − |
| 1/1000x | − | − | − |
| 1/2000x | − | − | − |

### Example 2

### Evaluating a First Set of Approaches to Mitigate Precipitation

This example provides the results of various approaches and compositions tested for the mitigation of precipitant formation in lysis buffers.

The effect of changing the order of reagent addition on the formation of precipitant was tested. The addition of MgSO₄ was made last to see if this would mitigate precipitation. However, the reordered protocol was not found to improve stability.

The effect of substituting a cationic surfactant for SDS was tested. Without being bound by theory, the possibility that this approach would avoid the charged interaction between Mg and SDS was explored. As seen in Table 7, lysis solution with CTAC shows good stability across all temps, and CTAB did precipitate at 4° C., but better than SDS at 14° C. and RT.

TABLE 7
Lysis Buffer Stability with Cationic Surfactants

| | | | RT |
|---|---|---|---|
| Buffer | 4° C. | 14° C. | (18 Month Target) |
| w/SDS | 1 | * | 1-4 |
| w/CTAB | 3 | 142 | 150 |
| w/CTAC | 66 | 60 | 66 |

No Precipitate Observed in # Days
Precipitate Observed after # Days
*Not Tested

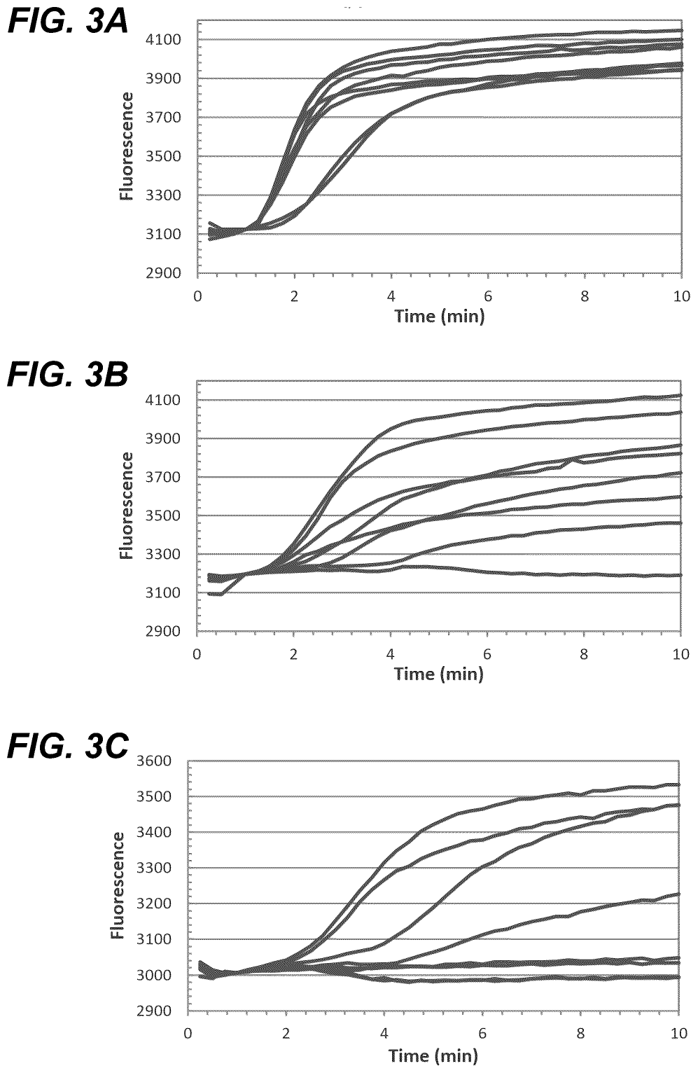

In spite of the improved lysis buffer stability observed with the use of CTAB, DNA assay performance was found to be negatively affected by CTAB. Substituting CTAB in lysis buffer negatively affected Group A Strep assay performance (FIGS. 3A-3C). FIGS. 3A-3C depict data related to the effect of CTAB substitution in lysis buffer on Group A Strep assay performance. Assays with standard lysis buffer (FIG. 3A) or with lysis solutions with 0.2% CTAB (FIGS. 3B-3C) are shown. Fluorescence (FAM) versus time (min) is depicted for assays with 50 cp/reaction. Without being bound by theory, the cationic surfactant may stabilize double-stranded DNA, hence the target is less likely to be denatured to enable the first priming events.

The effect of employing an alternative counter ion (e.g., swap MgSO₄ for MgX) on the formation of precipitant was tested. The literature suggests anion affects salting out or in of SDS as follows: SO₄2->OH->F->Cl->NO₃->I->SCN-De. However, no significant benefit as observed with MgCl₂ or MgI₂, as neither MgCl₂ or MgI₂ improved stability at low temperatures (Table 8). MgI₂ needed to be filtered to remove particulate. Note that after formulation, the Mg assayed low for MgI₂ and adjusted.

TABLE 8
Lysis Buffer Stability with Alternative Counter Ions

| Buffer | 4° C. | 14° C. | RT (18 Month Target) |
|---|---|---|---|
| w/MgCl₂ | 1 | * | 1 |
| w/MgI₂ | 2 | 1 | 127 |
| w/MgSO₄ | 1 | * | 1-4 |

No Precipitate Observed in # Days
Precipitate Observed after # Days
*Not Tested

The effect of employing alternative anionic surfactants was tested. The literature suggests sodium 1-dodecanesulphonate is less apt to complex with Mg than SDS, and also that sodium 1-dodecanesulphonate may bind more weakly to Mg ions than SDS. However, sodium 1-dodecanesulphonate at 0.2% went cloudy immediately when MgSO₄ added, and the final lysis buffer precipitated immediately at RT. Therefore, sodium 1-dodecanesulphonate is not a suitable option. Next, the use of lithium dodecyl sulfate was considered, as lithium dodecyl sulfate is stable at low temperatures and its use as a lysing agent was suggested by some literature. However, low temperature storage was recommended for LDS in solution, and this alternative surfactant was not pursued based on recommendation to store solutions at −20° C.

### Example 3

### Evaluating a Second Set of Approaches to Mitigate Precipitation

This example provides approaches and compositions that mitigate formation of precipitant in lysis buffers.

### Increasing Ammonium Sulfate Concentration

The effect of increasing (NH₄)₂SO₄ concentration on lysis buffer stability investigated. Without being bound by any particular theory, this approach could improve lysis buffer stability via the salt effect based upon Le Chatelier's Principle of Equilibria. 5 mM is nominal (NH₄)₂SO₄ concentration, increased (NH₄)₂SO₄ was found to have impact on opacity at 14° C. and at room temperature (Table 9). Testing of assay impact found a benefit to increasing (NH₄)₂SO₄ concentration. In some embodiments provided herein, increased (NH₄)₂SO₄ is combined with one or more of the other approaches described herein (e.g., alcohol addition) to increase lysis buffer stability. In some embodiments, such a combination-based approach yields a synergistic improvement in lysis buffer stability.

TABLE 9
Lysis Buffer Stability with Varying
Ammonium Sulfate Concentrations

| SDS, % | (NH₄)₂SO₄, mM | 4° C. | 14° C. | RT |
|---|---|---|---|---|
| 0.2 | 5 | 1 | * | 1-4 |
| | 10 | 1 | 2 | 150 |
| | 30 | 1 | 142 | 150 |
| | 60 | 1 | 142 | 150 |
| 0.1 | 5 | 2 | 1 | 3-4 |
| | 10 | 2 | 1 | 8 |
| | 30 | 2 | 142 | 149 |
| | 60 | 2 | 142 | 149 |
| 0.05 | 5 | 2 | 1 | 2 |
| | 10 | 2 | 2 | 19 |
| | 30 | 2 | 142 | 149 |
| | 60 | 2 | 142 | 149 |

No Precipitate Observed in # Days
Precipitate Observed after # Days
*Not Tested

### Solvents Improving SDS Solubility

The effect of adding solvents (to improve SDS solubility) on lysis buffer stability was investigated.

The addition of DMSO yielded incremental improvements in precipitate reduction but did not provide stability at 4° C. (Table 10).

TABLE 10
Lysis Buffer Stability with DMSO Inclusion

| SDS | DMSO % | 4° C. | 14° C. | RT |
|---|---|---|---|---|
| 0.2 | 0 | 1 | * | 1-4 |
| | 1 | 1 | 1 | 143 |
| | 5 | 1 | 2 | 143 |
| | 10 | 1 | 142 | 143 |

No Precipitate Observed in # Days
Precipitate Observed after # Days
*Not Tested

Various alcohols were tested on their effects on increasing lipid solvency, and thereby improving SDS solubility. In some embodiments, optimal assay temperature is not affected by alcohol inclusion in the lysis buffer. In some embodiments, the alcohol inclusion has little or no impact on Archaeal Polymerase Amplification (APA).

The addition of ethanol and isopropanol yielded improvements in lysis buffer stability but precipitate formation occurred at 4° C. even at high concentrations (Tables 10 and 11, respectively).

TABLE 11
Lysis Buffer Stability with Ethanol Inclusion

| SDS | Ethanol % | 4° C. | 14° C. | RT |
|---|---|---|---|---|
| 0.2 | 0 | 1 | * | 1-4 |
| | 1 | 2 | 3-4 | 13 |
| | 5 | 1 | 7 | 143 |
| | 10 | 1 | 143 | 143 |

No Precipitate Observed in # Days
Precipitate Observed after # Days
*Not Tested

TABLE 12
Lysis Buffer Stability with Isopropanol Inclusion

| SDS | Isopropanol % | 4° C. | 14° C. | RT |
|---|---|---|---|---|
| 0.2 | 0 | 1 | * | 1-4 |
| | 2 | 2-3 | 5 | 100 |
| | 3 | 2-3 | 4 | 100 |
| | 4 | 3 | 95 | 95 |
| | 5 | 3 | 95 | 95 |

No Precipitate Observed in # Days
Precipitate Observed after # Days
*Not Tested

Given the slightly better performance observed with isopropanol relative to ethanol (compare Table 11 to Table 12), longer chain alcohols were next tested. Inclusion of 3% Isobutyl Alcohol (IBA) in the lysis buffer notably improved stability and lysis buffers comprising 4% IBA did not form precipitates at any tested condition (Table 13). The addition of pentanol was also found to improve lysis buffer stability (Table 14).

TABLE 13
Lysis Buffer Stability with Isobutyl Alcohol Inclusion

| IBA, % | 4° C. | | | 14° C. | | RT | |
|---|---|---|---|---|---|---|---|
| 0 | 1 | * | * | * | * | 1-4 | * |
| (Unmodified DALB) | | | | | | | |
| 1 | 3 | * | * | 130 | * | 130 | * |
| 2 | 2 | 5 | * | 108 | 60 | 108 | 71 |
| 3 | 36 | 7 | 21 | 108 | 60 | 108 | 74 |
| 4 | 116 | 74 | * | 108 | * | 108 | * |

No Precipitate Observed in # Days
Precipitate Observed after # Days
* Not Tested

TABLE 14
Lysis Buffer Stability with Pentanol Inclusion

| Pentanol, % | 4° C. | 14° C. | RT |
|---|---|---|---|
| 0 | 1 | * | 1-4 |
| (unmodified DALB) | | | |
| 1 | 16 | 60 | 60 |
| 2 | 60 | 60 | 60 |
| 3 | ** | ** | ** |

No Precipitate Observed in # Days
Precipitate Observed after # Days
*Not Tested
**Took several days to go into solution

Hexanol was found to be hard to get into the lysis solution even at low concentrations. Based the studies described herein, the solubility of alcohols in water and improved solubility of SDS appears to correlate with carbon chain length (e.g., Ethanol (2C)<Isopropanol (3C)<Isobutyl Alcohol (4C)<Pentanol (5C)<Hexanol (6C)), with increasing solubility in water with decreasing carbon chain length, and increasing lipid (SDS) solubility with increasing carbon chain length.

The effect of combining IBA addition with increased (NH₄)₂SO₄ on lysis buffer stability was next investigated. Improved stability with IBA combined with increased (NH₄)₂SO₄ (Table 16) relative to IBA inclusion alone (Table 15). In some embodiments, the combination of IBA with increased (NH₄)₂SO₄ does not impact the downstream assay performance. In some embodiments, the increased (NH₄)₂SO₄ enables increased lysis buffer stability at lower alcohol concentrations.

TABLE 15
Lysis Buffer Stability with Isobutyl Alcohol Inclusion

| IBA, % | 4° C. | | |
|---|---|---|---|
| 0 | 1 | * | * |
| (unmodified DALB) | | | |
| 1 | 3 | * | * |
| 2 | 2 | 5 | * |
| 3 | 36 | 7 | 21 |
| 4 | 116 | 74 | * |

No Precipitate Observed in # Days
Precipitate Observed after # Days
*Not Tested

TABLE 16
Lysis Buffer Stability with Isobutyl Alcohol
and Ammonium Sulfate Inclusion

| IBA, % | (NH₄)₂SO₄, mM | 4° C. |
|---|---|---|
| 1 | 5*** | 3 |
| | 10 | 7 |
| | 20 | 9-10 |
| 2 | 5*** | 2 |
| | 10 | 19 |
| | 20 | 105 |

No Precipitate Observed in # Days
Precipitate Observed after # Days
***Unmodified DALB contains 5 mM (NH₄)₂SO₄.

### Alternative Surfactants

The use of sodium decyl sulfate (SDeS) and sodium octyl sulfate (SOctylS) as alternative surfactants was also tested. While the literature suggests SDeS and sodium octyl sulfate are not as lytic as SDS, these surfactants were tested at different pH and temperatures than the nucleic acid detection methods and compositions described herein. Both of these alternative surfactants yielded significant improvements in lysis buffer stability relative to SDS (Table 17). In some embodiments, these alternative surfactants do not substantially impact lysis and assay performance as compared to SDS.

TABLE 17
Lysis Buffer Stability with Alternative Surfactants

| Surfactant | | | |
|---|---|---|---|
| 0.2% in DALB | 4° C. | 14° C. | RT |
| SDS | 1 | * | 1-4 |
| SDecylS | 50 | 50 | 50 |
| SOctylS | 52 | 52 | 52 |

No Precipitate Observed in # Days
Precipitate Observed after # Days
*Not Tested

### Example 4

### Stable DNA Assay Lytic Buffer Formulations and RNA Assay Lytic Buffer Formulations

The composition of and stability data for DNA assay lytic buffer formulations and RNA assay lytic buffer formulations provided herein are shown in Tables 18-19. Substitution of cetyl trimethylammonium chloride (CTAC) for CTAB enhanced RNA assay lytic buffer stability. Substitution of SDeS for SDS and increasing the concentration of (NH₄)₂SO₄ greatly improved DNA assay lytic buffer stability. Tween 80 (Tw80) was added to enhance assay performance in some embodiments.

TABLE 18
Stable RNA Assay Lysis Buffers

| RNA Buffer | Surfactant | (NH₄)₂SO₄ | 4° C. | 14° C. | RT |
|---|---|---|---|---|---|
| RALB | CTAB | 5 mM | 12 | 372 | 433 |
| RALB.v2 | CTAC | 5 mM | 128* | 275 | 275 |

No Precipitate Observed in # Days
Precipitate Observed after # Days
*Floaters (flocculation) observed

TABLE 19
Stable DNA Assay Lysis Buffers

| DNA Buffer | Surfactant | (NH₄)₂SO₄ | 4° C. | 14° C. | RT |
|---|---|---|---|---|---|
| DALB.v2 | SDS | 5 mM | 1 | 2 | 28/32 |
| DALB.v3 | 2X SDS + Tw80 | 10 mM | 1 | 3 | 93 |
| DALB.v4 | SDeS | 5 mM | 280 | 280 | 280 |
| DALB.v5 | SDeS | 10 mM | 167 | 167 | 167 |
| DALB.v6 | 2X SDeS | 10 mM | 114 | 114 | 114 |
| DALB.v7 | 4X SDeS | 10 mM | 114 | 114 | 114 |
| DALB.v8 | 2X SDeS + Tw80 | 10 mM | 93 | 93 | 93 |
| DALB.v9 | CTAB | 5 mM | 3 | 372 | 380 |
| DALB.v10 | CTAC | 5 mM | 149* | 290 | 296 |
| DALB.v11 | S Octyl S | 5 mM | 282 | 282 | 282 |

No Precipitate Observed in # Days
Precipitate Observed after # Days
*Floaters (flocculation) observed
DALB.v3 is the DNA formulation used in the initial assay feasibility study.
DALB.v8 is DALB.v3 with surfactant substitution.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A lysis buffer, comprising:
one or more surfactants;
ammonium sulfate ((NH₄)₂SO₄); and
magnesium sulfate (MgSO₄),
wherein the lysis buffer does not comprise sodium dodecyl sulfate (SDS), cetyl trimethylammonium bromide (CTAB), or both, and
wherein formation of a precipitate in the lysis buffer is substantially inhibited for a period of time under a storage condition.

2. The lysis buffer of claim 1, wherein the appearance of the precipitate in the lysis buffer does not occur for at least about twenty days during the storage condition.

3. A lysis buffer, comprising:
one or more surfactants;
ammonium sulfate ((NH₄)₂SO₄); and
magnesium sulfate (MgSO₄),
wherein formation of a precipitate in the lysis buffer is substantially inhibited for a period of time under a storage condition, thereby no detectable precipitate is formed in the lysis buffer for at least about twenty days during the storage condition.

4. The lysis buffer of claim 3, wherein the lysis buffer does not comprise one or more of sodium dodecyl sulfate (SDS) and cetyl trimethylammonium bromide (CTAB).

5. The lysis buffer of any one of claims 1-4, wherein the precipitation of complexes consisting of Mg⁺² and the surfactant is suppressed, optionally the concentration of soluble Mg⁺² is not reduced more than about 1.1-fold relative to the start of the period of time.

6. The lysis buffer of any one of claims 1-5, wherein the appearance of the precipitate in the lysis buffer does not occur for at least about thirty days, about sixty days, about ninety days, about six months, about a year, or about 18 months, during the storage condition.

7. The lysis buffer of any one of claims 1-6, wherein the storage condition comprises transport of the lysis buffer.

8. The lysis buffer of any one of claims 1-7, wherein the storage condition comprises thermal stress, one or more freeze-thaw cycles, agitation, pressure changes, light irradiation, or any combination thereof.

9. The lysis buffer of any one of claims 1-8, wherein the storage condition comprises ambient conditions, optionally in the range from about 20° C. to about 25° C.

10. The lysis buffer of any one of claims 1-9, wherein the storage condition comprises refrigeration conditions, optionally a temperature of about 4° C.

11. The lysis buffer of any one of claims 1-10, wherein the storage condition comprises a temperature of 14° C.

12. The lysis buffer of any one of claims 1-11, wherein the period of time is at least about thirty days, about sixty days, about ninety days, about six months, about a year, or about 18 months.

13. The lysis buffer of any one of claims 1-12, wherein the substantial inhibition of formation of the precipitate comprises the lysis buffer having no visible particulates as assessed by visual inspection.

14. The lysis buffer of any one of claims 1-13, wherein the absence of the precipitate in the lysis buffer comprises the lysis buffer as assessed by visual inspection.

15. The lysis buffer of any one of claims 1-14, wherein the lysis buffer further comprises one or more alcohols.

16. The lysis buffer of claim 15, wherein the one or more alcohols have a carbon chain length in the range of 1 to 6, optionally the one or more alcohols are selected from the group consisting of ethanol, isopropanol, isobutyl alcohol, pentanol, and hexanol.

17. The lysis buffer of any one of claims 15-16, wherein the lysis buffer comprises the one or more alcohols at about 0.001% (v/v) to about 5.0% (v/v), optionally about 0.1% (v/v) to about 4.0% (v/v).

18. The lysis buffer of any one of claims 1-17, wherein the MgSO₄ is present at a concentration of about 0.1 mM to about 10 mM, optionally 4 mM.

19. The lysis buffer of any one of claims 1-18, wherein the (NH₄)₂SO₄ is present at a concentration of about 0.1 mM to about 20 mM, optionally the (NH₄)₂SO₄ is present at a concentration of about 5 mM.

20. The lysis buffer of any one of claims 1-19, wherein the (NH₄)₂SO₄ is present at a concentration of about 10 mM, and wherein the appearance of the precipitate in the lysis buffer is delayed by at least about ten days as compared a comparable lysis buffer wherein the (NH₄)₂SO₄ is present at a concentration of about 5 mM.

21. The lysis buffer of any one of claims 1-20, further comprising an acid at a concentration of about 0.1 mM to about 20 mM, optionally the acid comprises an organic acid, an inorganic acid, or a mixture thereof, further optionally the inorganic acid is hydrogen chloride (HCl), optionally the acid is present at a concentration of about 8.8 mM.

22. The lysis buffer of any one of claims 1-21, further comprising a pH buffer.

23. The lysis buffer of claim 22, wherein the pH buffer comprises glycine and an acid, optionally 10.0 mM glycine and HCl, optionally 8.8 mM.

24. The lysis buffer of any one of claims 22-23, wherein the pH of the lysis buffer is about 1.0 to about 4.0, optionally the pH of the lysis buffer is about 2.2.

25. The lysis buffer of any one of claims 1-24, wherein the one or more surfactants are capable of lysing biological entities to release sample nucleic acids comprised therein.

26. The lysis buffer of claim 25, wherein the sample nucleic acids comprise sample ribonucleic acids and/or sample deoxyribonucleic acids.

27. The lysis buffer of any one of claims 25-26, wherein the biological entities comprise a virus, a bacteria, a fungi, a protozoa, portions thereof, or any combination thereof.

28. The lysis buffer of any one of claims 25-27, wherein the biological entities comprise one or more of prokaryotic cells, eukaryotic cells, viral particles, exosomes, protoplasts, and microvesicles.

29. The lysis buffer of any one of claims 1-28, wherein the one or more surfactants comprise about 0.001% (w/v) to about 2.0% (w/v) of the lysis buffer.

30. The lysis buffer of any one of claims 1-29, wherein the one or more surfactants comprise a cationic surfactant, an anionic surfactant, a non-ionic surfactant, or an amphoteric surfactant.

31. The lysis buffer of any one of claims 1-30, wherein the lysis buffer further comprises a non-ionic surfactant, optionally the non-ionic surfactant is selected from the group consisting of Tween 20, Tween 40, Tween 45, Tween 60, Tween 65, Tween 80, Tween 81 and Tween 85, further optionally the non-ionic surfactant comprises about 0.01% (w/v) of the lysis buffer.

32. The lysis buffer of any one of claims 1-31, wherein the one or more surfactants comprises cetyl trimethylammonium bromide (CTAB), optionally the lysis buffer comprises about 0.2% (w/v) CTAB and the (NH₄)₂SO₄ is present at a concentration of about 5 mM.

33. The lysis buffer of any one of claims 1-31, wherein the one or more surfactants comprises cetyl trimethylammonium chloride (CTAC), optionally the lysis buffer comprises about 0.2% (w/v) CTAC and the (NH₄)₂SO₄ is present at a concentration of about 5 mM.

34. The lysis buffer of any one of claims 1-31, wherein the one or more surfactants comprises SDS, optionally the lysis buffer comprises about 0.4% (w/v) SDS and the (NH₄)₂SO₄ is present at a concentration of about 10 mM, optionally the lysis buffer further comprises Tween 80.

35. The lysis buffer of any one of claims 1-31, wherein the one or more surfactants comprises sodium decyl sulfate (SDeS), optionally the lysis buffer comprises about 0.2% (w/v) SDeS and the (NH₄)₂SO₄ is present at a concentration of about 5 mM.

36. The lysis buffer of any one of claims 1-31, wherein the one or more surfactants comprises sodium decyl sulfate (SDeS), optionally the lysis buffer comprises about 0.2% (w/v) SDeS and the (NH₄)₂SO₄ is present at a concentration of about 10 mM.

37. The lysis buffer of any one of claims 1-31, wherein the one or more surfactants comprises SDeS, optionally the lysis buffer comprises about 0.4% (w/v) SDeS and the (NH₄)₂SO₄ is present at a concentration of about 10 mM.

38. The lysis buffer of any one of claims 1-31, wherein the one or more surfactants comprises SDeS, optionally the lysis buffer comprises about 0.8% (w/v) SDeS and the (NH₄)₂SO₄ is present at a concentration of about 10 mM.

39. The lysis buffer of any one of claims 1-31, wherein the one or more surfactants comprises SDeS, optionally the lysis buffer comprises about 0.4% (w/v) SDeS and the (NH₄)₂SO₄ is present at a concentration of about 10 mM, optionally the lysis buffer further comprises Tween 80.

40. The lysis buffer of any one of claims 1-31, wherein the one or more surfactants comprises CTAB, optionally the lysis buffer comprises about 0.2% (w/v) CTAB and the (NH₄)₂SO₄ is present at a concentration of about 5 mM.

41. The lysis buffer of any one of claims 1-31, wherein the one or more surfactants comprises cetyl trimethylammonium chloride (CTAC), optionally the lysis buffer comprises about 0.2% (w/v) CTAC and the (NH₄)₂SO₄ is present at a concentration of about 5 mM.

42. The lysis buffer of any one of claims 1-31, wherein the one or more surfactants comprises sodium octyl sulfate (S Octyl S), optionally the lysis buffer comprises about 0.2% (w/v) S Octyl S and the (NH₄)₂SO₄ is present at a concentration of about 5 mM.

43. The lysis buffer of any one of the claims 1-42, wherein the lysis buffer further comprises a reducing agent, further optionally the reducing agent is present at a concentration of about 0.1 mM to about 100 mM, further optionally the reducing agent is or comprises cysteine.

44. A method of processing a sample, comprising:
(a) contacting a sample comprising biological entities with a lysis buffer of any one of claims 1-43 to generate a treated sample, thereby lysing the biological entities to release sample nucleic acids comprised therein.

45. The method of claim 44, wherein the sample nucleic acids are suspected of comprising a target nucleic acid sequence, and the method further comprising detecting the target nucleic acid sequence in the sample.

46. The method of claim 45, wherein detecting the target nucleic acid sequence in the sample comprises:
(b) contacting a reagent composition with the treated sample to generate an amplification reaction mixture, wherein the reagent composition comprises one or more amplification reagents;
(c) amplifying a target nucleic acid sequence in the amplification reaction mixture, thereby generating a nucleic acid amplification product; and
(d) detecting the nucleic acid amplification product, wherein the detecting is performed in less than about 20 minutes from the time the reagent composition is contacted with the treated sample.

47. The method of any one of claims 44-46, wherein the sample nucleic acids comprise sample ribonucleic acids and/or sample deoxyribonucleic acids, optionally the sample ribonucleic acids comprise a cellular RNA, a mRNA, a microRNA, a bacterial RNA, a viral RNA, or any combination thereof.

48. The method of any one of claims 46-47, wherein the one or more amplification reagents comprise a reverse transcriptase and/or an enzyme having a hyperthermophile polymerase activity, optionally the enzyme having a hyperthermophile polymerase activity has a reverse transcriptase activity.

49. The method of any one of claims 46-48, wherein contacting the reagent composition with the treated sample comprises dissolving the reagent composition in the treated sample.

50. The method of any one of claims 46-49, wherein the reagent composition comprises one or more of a reverse transcriptase, an enzyme having a hyperthermophile polymerase activity, a first primer, a second primer, and a reverse transcription primer.

51. The method of any one of claims 46-50, wherein the amplifying is performed in an isothermal amplification condition.

52. The method of any one of claims 46-51, wherein detecting the nucleic acid amplification product comprises use of a real-time detection method.

53. The method of any one of claims 46-52, wherein the reagent composition is lyophilized and/or heat-dried and comprises one or more additives, wherein the one or more additives comprise:
an amino acid;
a sugar or sugar alcohol, optionally the sugar or sugar alcohol comprises sucrose, lactose, trehalose, dextran, erythritol, arabitol, xylitol, sorbitol, mannitol, or any combination thereof; and/or
a polymer, optionally the polymer comprises polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof.

54. The method of any one of claims 44-53, wherein the sample nucleic acids comprise a nucleic acid comprising the target nucleic acid sequence.

55. The method of any one of claims 45-54, wherein the target nucleic acid sequence comprises a first strand and a second strand complementary to each other.

56. The method of any one of claims 46-55, wherein amplifying the target nucleic acid sequence comprises:
amplifying a target nucleic acid sequence comprising a first strand and a second strand complementary to each other in an isothermal amplification condition, wherein the amplifying comprises contacting a nucleic acid comprising the target nucleic acid sequence with:
i) a first primer and a second primer, wherein the first primer is capable of hybridizing to a sequence of the first strand of the target nucleic acid sequence, and the second primer is capable of hybridizing to a sequence of the second strand of the target nucleic acid sequence; and
ii) an enzyme having a hyperthermophile polymerase activity, thereby generating a nucleic acid amplification product, wherein the nucleic acid amplification product comprises:
(1) the sequence of the first primer, and the reverse complement thereof,
(2) the sequence of the second primer, and the reverse complement thereof, and
(3) a spacer sequence flanked by (1) the sequence of the first primer and the reverse complement thereof and (2) the sequence of the second primer and the reverse complement thereof, wherein the spacer sequence is 1 to 10 bases long.

57. The method of any one of claims 46-56, wherein the amplifying does not comprise using any enzyme other than the enzyme having a hyperthermophile polymerase activity, optionally the amplifying does not comprise heat denaturing and/or enzymatic denaturing the nucleic acid, further optionally the method does not comprise contacting the nucleic acid with a single-stranded DNA binding protein prior to or during step (c).

58. The method of any one of claims 54-57, wherein the nucleic acid is a double-stranded DNA.

59. The method of any one of claims 54-58, wherein the nucleic acid is a product of reverse transcription reaction, optionally the nucleic acid is a product of reverse transcription reaction generated from sample ribonucleic acids, further optionally step (c) comprises generating the nucleic acid by a reverse transcription reaction.

60. The method of any one of claims 44-59, wherein the sample nucleic acids comprise sample ribonucleic acids, and wherein the method comprises contacting sample ribonucleic acids with a reverse transcriptase and/or a reverse transcription primer to generate a cDNA.

61. The method of any one of claims 46-60, wherein amplifying the target nucleic acid sequence comprises:
(c1) contacting sample ribonucleic acids with a reverse transcriptase and/or a reverse transcription primer to generate a cDNA;
(c2) contacting the cDNA with an enzyme having a hyperthermophile polymerase activity to generate a double-stranded DNA (dsDNA), wherein the dsDNA comprises a target nucleic acid sequence, and wherein the target nucleic acid sequence comprises a first strand and a second strand complementary to each other;
(c3) amplifying the target nucleic acid sequence under an isothermal amplification condition, wherein the amplifying comprises contacting the dsDNA with:
(i) a first primer and a second primer, wherein the first primer is capable of hybridizing to a sequence of the first strand of the target nucleic acid sequence, and the second primer is capable of hybridizing to a sequence of the second strand of the target nucleic acid sequence; and
(ii) the enzyme having a hyperthermophile polymerase activity, thereby generating a nucleic acid amplification product, wherein the nucleic acid amplification product comprises:
(1) the sequence of the first primer, and the reverse complement thereof,
(2) the sequence of the second primer, and the reverse complement thereof, and
(3) a spacer sequence flanked by (1) the sequence of the first primer and the reverse complement thereof and (2) the sequence of the second primer and the reverse complement thereof, wherein the spacer sequence is 1 to 10 bases long.

62. The method of claim 61, wherein the method does not comprise using any enzymes other than the reverse transcriptase and the enzyme having a hyperthermophile polymerase activity.

63. The method of any one of claims 46-62, wherein step (d) further comprises determining the amount of the dsDNA and/or nucleic acid that comprises the target nucleic acid sequence in the sample.

64. The method of any one of claims 46-63, wherein the enzyme having a hyperthermophile polymerase activity has an amino acid sequence that is at least about 90% identical to the amino acid sequence of SEQ ID NO: 1 or a functional fragment thereof, optionally the enzyme having a hyperthermophile polymerase activity has an amino acid sequence that is at least about 95% identical to the amino acid sequence of SEQ ID NO: 1, further optionally the enzyme having a hyperthermophile polymerase activity is a polymerase comprising the amino acid sequence of SEQ ID NO: 1.

65. The method of any one of claims 46-64, wherein the enzyme having a hyperthermophile polymerase activity has low or no exonuclease activity.

66. The method of any one of claims 46-65, wherein amplifying the target nucleic acid sequence is performed at a constant temperature of about 55° C. to about 75° C., optionally amplifying the target nucleic acid sequence is performed at a constant temperature of about 65° C.

67. The method of any one of claims 50-66, wherein the first primer, the second primer, and/or the reverse transcription primer is about 8 to 16 bases long, optionally the first primer, the second primer, and/or the reverse transcription primer comprises one or more of DNA bases, modified DNA bases, or a combination thereof.

68. The method of any one of claims 46-67, wherein the nucleic acid amplification product is about 20 to 40 bases long.

69. The method of any one of claims 56-68, wherein the spacer sequence comprises a portion of the target nucleic acid sequence, optionally the spacer sequence is 1 to 10 bases long.

70. The method of any one of claims 46-69, further comprising contacting the nucleic acid amplification product with a signal-generating oligonucleotide capable of hybridizing to the amplification product, wherein the single-generating oligonucleotide comprises a fluorophore, a quencher, or both.

71. The method of any one of claims 46-70, wherein detecting the nucleic acid amplification product comprises detecting a fluorescent signal, optionally the fluorescent signal is from a molecular beacon.

72. The method of any one of claims 44-71, wherein the method is performed in a single reaction vessel.

73. The method of any one of claims 44-72, wherein the biological entities comprise one or more of prokaryotic cells, eukaryotic cells, viral particles, exosomes, protoplasts, and microvesicles.

74. The method of any one of claims 44-73, wherein the biological entities comprise a virus, a bacteria, a fungi, a protozoa, portions thereof, or any combination thereof.

75. The method of any one of claims 45-74, wherein the target nucleic acid sequence is a nucleic acid sequence of a virus, bacteria, fungi, or protozoa, optionally the sample nucleic acids are derived from a virus, bacteria, fungi, or protozoa.

76. The method of any one of claims 74-75,
wherein the virus is SARS-COV-2, Human Immunodeficiency Virus Type 1 (HIV-1), Human T-Cell Lymphotrophic Virus Type 1 (HTLV-1), Hepatitis B Virus (HBV), Hepatitis C Virus (HCV), Herpes Simplex, Herpesvirus 6, Herpesvirus 7, Epstein-Barr Virus, Respiratory Syncytial Virus (RSV), Cytomegalo-virus, Varicella-Zoster Virus, JC Virus, Parvovirus B19, Influenza A, Influenza B, Influenza C, Rotavirus, Human Adenovirus, Rubella Virus, Human Enteroviruses, Genital Human Papillomavirus (HPV), or Hantavirus;
wherein the bacteria is Mycobacteria tuberculosis, Rickettsia rickettsii, Ehrlichia chaffeensis, Borrelia burgdorferi, Yersinia pestis, Treponema pallidum, Chlamydia trachomatis, Chlamydia pneumoniae, Mycoplasma pneumoniae, Mycoplasma sp., Legionella pneumophila, Legionella dumoffii, Mycoplasma fermentans, Ehrlichia sp., Haemophilus influenzae, Neisseria meningitidis, Neisseria gonorrhoeae, Streptococcus pneumonia, S. agalactiae, or Listeria monocytogenes;
wherein the fungi is Cryptococcus neoformans, Pneumocystis carinii, Histoplasma capsulatum, Blastomyces dermatitidis, Coccidioides immitis, or Trichophyton rubrum; and/or
wherein the protozoa is Trypanosoma cruzi, Leishmania sp., Plasmodium, Entamoeba histolytica, Babesia microti, Giardia lamblia, Cyclospora sp., or Eimeria sp.

77. The method of any one of claims 46-76, wherein the amplifying step comprises multiplex amplification of two or more target nucleic acid sequences, and wherein the detecting step comprises multiplex detection of two or more nucleic acid amplification products derived from said two or more target nucleic acid sequences.

78. The method of claim 77, wherein the two or more target nucleic acid sequences are specific to two or more different organisms, optionally the two or more different organisms comprise one or more of SARS-COV-2, Influenza A, Influenza B, and Influenza C.

79. The method of any one of claims 46-78, wherein the amplifying comprises one or more of the following: Archaeal Polymerase Amplification (APA), loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA).

80. The method of any one of claims 46-78, wherein the amplifying does not comprise one or more of the following: APA, LAMP, HDA, RPA, SDA, NASBA, TMA, NEAR, RCA, MDA, RAM, cHDA, SPIA, SMART, 3SR, GEAR and IMDA, and optionally the amplifying does not comprise LAMP.

81. The method of any one of claims 43-80, wherein the method does not comprise one or more of the following: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of ribonuclease inhibitors to the treated sample; (vii) the addition of ribonuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heating denaturing and/or enzymatic denaturing of the sample nucleic acids prior to and/or during amplification; and (xiii) the addition of ribonuclease H to the treated sample or amplification reaction mixture.
